(19) United States
(12) Patent Application Publication
(10) Pub. No.: US 2023/0065158 A1
(43) Pub. Date: Mar. 2, 2023

(54) OXABICYCLOHEPTANES FOR TREATMENT OF SMALL CELL LUNG CANCER

(71) Applicant: Lixte Biotechnology, Inc., Pasadena, CA (US)

(72) Inventors: John S. KOVACH, Pasadena, CA (US); Ravi SALGIA, Duarte, CA (US)

(21) Appl. No.: 17/893,698

(22) Filed: Aug. 23, 2022

Related U.S. Application Data
(63) Continuation of application No. PCT/US2021/051647, filed on Sep. 23, 2021.
(60) Provisional application No. 63/139,047, filed on Jan. 19, 2021.

(51) Int. Cl. A61K 31/4995; A61K 39/395; A61K 31/7048; A61K 31/282; A61P 35/00; A61P 11/00

(52) U.S. Cl. CPC A61K 31/4995; A61K 39/3955; A61K 31/7048; A61K 31/282; A61P 35/00; A61P 11/00

(57) ABSTRACT

The present invention provides a method of treating a subject suffering from SCLC comprising administering to the subject an effective amount of a PP2A inhibitor and optionally one or more anti-cancer agents.

### CROSS-REFERENCE TO RELATED APPLICATIONS

The present application claims the benefit of U.S. Provisional Patent Application No. _{63/139,047}, filed Jan. 19, 2021, the entirety of which is incorporated herein by reference thereto.

### TECHNICAL FIELD OF THE INVENTION

The present invention relates to methods useful for inhibiting phosphatase 2A (PP2A) in a subject in need thereof.

### BACKGROUND OF THE INVENTION

Protein phosphatase 2A (PP2A) is a ubiquitous serine/threonine phosphatase that dephosphorylates numerous proteins of both ATM/ATR-dependent and -independent response pathways (Mumby, M. 2007). Pharmacologic inhibition of PP2A has previously been shown to sensitize cancer cells to radiation-mediated DNA damage via constitutive phosphorylation of various signaling proteins, such as p53, γH2AX, PLK1 and Akt, resulting in cell cycle deregulation, inhibition of DNA repair, and apoptosis (Wei, D. et al. 2013).

Cantharidin, the principle active ingredient of blister beetle extract (Mylabris), is a compound derived from traditional Chinese medicine that has been shown to be a potent inhibitor of PP2A (Efferth, T. et al. 2005). Although cantharidin has previously been used in the treatment of hepatomas and has shown efficacy against multidrug-resistant leukemia cell lines (Efferth, T. et al. 2002), its severe toxicity limits its clinical usefulness. LB-100 (i.e., (3-[(4-Methylpiperazin-1-yl)carbonyl]-7-oxabicyclo[2.2.1]heptane-2-carboxylic acid]), is a small molecule derivative of cantharidin with significantly less toxicity. Previous pre-clinical studies have shown that LB-100 can enhance the cytotoxic effects of temozolomide, doxorubicin, and radiation therapy against glioblastoma (GBM), metastatic pheochromocytoma, and pancreatic cancer (Wei, D. et al. 2013; Lu, J. et al. 2009; Zhang, C. et al. 2010; Martiniova, L. et al. 2011). LB-100 is also undergoing a phase 1 study in combination with docetaxel for the treatment of solid tumors (Chung, V. 2013).

More than one million people died from lung cancer worldwide in 2017, and small cell carcinomas account for approximately 15% of all lung cancers. Even with double or triple drug therapy combinations, median survival for small cell lung carcinoma (SCLC) with “extensive disease” (ED-SCLC, 70% of patients) is only approximately 9 months and overall 5-year survival remains at around 5%. PP2A is ubiquitously expressed in SCLC cells, however, its potential relevance in SCLC remains mostly unknown. Protein phosphatase 2A (PP2A) is a phosphatase involved in the regulation of key oncoproteins, such as c-Myc and Bcr-Abl in a wide range of cancer subtypes including lung cancers and B cell-derived leukemias. Accordingly, there remains a need for improved treatments for patients suffering from SCLC, and in particular, ED-SCLC. The present invention encompasses the recognition that LB-100, either alone or in combination with one or more anti-cancer agents, is useful in treating patients suffering from SCLC, for instance, ED-SCLC.

### SUMMARY OF THE INVENTION

The present invention provides, inter alia, methods of treating a subject suffering from small cell lung carcinoma (SCLC) comprising administering to the subject an effective amount a compound of the following structure, referred to herein as LB-100 (i.e., (3-[(4-Methylpiperazin-1-yl)carbonyl]-7-oxabicyclo[2.2.1]heptane-2-carboxylic acid]):

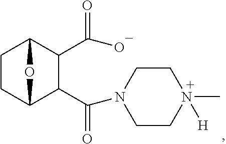

or a pharmaceutically acceptable salt, zwitterion, or ester thereof.

In some embodiments, the present invention provides a method of treating a subject suffering from SCLC comprising administering LB-100 in combination with one or more anti-cancer agents, wherein the amounts when taken together are effective to treat the subject.

In some embodiments, the present invention provides a method of treating a subject suffering from SCLC and receiving one or more anti-cancer agents comprising administering to the subject of an amount of LB-100 effective to enhance treatment relative to the one or more anti-cancer agent administered in the absence of LB-100.

In some embodiments, the one or more additional anti-cancer agents are selected from carboplatin, etoposide, and atezolizumab. In some embodiments, the one or more additional anti-cancer agents are carboplatin, etoposide, and atezolizumab.

In some embodiments, the SCLC is untreated extensive stage SCLC (ED-SCLC).

### BRIEF DESCRIPTION OF THE DRAWINGS

FIGS. 1A-IL depict the effects of LB-100 on PP2A-A expression in SCLC tumors and cells. FIG. 1A: Scatter plot shows an upregulation of the PP2A-A subunit in the tumor samples. A Mann-Whitney U test was used for comparison between the normal and SCLC samples. FIG. 1B: IHC for PP2A was conducted on TMA tissue sections and images were captured at 4× or 20× using a 3D-Histech PANNORAMIC SCAN whole slide scanner (3D-Histech, Budapest, Hungary). PP2A subunit A positively immunostained the cytoplasm and nucleus of normal lung and tumor tissue, but was highly upregulated in tumor tissue. TMAs were scored in normal (n=24) and tumor (n=79) cores on a scale from 0 (no staining/no protein expression) to 3+(strong staining/high protein expression). FIG. 1C: Summary bar graph of the average PP2A subunit staining. IHC staining intensity of normal and tumor cores. There was a statistically significant difference between normal and tumor tissue (p<0.001). FIG. 1D: In order to compare the expression of PP2A subunits A and C, cell lysates from seven SCLC cell lines and HBEC 3KT (non-malignant cell line) were subjected to western blotting. FIG. 1E: PP2A activity was determined using a serine/threonine phosphatase activity assay (Millipore) after 24 h exposure to cantharidin (10 μM) and LB-100 (5 μM). FIG. 1F: The inset showed reduction of PP2A subunit Aα in H524 cells as well as inhibition of cell proliferation due to PP2A subunit Aα knockdown(p<0.5) (n=3). LB-100 alone or in combination with carboplatin inhibited proliferation and colony formation in SCLC cells. The Cell Counting Kit-8 assay detected cell H524 and H69 cell viability. FIGS. 1G and 1H: Cells were treated with LB-100, carboplatin and etoposide, as a single treatment or in combination, at constant ratio. The combination index (CI) was calculated using Chou-Talalay method to find synergism between LB-100 with carboplatin and etoposide (CompuSyn software: www.combosyn.com). FIGS. 1I-1K: Graphs depict the mean+SEM of percent viability for cells (n=3). Colony formation assays were used to count the ability of H524 (FIGS. 11 and 1J) and H69 (FIGS. 1K and 1L) cells to form colonies. Drug concentrations are listed for two assays with H524 and H69 respectively: LB-100 (2.5 μM; 20 μM), carboplatin (4 μM; 20 μM), etoposide (3 μM; 30 μM), LB-100/carboplatin (2.5&4 μM; 20&20 μM) and LB-100/etoposide (2.5&3 μM; 20&30 μM). Representative images of colonies at 4× are shown under the graph (n=2).*p<0.05; **, p<0.01; *** p<0.001; ****, p<0.0001. Experiments were repeated in triplicate and representative data are shown.

FIGS. 2A-2H depict the effect of LB-100 on H446 spheroid growth. FIG. 2A: Morphology of a single spheroid of H446 cells on days one and nine. Spheroids grow continuously and H&E staining is represented. FIG. 2B: Spheroid's growth in response to LB-100 treatment was recorded with IncuCyte Live-Cell Analysis System. FIG. 2C: Cytotoxicity effect of LB100 was recorded with IncuCyte Live-Cell Analysis System in the presence of LB-100 and IncuCyte Cytotox reagent in green fluorescence. Effect of LB-100, carboplatin, etoposide, and drug combination on H446 spheroid morphology and growth (FIG. 2D) Representative images of H&E-stained H446 spheroids with LB-100, carboplatin, etoposide, and combination treatment. Scale bar 100 μm. (FIGS. 2E and 2G). Effect of LB100 and carboplatin alone or in combination was monitored using IncuCyte Live Cell system for 70 h. Maximal significant inhibitory effect of LB-100, carboplatin or drug combination on spheroid's size was observed at time point 70 hours. FIGS. 2F and 2H: Effect of LB-100 and etoposide alone or in combination was monitored using IncuCyte Live Cell system for 72 h. Maximal significant inhibitory effect of LB-100, carboplatin or drug combination on spheroid's size was observed at time point 70 and 72 h (n=3). *, p<0.05; **,p<0.01.

FIGS. 3A-3H depict SCLC cell invasion through HUVEC monolayer. FIGS. 3A-3D: Graphical representation of H524/H69 cell ability to disrupt a confluent HUVEC monolayer using an electrical substrate-impedance sensing system. Arrows indicate time point when cells were added. Inserts show mean values and SD for each group after 20 h of drug treatment. After treatment, cell viability was counted using an Auto T4 Cell Counter (Nexcelom Cellometer). Cell viability was 90-95% for drug-treated groups (n=2). p<0.001 (***) for control (untreated cells) vs. drug combination (LB100/carboplatin). Whole cell Pt accumulation. Graphical representation of LB-100 effect on platinum uptake by SCLC cells. Cells were pretreated with LB-100 (H524—5 μM; H69—20 μM) overnight, then treated with carboplatin for one or four hours (H524—10 μM; H69—30 μM). Whole cell pellet was used for platinum (Pt) measurement. Values are normalized to total protein concentration. FIGS. 3E and 3F: Panels show mean values and SD of Pt accumulation for each group. Drug combination significantly increased Pt concentrations in H524 and H69 cells. Pt concentrations in control and LB-100 samples were below detection limit (n=3, technical replicates). Effect of LB-100 on PP2A expression and apoptosis regulatory proteins in H524 and H69 cells. Cells were treated with indicated concentrations of LB-100, carboplatin and combination for 72 h. FIG. 3G: Representative western blot (WB) panels of the expression of PP2A subunits in H524 and H69 cells (n=3).

FIG. 3H: Protein phosphorylation of γ-H2AX, caspase 3 and PARP1 cleavage activity was analyzed by WB in H524 and H69 cells after drug treatments (n=3). Representative WB panels showed significant increase in γ-H2AX phosphorylation and enhancement of caspase 3 and PARP 1 cleavage activity in H524 and H69 cells after treatment. Pan-actin was used as loading control (n=3).

FIGS. 4A-4F depict a reactome pathway analysis of PamGene PTKs and STKs after LB-100 treatment of H524 cells and Biolog phenotype MicroArray. FIG. 4A: Significant changes were observed for signal transduction and metabolic pathways. FIG. 4B: MicroArray analysis showed that overnight treatment with 20 μM treatment with LB100 inhibited utilization of carbon substrate sources. Table includes 10 carbon sources affected by LB-100 (n=3). FIG. 4C: LB-100 significantly inhibited two carbon substrates utilization by H69 cells. P<0.001 (***) for control (untreated cells) vs. LB-100. FIG. 4D: Amplex Red Glucose/Oxidase assay kit was used to measure glucose level in cell culture media. Glucose level was significantly higher in cell culture medium from cells treated with LB-100 (20 μM). Glucose concentration detected in initial medium and counted as 100%. Subtracting final medium level of glucose from initial glucose medium concentration yielded % glucose in the medium with cells (n=3). Effect of LB-100 on MET phosphorylation. FIG. 4E: H524 and H69 cells were treated overnight with LB-100 (H524—5 μM and H69—20 μM) following by stimulation with 100 ng/ml HGF in 10 min. Cells were collected and lysed for WB analysis with pMET and total MET antibody. Pan-actin was used as loading control (n=3). FIG. 4F: H524 cell lysates (control, LB-100, carboplatin and combination (LB-100/carboplatin) were analyzed by western blots to check phosphorylation status of MET at Ser985 and Tyr1234/1235. Actin was used as a loading control (n=3).

FIGS. 5A-5F depict the effect of LB-100 on cell energy phenotype in SCLC cells. FIG. 5A: LB100 treatment (2.5 μM) induced metabolic switch in H524 cells. Cell energy phenotype was obtained by using XF Cell Energy Phenotype Reporter Generator. Empty squares indicate baseline energy phenotype, solid squares represent stressed energy phenotype measured after oligomycin/FCCP injection. FIGS. 5B and 5C: OCR and ECARin control (blue circles) and LB-100 (orange circles) H524 cells were measured overtime. (n=2, six different wells for each study participant). FIG. 5D: Effect of LB-100 (10 μM) on H69 cell energy phenotype. FIGS. 5E and 5F: Effect of mitochondrial stressor on OCR and ECAR in H69 cells. Blue circles indicate control and orange circles show LB-100 treatment. (n=2, six technical replicates).

FIGS. 6A-6J depict ATP production rate in SCLC cells. FIG. 6A: H524 cells were treated with LB100 (2.5 μM), carboplatin (4 μM), or a combination, and ATP production rate was measured using the Agilent Seahorse XF Real Time ATP rate assay. mitoATP (mitochondrial) and glycoATP (glycolityc) rates were evaluated in H524 cells without and with drug treatments. All drug treatments significantly reduced mitoATP (top, blue) and glycoATP (bottom, red) production rates. FIG. 6B: Energetic map of H524 cells. After LB-100 and drug combination, cells became less glycolytic. FIGS. 6C-6E: The Agilent Seahorse XF pH sensor probe measures changes in the concentration of free protons, which corresponds to Extracellular Acidification Rate (ECAR). Real Time ATP rate assay includes an improved metric, Proton Efflux Rate (PER), which detects extracellular acidification from all sources. LB-100 drastically reduced PER under basal conditions and after two injections of specific inhibitors of oxidative phosphorylation oligomycin (1.5 μM) and antimycin (0.5 μM)/rotenone (0.5 μM). FIG. 6F: H69 cells were treated with LB-100 (10 μM), carboplatin (10 μM), or a combination with LB100/carboplatin. ATP level in cells was measured using the Agilent Seahorse XF Real Time ATP rate assay. LB-100, carboplatin and combination significantly reduced mitoATP. FIG. 6G; Energetic map of H69 cells. FIGS. 6H-6J: H69 cellular Proton Efflux Rate after LB100 treatment from glycolysis of basal and olygomycin and antimycin/rotenone injections. (n=2, six technical replicates).

FIGS. 7A-7G depict results of T cells infiltration in H446 spheroids in the presence of LB100 and atezolizumab. FIG. 7A: Schematic of the effect of activated T cells on H446 spheroid degradation. At time point 0, single spheroids in 96 well plate were treated with LB-100, atezolizumab and T cells. Beads mimic in vivo T cell activation by two action signals CD3 and CD28. IncuCyte® Live-Cell Analysis System was used for the spheroidal imaging. Right panel presents spheroidal degeneration after 48 h incubation with LB-100, atezolizumab and activated T cells. FIGS. 7B and 7C: Automated image analysis provides metrics (0 h-μm, 48 h-mm) and spheroid area (yellow-bright field mask). Column bars present mean values of spheroids at 0h. Representative images in bright field mask. FIGS. 7D and 7E: Measurement of H446 spheroidal cell distribution after 48 h LB-100 and atezolizumab treatments in the presence of T cells. Images represent regions covered by H446 cells. FIG. 7F: Sequential images of the same H446 spheroids in control and treated groups. Scale bar 400 μm FIG. 7G: H&E and immunohistochemical staining (IHC) with CD3 antibody of H446 spheroids after 48 h of treatments. Scale bar 50 μm. Before treatment, 5×103 cells were seeded in round bottom 96 well plate and grown for 3 days.

FIGS. 8A-8E depicts results of LB-100 activity alone and with carboplatin against H69 cells mouse xenograft. Tumor size (FIG. 8A) and body weights (FIG. 8B) were measured. Inhibition of tumor growth after LB-100 (*p<0.05), carboplatin (***p<0.001) and their combination (***p<0.001) were delivered via i.p. injections. P values show significant differences compared with vehicle group. FIG. 8C: Tumor images from vehicle and drug-treated groups. FIG. 8D: Tumor mass was measured at the end of experiment. Compared with vehicle group, LB-100 or carboplatin alone, or a combination of LB-100 with carboplatin significantly reduced tumor mass. FIG. 8E: Columns show total platinum (Pt) concentration in mouse tumors with carboplatin and LB-100/carboplatin treatments (n=3 as technical replicates) Pt mass was normalized to tumor total mass. Statistical analysis was performed using an ANOVA with Tukey post hoc-test (*p<0.05), carboplatin (**p<0.01).

FIG. 9 depicts evaluation of certain mouse tumors via H&E staining. H&E staining of mouse tumors (A) showed dense nuclear staining and high number of mitotic cells. Treatment with LB100 or carboplatin increased the necrotic area in tumor tissue and combined treatment contained fewer tumor cells. IHC staining with PP2A A, pMET, CD31 (for angiogenesis) and Ki-67 (for cell proliferation) antibodies indicated reduction of staining intensity in tumor sections with combined treatment. Representative images of tumor sections are shown for each group. Scale bar 100 μm.

FIG. 10 depicts the phase I clinical trial study diagram.

### DETAILED DESCRIPTION OF THE INVENTION

As described in further detail below and herein, in some embodiments, the present invention provides a method of treating a subject suffering from small cell lung carcinoma (SCLC) comprising administering to the subject an effective amount of a PP2A inhibitor of the following structure, referred to herein as “LB-100” (i.e., (3-[(4-Methylpiperazin-1-yl)carbonyl]-7-oxabicyclo[2.2.1]heptane-2-carboxylic acid]):

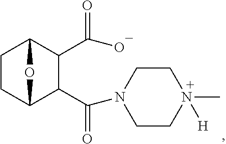

or a pharmaceutically acceptable a salt, zwitterion, or ester thereof. Methods of preparation of LB-100 may be found in at least U.S. Pat. No. 7,998,957 B2 and U.S. Pat. No. 8,426,444 B2.

Protein phosphatase 2A (PP2A) is a ubiquitous serine/threonine phosphatase that is a master tumor suppressor involved in key regulation of oncoproteins, such as c-MYC and BCR-ABL in lung cancer and other cancer types. It has a broad range of cellular regulatory functions such as cell survival, apoptosis, mitosis, and DNA-damage response (13). Previous studies and more recently a Phase I clinical trial have shown that PP2A inhibition can potentially sensitize tumors to radiation and chemotherapy (14). In a Phase I clinical trial of LB-100 in advanced solid tumors LB-100 was well tolerated and 10 out of 20 patients had achieved stable disease (15). Given the ubiquity of PP2A, the inhibition of LB-100 likely has multiple downstream effects. Preclinical studies indicate that PP2A inhibition with LB-100 can result in down regulation of DNA-damage response (16-18) abrogation of cell cycle checkpoint (16, 19), increase HIF dependent tumor angiogenesis (20), and induction of cellular differentiation by inhibition of N-CoR complex formation (16).

Moreover Xiao et al. 2018 showed that PP2A redirected glucose carbon utilization from glycolysis to the pentose phosphate pathway (PPP) to salvage oxidative stress, revealing a gatekeeper function of the PPP in a broad range of B cell malignancies that can be efficiently targeted by small molecule inhibition of PP2A and G6PD(21).

As described above, LB-100 (3-(4methylpiperazine-carbonyl)-7 oxalobicyclo[2.2.1]heptane-2-carboxylic acid; NSC D753810) is a small molecule (MW 268) inhibitor of protein phosphatase 2A (PP2A) and inhibits PP2A about 80 fold more efficiently than protein phosphatase 1 (PP1). The compound has single agent activity in vitro and in vivo. By way of non-limiting theory, the mechanism of potentiation appears to be inhibition of cell cycle and mitotic checkpoints induced by non-specific DNA damaging agents, allowing dormant cancer cells to enter S phase and continue in mitosis despite acute DNA damage (22). Also by way of non-limiting theory, LB-100 appears to affect the vasculature inducing transient reversible vessel “leakiness” at high doses. Because of its unique mechanism of action, LB-100 has the potential to be useful for the treatment of many types of cancer as well as being the first-in-class of a new type of signal transduction modulator.

### Small Cell Lung Carcinoma

Lung cancer is the leading cause of cancer mortality worldwide, with one million new cases annually. Small cell lung cancer (SCLC) is an aggressive form of cancer that is strongly associated with cigarette smoking. In the United States, in 2010, 222,000 new cases of lung cancer were diagnosed, of which 35,000 were SCLC (American Cancer Society). The median age of SCLC patients is 63, and more than 25% are over the age of 70 (1). Small cell lung cancer is a rapidly growing tumor with a high rate of metastases in comparison to non-small cell lung cancer (NSCLC). Patients are staged according to a two-stage system, which was developed by the Veterans Administration Lung Cancer Study Group, consisting of limited-stage disease (LD-SCLC) or extensive-stage disease (ED-SCLC)(2). Limited-stage disease SCLC is confined to a single hemithorax region within an acceptable radiation field. Approximately 65% to 70% of patients with SCLC present with ED-SCLC, which is found beyond a hemithorax region. Untreated patients with ED-SCLC have a median survival of approximately 5 weeks; patients treated with chemotherapy have a median survival of 7 to 11 months (3). ED-SCLC has a 2-year survival rate of less than 10% with current management options.

Combination chemotherapy remains the focus of treatment for patients with ED-SCLC. One of skill in the medical arts will appreciate the challenges associated with such therapies, as in vivo interactions between two or more drugs are often complex. The effects of any single drug are related to its absorption, distribution, metabolism, and elimination. When two drugs are introduced into the body, each drug can affect the absorption, distribution, metabolism, and elimination of the other and hence, alter the effects of the other. For instance, one drug may inhibit, activate or induce the production of enzymes involved in a metabolic route of elimination of the other one or more drugs. (Guidance for Industry, 1999) Thus, when two or more drugs are administered to treat the same condition, it is unpredictable whether such will complement, have no effect on, or interfere with the therapeutic activity of the other in a human subject.

Not only may the interaction between two or more drugs affect the intended therapeutic activity of each drug, but the interaction may increase the levels of toxic metabolites (Guidance for Industry, 1999). The interaction may also heighten or lessen the side effects of each drug. Hence, upon administration of two or more drugs to treat a disease, it is unpredictable what change will occur in the negative side effect profile of each drug.

Additionally, it is difficult to accurately predict when the effects of the interaction between the two or more drugs will become manifest. For example, metabolic interactions between drugs may become apparent upon the initial administration of the second or further drug, after the two have reached a steady-state concentration or upon discontinuation of one of the drugs. (Guidance for Industry, 1999)

In the context of SCLC, in the 1970s and early 1980s, CAV (cyclophosphamide, doxorubicin, and vincristine) was the most commonly used combination regimen. In the mid-1980s, etoposide was discovered as an active agent in SCLC, and preclinical investigations demonstrated synergy between etoposide and cisplatin. Randomized clinical studies confirmed that this combination was as effective as CAV, with less toxicity (3).

Several other agents have been shown to have activity in SCLC, and many studies have compared 3-drug regimens to the standard 2-drug regimens with no improvement in efficacy. A Phase 3 trial conducted by the Norwegian Lung Cancer Study Group randomized 436 patients, including 214 patients with LD-SCLC and 222 patients with ED-SCLC. Patients received etoposide plus cisplatin or a combination of cyclophosphamide, epirubicin, and vincristine (CEV). Median survival for patients with ED-SCLC was 8.4 months in the etoposide plus cisplatin arm and 6.5 months in the CEV arm (p=0.21) (4).

In 2005, Phase 3 study conducted by the Cancer and Leukemia Group B (CALGB) compared the combination etoposide/cisplatin with or without paclitaxel and granulocyte colony-stimulating factor (G-CSF) in patients with ED-SCLC (5). A total of 565 patients were randomized. Median progression-free survival time on the carboplatin/etoposide arm was 5.9 months compared with 6 months for patients receiving carboplatin/etoposide/paclitaxel, and median overall survival was 9.9 months on the etoposide/cisplatin arm and 10.6 months on the paclitaxel arm. Toxic deaths occurred in 2.4% of the patients not receiving paclitaxel and 6.5% of patients being treated with paclitaxel. Thus, the addition of paclitaxel to etoposide and cisplatin did not improve survival and was associated with unacceptable toxicity in patients with ED-SCLC (5).

Results from one of the largest studies ever conducted for patients with ED-SCLC were also reported in 2005. This study included 784 patients randomized to receive either topotecan plus cisplatin or the standard etoposide plus cisplatin; efficacy was comparably seen in overall response rates (63% versus 69%), median time to progression (24.1 versus 25.1 weeks), median survival (39.3 versus 40.3 weeks), and 1-year survival rates (31.4% for both arms) (6).

More recently the phase III IMpower133 randomized double-blind study evaluated whether adding a checkpoint inhibitor of programmed death signaling (atezolizumab) might improve chemotherapy benefits in patients with ED-SCLC (7). A total of 201 patients were randomly assigned to the platinum/etoposide/atezolizumab arm and 202 were assigned to the placebo arm. The median progression-free survival time on the platinum/etoposide arm was 4.3 months as compared with 5.2 months with platinum/etoposide/atezolizumab. The median overall survival was 12.3 months in the platinum/etoposide/atezolizumab arm and 10.3 months in the placebo group. The addition of immunotherapy to etoposide and platinum chemotherapy improved overall survival and progression-free survival and was not associated with unacceptable toxicity in patients with ED-SCLC (7). IMpower133 is considered the first study in 20 years to show a clinically meaningful improvement in overall survival over the standard of care in frontline ED-SCLC.

Carboplatin has been studied in a variety of human solid tumors (ovarian, head and neck, non-small cell lung, and small cell lung) with objective response rates between 10% and 85%. It has also been used successfully in combination with a number of other cytotoxic agents for the treatment of ovarian cancer, NSCLC, and SCLC (8-10). A 1992 review of Phase 2 and 3 studies with carboplatin in patients with SCLC determined carboplatin to be an active agent in untreated SCLC (11).

Platinum-based therapy (carboplatin or cisplatin) combined with etoposide is a current standard of care for patients with ED-SCLC. However, carboplatin is often preferred over cisplatin, as it provides advantages such as fewer gastrointestinal, renal, auditory, and neurologic toxicities as well as easier administration (12).

### Carboplatin Etoposide Atezolizumab as First Line Treatments

Carboplatin is an analog of cisplatin that has a more favorable toxicity profile (Ruckdeschel 1994). It interacts with DNA and forms both intra- and interstrand links. The most commonly observed side effects include thrombocytopenia, neutropenia, leukopenia, and anemia. Like other platinum-containing compounds, carboplatin may induce anaphylactic-type reactions such as facial edema, wheezing, tachycardia, and hypotension that may occur within a few minutes of drug administration. These reactions may be controlled with adrenaline, corticosteroids, or antihistamines (see package insert for further information).

Etoposide is a semisynthetic derivative of podophyllotoxin that exhibits cytostatic activity in vitro by preventing cells from entering mitosis or by destroying them at a premitotic stage. Etoposide interferes with the synthesis of DNA and appears to arrest human lymphoblastic cells in the late S-G2 phase of the cell cycle. The most commonly observed side effects include leukopenia and thrombocytopenia (see package insert for further information).

Etoposide is indicated in combination with other antineoplastics in the treatment of SCLC, NSCLC, malignant lymphoma, and testicular malignancies. Approved indications may vary depending on the specific country. Etoposide is also used in clinical studies against many other types of cancer including head and neck, brain, bladder, cervical, and ovarian.

Atezolizumab is a humanized immunoglobulin (Ig) G1 monoclonal antibody that targets programmed death receptor 1 ligand (PD-L1) and inhibits the interaction between PD-L1 and its receptors, programmed death receptor 1 (PD-1) and B7-1 (also known as CD80), both of which function as inhibitory receptors expressed on T cells. Intravenous atezolizumab has been approved in the US and Europe for the treatment of adult patients with advanced urothelial carcinoma that have failed or are ineligible for a platinum based regimen.(25, 26) Additionally, atezolizumab in combination with bevacizumab, paclitaxel, and carboplatin has been approved in the US for the first-line treatment of adult patients with metastatic NSCLC with no EGFR or ALK genomic tumor aberrations and as monotherapy in locally advanced and metastatic NSCLC after prior chemotherapy.(27) Recently, atezolizumab was also granted accelerated approval in the US, in combination with nab-paclitaxel for patients with unresectable locally advanced or metastatic triple negative breast cancer whose tumors express PD-L1.(28) Finally, atezolizumab was approved for first-line treatment, in combination with carboplatin and etoposide, in adult patients with extensive-stage small cell lung cancer, showing improved survival (median OS 12.3 months in the platinum/etoposide/atezolizumab arm vs. 10.3 months platinum/etoposide/placebo). The addition of immunotherapy to etoposide and platinum chemotherapy in ED-SCLC also improved progression-free survival and was not associated with unacceptable toxicity. (7) Treatment with atezolizumab is generally well-tolerated, but can be associated with immune-related adverse events (irAEs) (see package insert for further information).

### Methods of the Present Invention

As described above and herein, the present invention encompasses the surprising finding that LB-100 is useful in the treatment of subjects suffering from SCLC.

In some embodiments, the present invention provides a method of treating a subject suffering from SCLC comprising administering LB-100 alone or in combination with one or more anti-cancer agents, wherein the amounts when taken together are effective to treat the subject. In some such embodiments, the SCLC is ED-SCLC.

In some embodiments, the present invention provides a method of treating a subject suffering from SCLC and receiving one or more anti-cancer agents comprising administering to the subject of an amount of LB-100 effective to enhance treatment relative to the one or more anti-cancer agent administered in the absence of LB-100. In some such embodiments, the SCLC is ED-SCLC.

In some embodiments, the one or more additional anti-cancer agents are selected from carboplatin, etoposide, and atezolizumab. In some embodiments, the one or more additional anti-cancer agents are each of carboplatin, etoposide, and atezolizumab.

In some embodiments, the SCLC is untreated extensive stage SCLC (ED-SCLC).

In some embodiments, the amount of LB-100 and the amount of the one or more anti-cancer agents are each periodically administered to the subject. Exemplary such methods of administration are described further herein.

In some embodiments, the one or more anti-cancer agents are independently administered concurrently with, prior to, or after administration of LB-100. In some embodiments, the one or more anti-cancer agents are independently administered after administration of LB-100.

In some embodiments, the amount of LB-100 and the amount of the one or more additional anti-cancer agents when taken together are effective to reduce a clinical symptom of the cancer in the subject, as described further herein.

In some embodiments, the amount of LB-100 is effective to reduce a clinical symptom of the cancer in the subject. In some embodiments, LB-100 is administered at a dose of between about 0.25 mg/m2 and about 3.10 mg/m2. In some embodiments, LB-100 is administered at a dose of between about 0.83 mg/m2 and about 3.10 mg/m2. In some embodiments, LB-100 is administered at a dose of between about 0.83 mg/m2 and about 2.33 mg/m2. In some embodiments, LB-100 is administered at a dose of between about 0.83 mg/m2 and about 1.75 mg/m2. In some embodiments, LB-100 is administered at a dose of 0.25 mg/m2, 0.5 mg/m2, 0.83 mg/m2, 1.25 mg/m2, 1.75 mg/m2, 2.33 mg/m2, or 3.10 mg/m2.

In some embodiments, LB-100 is administered at a dose of 0.83 mg/m2.

In some embodiments, LB-100 is administered at a dose of 1.25 mg/m2.

In some embodiments, LB-100 is administered at a dose of 1.75 mg/m2.

In some embodiments, LB-100 is administered at a dose of 2.33 mg/m2.

In some embodiments, LB-100 is administered at a dose of 3.10 mg/m2.

In some embodiments, LB-100 is administered for 1, 2, or 3 days every 3 weeks. In some embodiments, LB-100 is administered on days 1 and 3 of a 21 day cycle. In some such embodiments, LB-100 is administered intravenously. In some such embodiments, LB-100 is administered at a dose of about 0.83 mg/m2. In some such embodiments, LB-100 is administered at a dose of about 1.25 mg/m2. In some such embodiments, LB-100 is administered at a dose of about 1.75 mg/m2. In some such embodiments, LB-100 is administered at a dose of about 2.33 mg/m2. In some such embodiments, LB-100 is administered at a dose of about 3.10 mg/m2.

In some such embodiments, LB-100 is administered at a dose of about 0.83 mg/m2 on days 1 and 3 of a 21 day cycle for at least two cycles. In some such embodiments, LB-100 is administered at a dose of about 0.83 mg/m2 on days 1 and 3 of a 21 day cycle for at least three cycles. In some such embodiments, LB-100 is administered at a dose of about 0.83 mg/m2 on days 1 and 3 of a 21 day cycle for at least four cycles. In some such embodiments, LB-100 is administered at a dose of about 0.83 mg/m2 on days 1 and 3 of a 21 day cycle for at least five cycles. In some such embodiments, LB-100 is administered at a dose of about 0.83 mg/m2 on days 1 and 3 of a 21 day cycle for the life of the patient.

As described further above and herein, in some embodiments the one or more anti-cancer agents comprises carboplatin. In some such embodiments, the carboplatin is administered at a dose corresponding to about AUC 5. In some such embodiments, the carboplatin is administered at a dose that achieves about AUC 5. In some such embodiments, the carboplatin is administered at a dose of up to about 750 mg/day. In some embodiments, the carboplatin is administered in an amount according to the Standard of Care for the subject in need thereof.

In some embodiments, the carboplatin is administered on day 1 of a 21 day cycle. In some embodiments, the carboplatin is administered on day 1 of a 21 day cycle for at least 4 cycles. In some such embodiments, the carboplatin is administered intravenously.

As described further above and herein, in some embodiments the one or more anti-cancer agents comprises atezolizumab. In some such embodiments, the atezolizumab is administered at a dose of about 1200 mg/day. In some embodiments, the atezolizumab is administered in an amount according to the Standard of Care for the subject in need thereof.

In some embodiments, the atezolizumab is administered on day 1 of a 21 day cycle. In some embodiments, the atezolizumab is administered on day 1 of a 21 day cycle for at least 4 cycles. In some such embodiments, the atezolizumab is administered intravenously.

As described further above and herein, in some embodiments the one or more anticancer agents comprises etoposide. In some embodiments, the etoposide is administered at a dose of about 100 mg/m² per day. In some embodiments, the etoposide is administered in an amount according to the Standard of Care for the subject in need thereof.

In some embodiments, the etoposide is administered on days 1, 2, and 3 of a 21 day cycle. In some embodiments, the etoposide is administered on days 1, 2, and 3 of a 21 day cycle for at least 4 cycles. In some embodiments, the etoposide is administered intravenously.

In some embodiments, the present invention provides methods of administering LB-100 in combination with atezolizumab, carboplatin, and etoposide, in any of the amounts and administration regimens described above and herein. In some such embodiments, wherein the one or more anticancer agents comprise each of atezolizumab, carboplatin, and etoposide, the order of administration when administered sequentially in combination on the same day comprises administration of LB-100, followed by administration of atezolizumab, followed by administration of carboplatin, followed by administration of etoposide. In some embodiments, the order of administration is maintained in the absence of administration of one or more of the anticancer agents.

In some embodiments, a subject is treated for at least one, two, three, or four cycles comprising LB-100 and the one or more anti-cancer agents. In some embodiments, a subject is subsequently put on maintenance treatment. For instance, in some embodiments a maintenance treatment comprises LB-100 and atezolizumab administered according to any of the methods described above and herein.

In some embodiments, the subject suffering from SCLC has had no prior systemic chemotherapy, immunotherapy, biological, hormonal, or investigational therapy for SCLC.

In some embodiments, the subject suffering from SCLC has not been diagnosed with NSCLC or mixed NSCLC and SCLC.

In some embodiments, the present invention provides a method wherein the subject is administered a pharmaceutical composition comprising LB-100 and at least one pharmaceutically acceptable carrier for treating the cancer in the subject.

In some embodiments of any of the above methods or uses, the subject is a human.

In some embodiments of any of the above methods or uses, LB-100 and/or the one or more additional anti-cancer agents is orally or parenterally administered to the subject.

As used herein, “treatment of the diseases” or “treating” encompasses inducing prevention, inhibition, regression, or stasis of the disease or a symptom or condition associated with the disease.

As used herein, “inhibition” of disease progression or disease complication in a subject means preventing or reducing the disease progression and/or disease complication in the subject.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

The following delivery systems, which employ a number of routinely used pharmaceutical carriers, may be used but are only representative of the many possible systems envisioned for administering compositions in accordance with the invention.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's).

Other injectable drug delivery systems include solutions, suspensions, gels. Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

The compounds used in the method of the present invention may be in a salt form. As used herein, a “salt” is a salt of the instant compounds which has been modified by making acid or base salts of the compounds. In the case of compounds used to treat an infection or disease, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The present invention includes esters or pharmaceutically acceptable esters of the compounds of the present method. The term “ester” includes, but is not limited to, a compound containing the R—CO—OR′ group. The “R—CO—O” portion may be derived from the parent compound of the present invention. The “R′” portion includes, but is not limited to, alkyl, alkenyl, alkynyl, heteroalkyl, aryl, and carboxy alkyl groups.

The present invention includes pharmaceutically acceptable prodrug esters of the compound of the present method. Pharmaceutically acceptable prodrug esters of the compounds of the present invention are ester derivatives which are convertible by solvolysis or under physiological conditions to the free carboxylic acids of the parent compound. An example of a pro-drug is an alkyl ester which is cleaved in vivo to yield the compound of interest.

Except where otherwise specified, when the structure of a compound used in the method of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, N Y, 1981. For example, the resolution may be carried out by preparative chromatography on a chiral column.

The compound, or salt, zwitterion, or ester thereof, is optionally provided in a pharmaceutically acceptable composition including the appropriate pharmaceutically acceptable carriers.

As used herein, an “amount” or “dose” of an agent measured in milligrams refers to the milligrams of agent present in a drug product, regardless of the form of the drug product.

As used herein, the term “therapeutically effective amount” or “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

Where a range is given in the specification it is understood that the range includes all integers within that range, and any sub-range thereof. For example, a range of 77 to 90% is a disclosure of 77, 78, 79, 80, and 81% etc.

As used herein, the terms “about” or “approximately” have the meaning of within 20% of a given value or range. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

For the foregoing embodiments, each embodiment disclosed herein is contemplated as being applicable to each of the other disclosed embodiments. Thus, all combinations of the various elements described herein are within the scope of the invention.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXEMPLIFICATION

### Example 1. Protein Phosphatase 2A as a Therapeutic Target in Small Cell Lung Cancer

In the present study, the effect of pharmacologically inhibiting PP2A with LB100, and LB100/carboplatin in SCLC was investigated employing in vitro and in vivo models. Furthermore, the effect of LB100 in combination with immunotherapy on the morphology and integrity of 3D spheroids generated using SCLC cells was also examined. Taken together, the results demonstrate that the anti-tumor effect of chemotherapeutic drugs can be enhanced by blocking PP2A with LB100 by itself or in combination with chemo and immunotherapy in SCLC.

### Results:

PP2A is Upregulated in SCLC Tumor Tissue and Cell Lines and Knocking Down PP2A Significantly Attenuates Proliferation of these Cells.

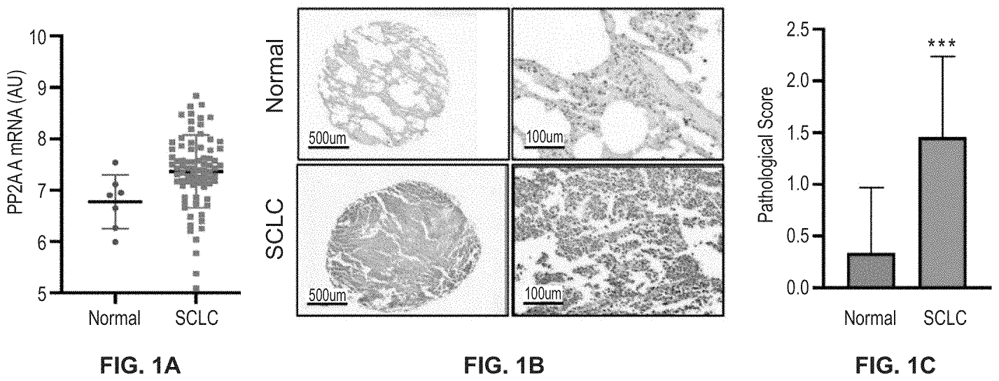

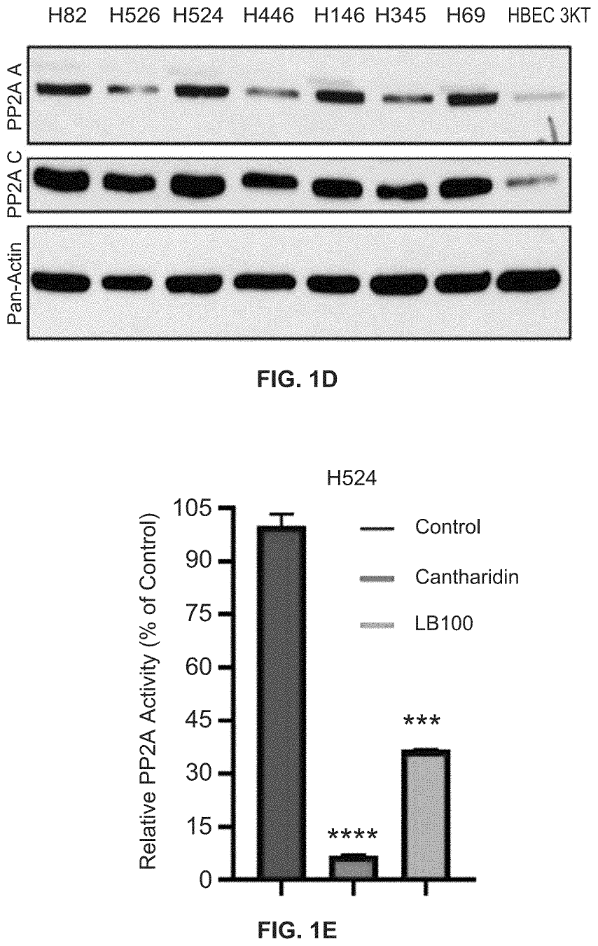

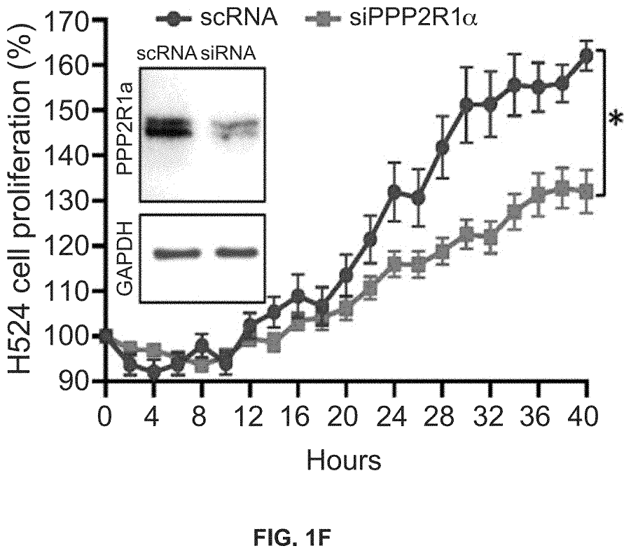

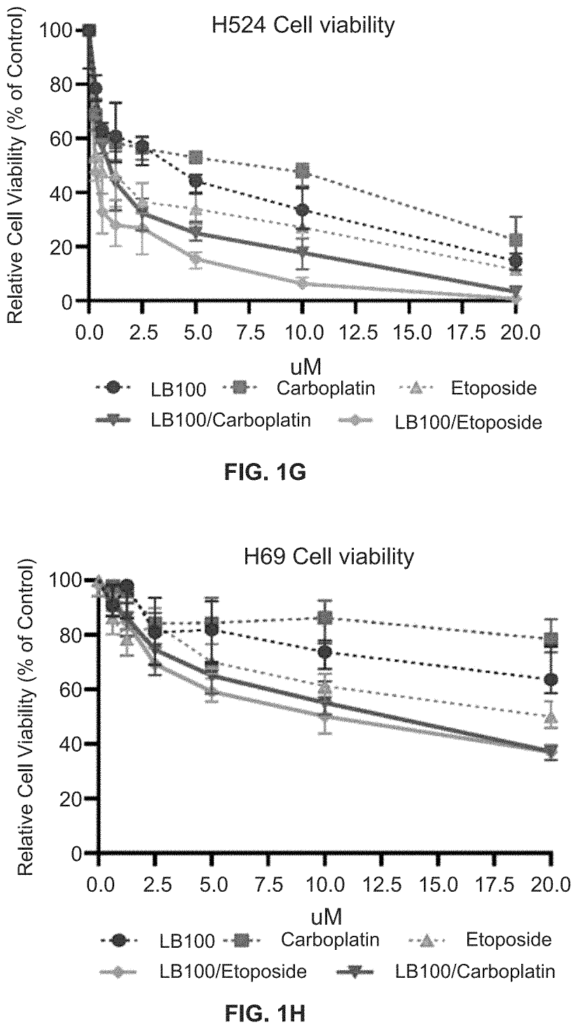

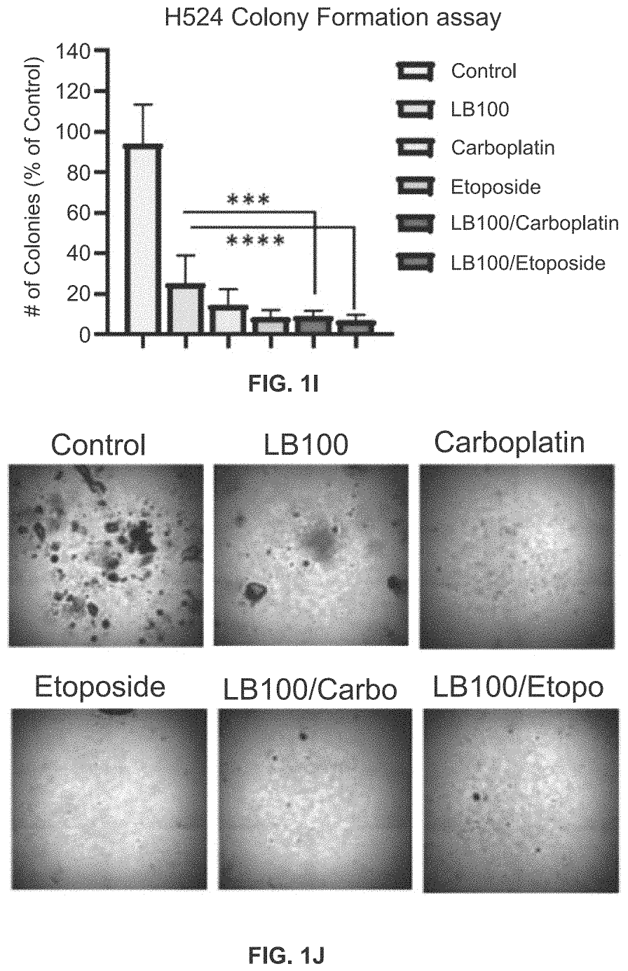

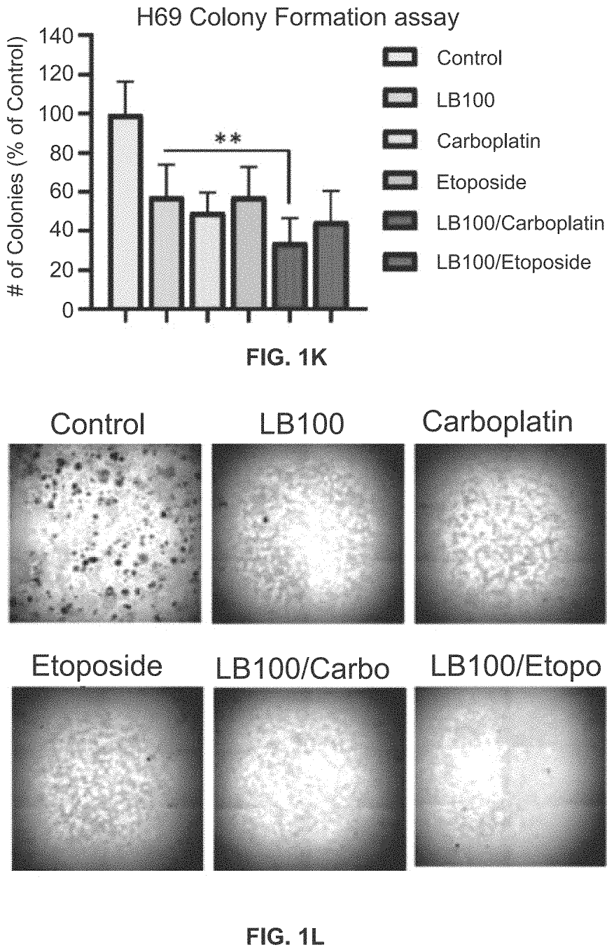

It was previously reported that PP2A and its subunits A (PP2A-A) and C (PP2A-C) are overexpressed in several SCLC cell lines (5). This was further confirmed by a bioinformatics analysis of a GEO (https://www.ncbi.nlm.nih.gov/pubmed/27093186) dataset (GSE60052), wherein PP2A-A was significantly overexpressed (p=0.0144) in SCLC as compared to normal lung (FIG. 1A).

To evaluate the expression levels of PP2A in SCLC we compared adjacent normal (n=24) and primary SCLC tumor (n=79) cores contained within tissue microarrays (TMAs) subjected to immunohistochemistry (IHC) using an antibody specific to PP2A-A (FIG. 1B). Each tumor and normal core contained in the TMA was scored independently by a pathologist who was blinded to the identity of the tissue (20, 21). PP2A-A protein was undetectable in most normal cores (0=79.17%, 1=16.67%, 2=4.16%) but was significantly upregulated in tumor tissue (0=8.86%, 1=41.77, 2=40.5, 3=8.87) (FIG. 1C). The mean pathological score for PP2A in tumor tissues (1.45±0.088) was significantly higher (p=0.001) than that of normal tissues (0.333±0.13). Both the publicly available data sets and TMA results showed that PP2A-A expression was significantly upregulated in the SCLC tumor tissue (FIGS. 1A to 1C). Having confirmed overexpression in tumor tissue, we next determined their expression in various SCLC cell lines by immunoblotting, as described previously (22). Both subunits were upregulated in SCLC cell lines including H82, H526, H524, H446, H146, H345 and H69 compared to control HBEC 3KT cells (FIG. 1D).

Cantharidin is the parent compound of LB100 that is known to inhibit PP2A. Therefore, we used cantharidin as a positive control to demonstrate that inhibiting PP2A results in the observed effects in SCLC cells. Indeed, cantharidin treatment reduced PP2A activity by almost 90% while LB100 significantly inhibited phosphatase activity to 65%. (FIG. 1E). Finally, we knocked down PP2A subunit Aα using a specific siRNA in H524 SCLC cells. A scrambled version (scRNA) was used as control. As expected, knocking down PP2A significantly decreased PP2A subunit Aα level and attenuated cellular proliferation in these cells (FIG. 1F).

Combining Chemotherapy with LB100 Resulted in Synergy.

To test the cytotoxicity effect of LB100, carboplatin and etoposide, we treated six SCLC cell lines with various concentrations of each drug for 72 hours. In four cell lines H82, H526, H524 and H446 that were sensitive to cisplatin, LB100 induced cell death more effectively with an IC50 of <8 μM (Table A) compared to the two other cell lines H146 and H69 that were resistant to cisplatin in which cell death was observed at relatively higher doses of LB100 (IC50˜20 μM).

TABLE A
Cytotoxicity IC50 values of SCLC cell lines

| Cell line | LB100 (μM) | Carboplatin (μM) | Etoposide (μM) |
|---|---|---|---|
| H82 | 3.5 + 3 | 46.5 + 6.8 | 22.6 + 6.3 |
| H526 | 7.2 + 2.8 | 33.2 | 2.8 + 0.8 |
| H524 | 5.3 + 3.2 | 8.2 + 2.6 | 3 + 2.4 |
| H446 | 6.9 + 3.6 | 26.2 + 3.8 | 3 + 1.8 |
| H146 | R | 8.3 + 4.8 | 31.2 |
| H69 | 22.6 + 5.1 | R | 30 + 3.3 |

Next, we determined the effect of treating SCLC cell lines with combinations of LB100 and the chemotherapeutic drugs agents, carboplatin and etoposide. Either drug alone was effective in killing H524 SCLC cells that are sensitive to LB100 (FIG. 1G). However, cell death was significantly higher when LB100 was used in combination with carboplatin or etoposide with combination index (CI) values of 0.534 and 0.532 respectively (FIG. 1G). A similar synergy was seen in the case of H69 SCLC cells. LB100/carboplatin and LB100/etoposide killed LB100-resistant H69 cells with CI values of 0.311 and CI=0.646, respectively (FIG. 1H).

To determine the cytotoxicity effect of LB100 alone and in combination with carboplatin and etoposide on H524 and H69 cells, we also performed colony formation assays. Treatment with single drug (LB100, carboplatin or etoposide) or in combination (LB100/carboplatin and LB100/etoposide) significantly reduced colony formation in both cell lines (p<0.0001; p<0.01) (FIGS. 1I-1L). While colony formation by H524 cells was dramatically reduced compared with LB100 single treatment in both drug combination groups (LB100/carboplatin and LB100/etoposide). However, in the case of the H69 cells, a significant difference was observed only between LB100 and LB100/carboplatin treated cells (FIGS. 1K and 1L). Therefore, we investigated the effect of LB100 using a 3D cell culture model that resembles the tumor microenvironment more closely.

The Effect of LB100 on H446 Spheroid Growth was Tested.

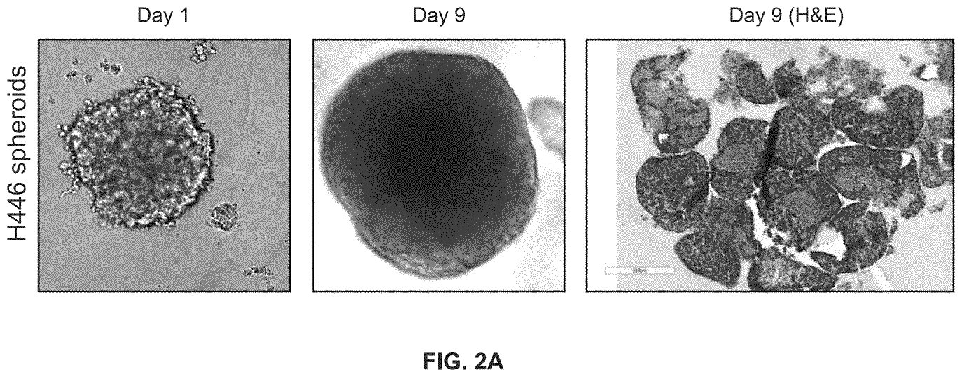

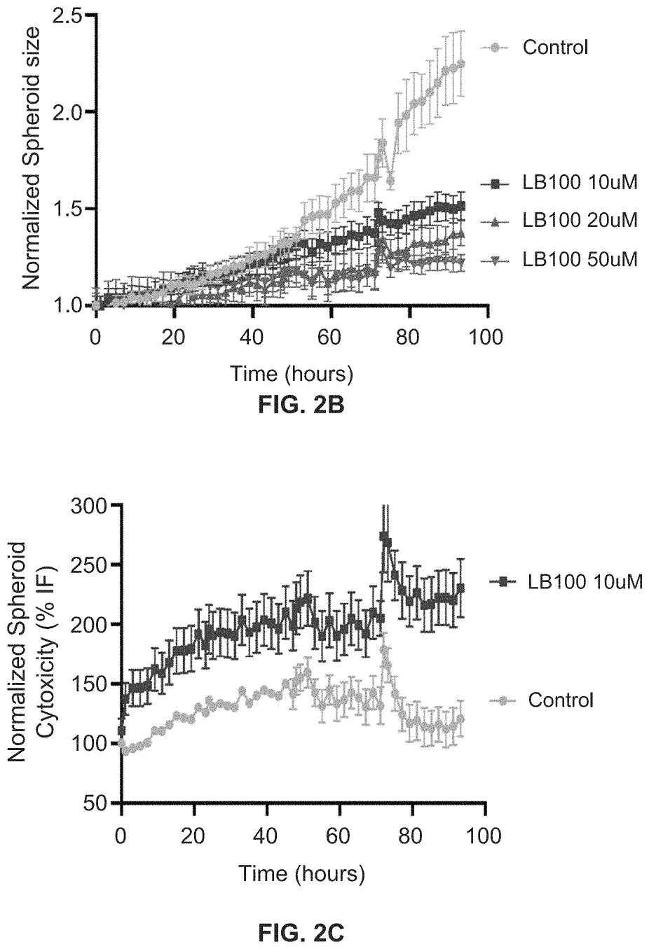

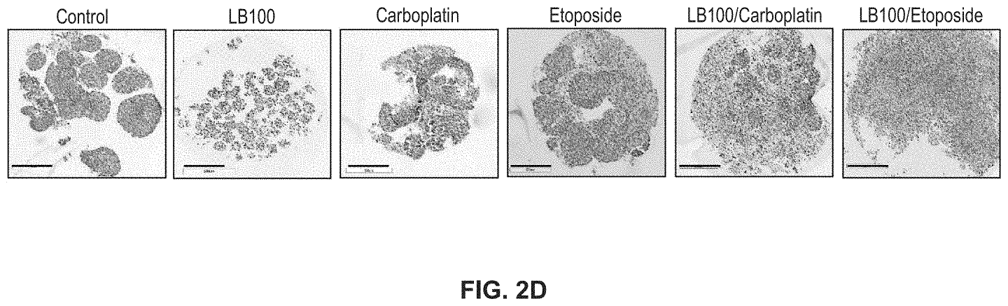

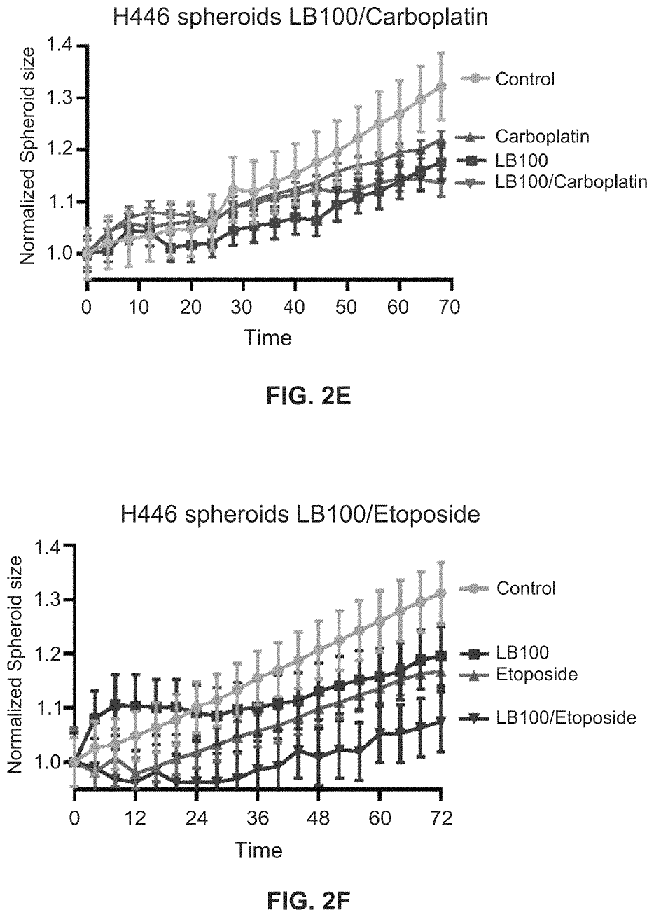

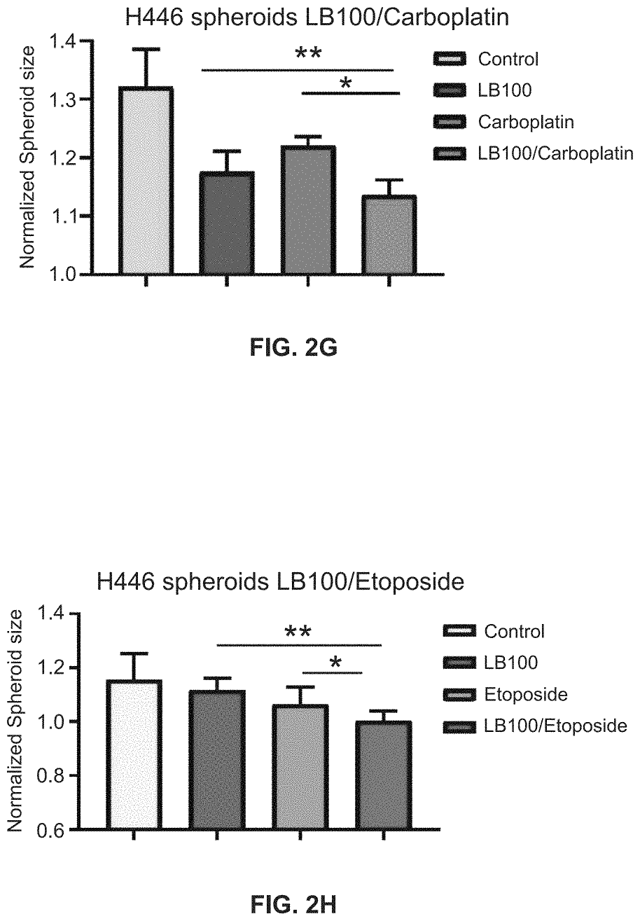

We further investigated the effect of LB100 and the chemotherapy drugs on spheroids formed by SCLC cells. Three cell lines H524, H69 and H446 were tested. The H524 and H69 cells formed large soft clumps in low-attachment 96 well plates. H446 cells that formed dense spheroids overnight without the addition of extracellular matrix components or matrigel were used for imaging and histological analysis. Spheroids of 300-500 μm formed in nine days (FIG. 2A) and the size of the spheroids formed in vitro was comparable to the tumors formed in metastatic sites where the cells experience conditions of hypoxia, inflammation, changes in pH levels and often, nutrient deprivation (23). To test the effect of LB100 on H446 spheroids, we used the IncuCyte Live-Cell Analysis System to record functional changes in real time. H446 spheroids treated with or without 20 μM LB100 were imaged in brightfield (BF) and using green fluorescence over 72 hours. The size of the spheroids was measured using an automated software algorithm that masked the largest BF in the field of view (label-free, real-time live cell assay for spheroids: IncuCyte bright-field analysis). BF analysis illustrated spheroid shrinkage and increase in the cytotoxicity dye fluorescence after LB100 treatment (FIGS. 2B and 2C). H&E staining was performed on spheroids treated with LB100, carboplatin alone, and in combination. Before treatment, spheroids had a dense, round shape (FIG. 2D—Control) with very well-defined contours. However, 72-hour of treatment with LB100, carboplatin, etoposide or combination of chemotherapeutic drugs with LB100 significantly changed the morphology of spheroids. Spheroids decreased in size and lost their round shape with LB100 treatment. Carboplatin and etoposide treatments dissociated cells from spheroids, forming diffuse clouds of cells around them. Drug combination of carboplatin or etoposide with LB100 abolished spheroid growth and notably decreased the number of spheroids (FIG. 2D). IncuCyte BF analysis on H446 spheroid growth demonstrated that LB100 in combination with carboplatin reduced single spheroid size compared to control or only LB100 treatment (FIGS. 2E and 2G). Similar results were obtained with LB100 and etoposide (FIGS. 2F and 2H). These results confirmed the efficacy of LB100 alone and in combination with carboplatin or etoposide in the 3D spheroid model, similar to our observations in 2D cultures.

Drug Combination Inhibited SCLC Cell Invasion, Increased Carboplatin Uptake, and Affected PP2A, DNA Damage and Apoptosis Regulatory Proteins.

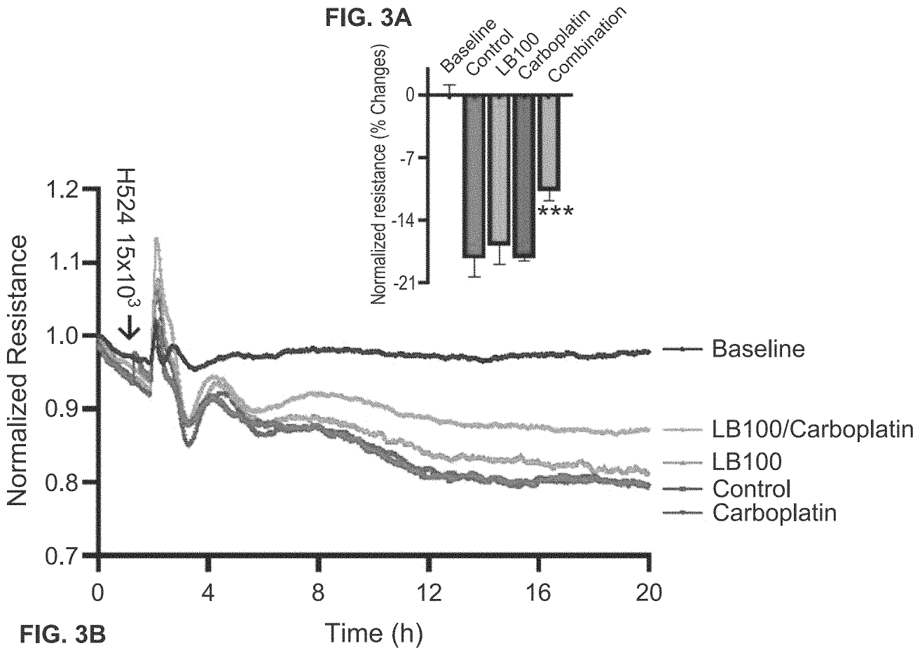

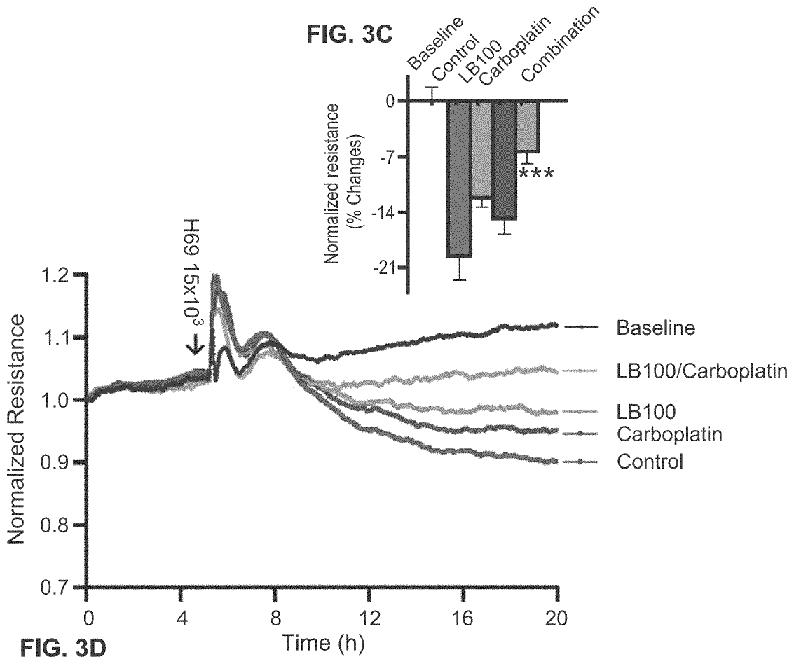

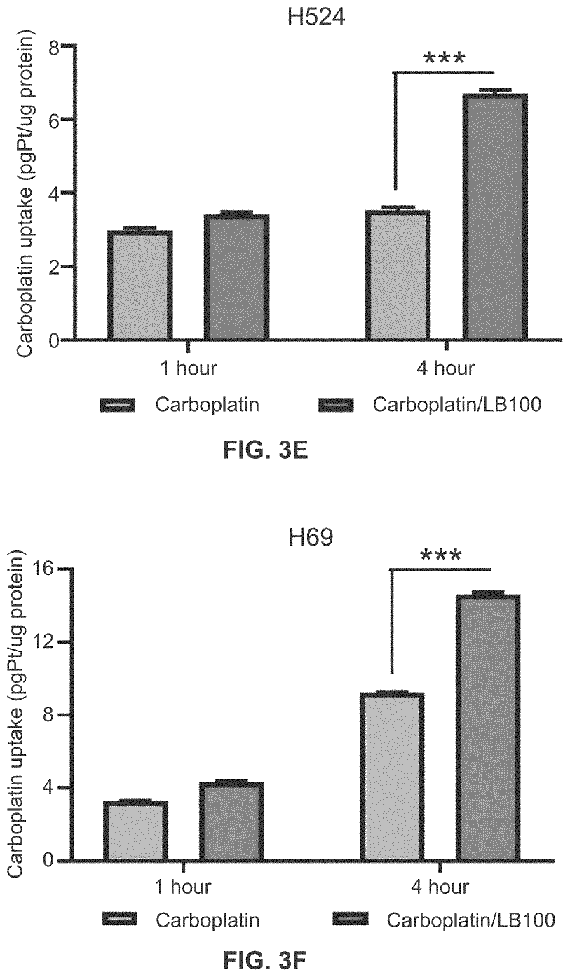

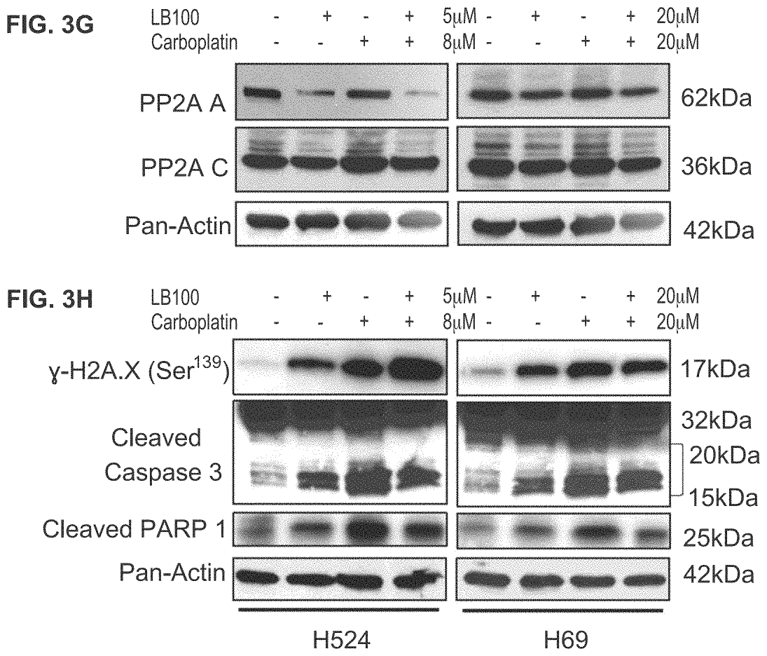

To discern the effect of LB100 on cell invasion, we tested the ability of SCLC cells to invade though a layer of endothelial cells (ECs). Toward this end, we measured the trans-endothelial monolayer resistance using an electrical substrate-impedance sensing system (Applied Biophysics, Troy, N.Y., USA), as previously described (24). This system continuously measures endothelial monolayer resistance as SCLC cells attach and begin to invade into the monolayer. A decrease in resistance indicates a disrupted endothelial monolayer barrier via trans-endothelial extravasation of tumor cells. Untreated control cells highly invaded through HUVEC monolayer. After single drug treatments (LB100 or carboplatin), H524 cells showed no changes in transmigration ability (% change control=18.2+2; LB100=16.9+2; carboplatin=18.2+0.4) and for H69 cells the corresponding values were control=19.6+1.7; LB100=12.3+0.92; carboplatin=14.9+1.24 (FIGS. 3A-3D). However, drug combination treatment significantly reduced cell transmigration ability through HUVEC monolayer as compared to untreated control cells (p<0.001). Inserts indicate a lower percent change of HUVEC barrier disruption for H524 (10.6+1.2%) and H69 (6.6+1.2%) after 20 hours of LB100+ carboplatin treatment (p<0.001). This suggests that combinatory inhibition of PP2A with chemotherapy could potentially disrupt cell motility through vessels and prevent invasion.

Since a combination of LB100 and carboplatin or etoposide showed a synergistic effect, we wished to discern the mechanism by which the drugs worked synergistically. To this end, platinum (Pt) levels were measured in H524 and H69 cells using Inductively Coupled Plasma Mass Spectrometry (ICP-MS). Cells were pretreated with LB100 for 24 hours with subsequent 5 μM (H524 cells) and 20 μM (H69 cells) treatments of carboplatin for 1 or 4 hours. Treating cells for 1 hour with carboplatin only mildly elevated level of Pt in both cell lines relative to the control (FIGS. 3E and 3F). The 4-hour treatment with drug combination significantly increased the level of Pt in both cell lines compared with single treatment of carboplatin alone, suggesting that LB100 enhanced the uptake of Pt in SCLC cells and thus, promoted the pro-apoptotic effect of carboplatin.

We examined the effects of LB100 alone and in combination with carboplatin on the expression of PP2A. The drug treatment drastically reduced the expression of PP2A subunit A in H524 cells (FIG. 3G, left upper panel). But in the case of the H69 cells, subunit A expression was the same for control and treated cells (FIG. 3G, right upper panel). The expression of subunit C was also unchanged in the control and treated H524 and H69 cells (FIG. 3G, middle panel). Moreover, LB100, carboplatin and combination therapy significantly affected phosphorylation of histone γ-H2AX, the marker that correlates with DNA damage and induction of apoptosis in H524 and H69 cells (FIG. 3H). Additionally, caspase 3 was activated in H524 and H69 cells after single treatment with LB100 or carboplatin, as well as in combination, as seen by cleavage of the preform (FIG. 3H). Moreover, the dysregulation of PP2A induced PARP activity, leading to cell death. Together, these data demonstrated that inhibition of PP2A by LB100 in combination with platinum drugs induced apoptotic signaling in SCLC cells.

The Effect of LB100 on the Kinomics Profile of H524 Cells was Explored.

Since LB100 selectively inhibits PP2A, we used PamGene technology to detect the phosphorylation of peptides as a functional readout of the cellular serine/threonine kinases (STKs). This analysis allowed us to interrogate the inhibitory effect of LB100 on protein phosphorylation throughout a variety of cellular pathways. It was found that LB100, at 5 and 10 μM concentrations significantly increased the phosphorylation of certain STKs (n=20). Surprisingly, treatment of H524 cells with 5 μM and 10 μM of LB100 significantly reduced the tyrosine kinase peptide phosphorylation (n=52).

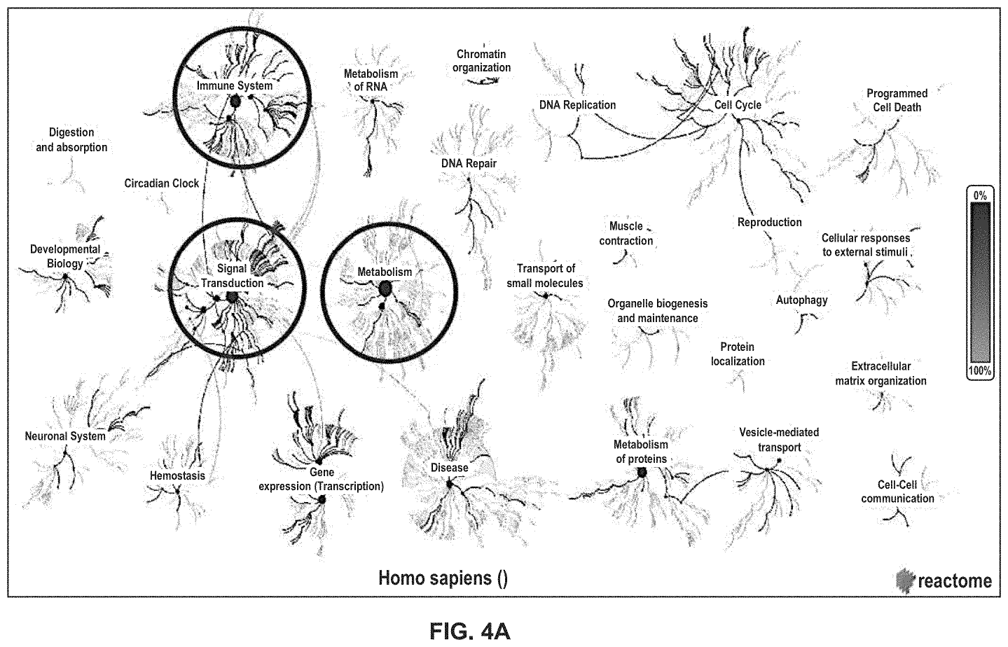

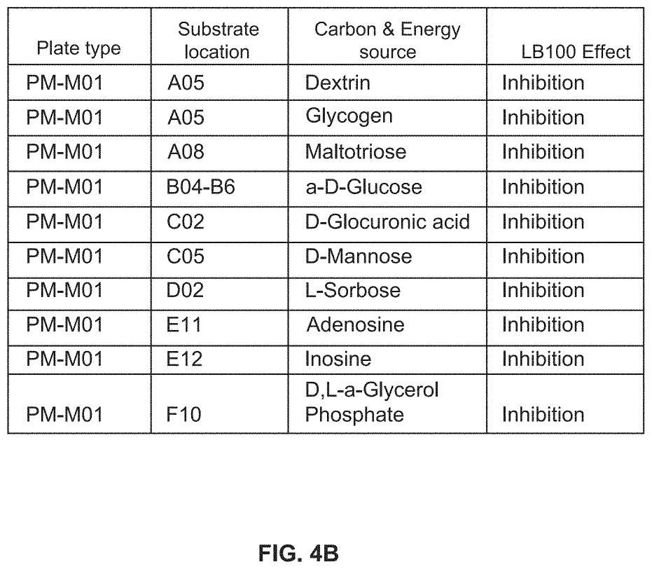

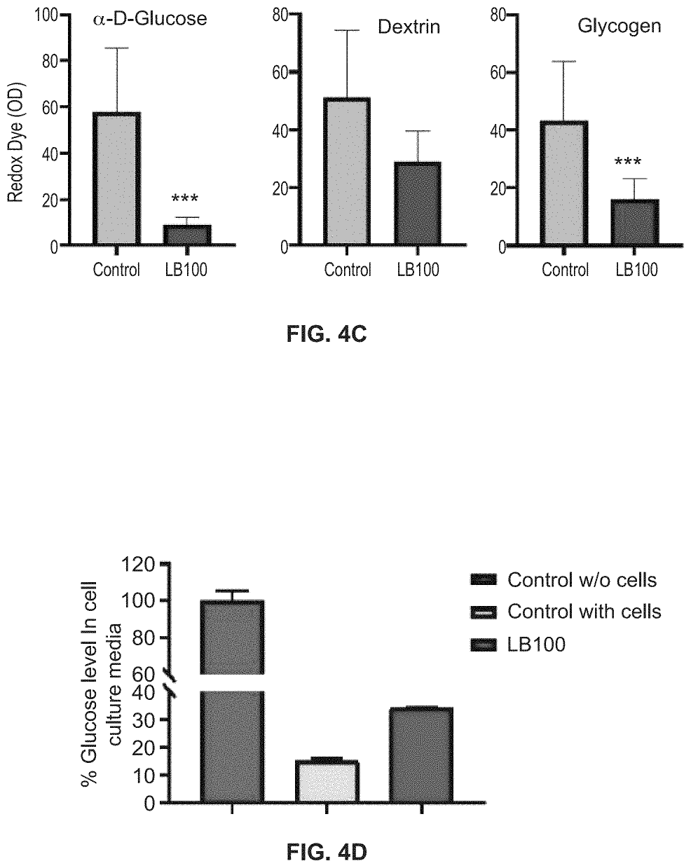

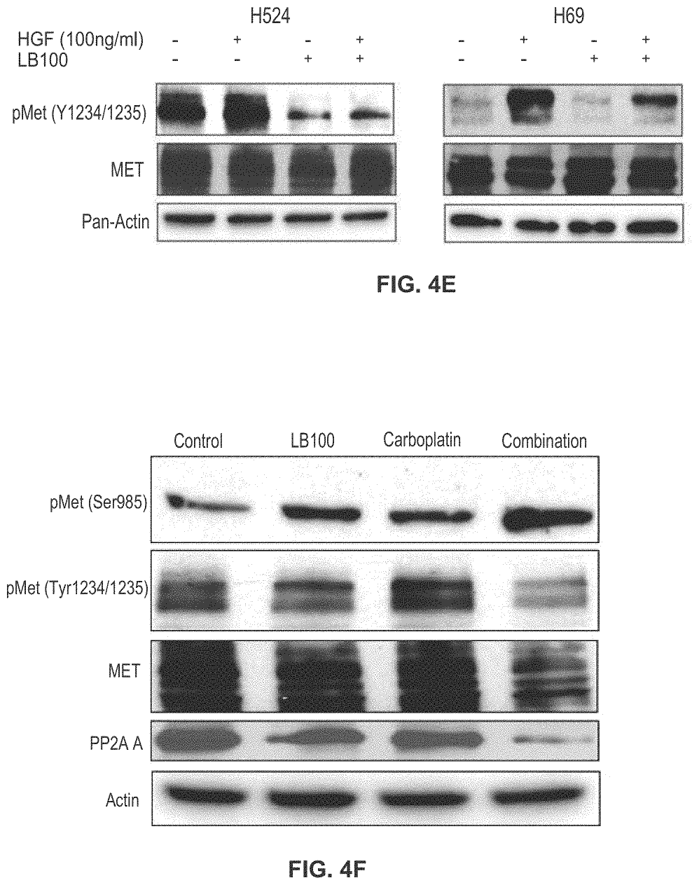

A bioinformatics analysis using the Reactome software for enrichment analysis revealed that several pathways were selected as particularly interesting based on a priori knowledge of the effect of LB100 on tumorigenesis (27-30). LB100-mediated inhibition of PP2A strongly influenced both signal transduction and metabolic pathways (FIG. 4A). A closer analysis of the signal transduction pathway showed that, consistent with previous reports (31, 32), LB100 affected HGF-MET signaling. In addition, LB100 also targeted metabolic signaling in SCLC cells.

The Effect of LB100 on Metabolic Pathways in H69 Cells was Explored.

To discern the effect of LB100 on metabolic signaling, we examined the utilization of carbon sources by H69 employing BiOLOG (Hayward, Calif.) Phenotype Microarray technology. Using this assay, we examined 94 carbon sources and the redox dye tetrazolium to detect substrate utilization. LB100 inhibited the utilization of 11 carbon substrates compared to control (untreated) H69 cells (FIG. 4B) that could be divided into five groups: sugars (L-sorbose, α-D-Glucose, D-Mannose), polysaccharides (glycogen, D-Glucuronic acid), carbohydrates (dextrin, maltotriose), phosphorylated compounds (D,L-α-Glycerol Phosphate) and amines (adenosine, inosine). Of these, the consumption of three substrates important for anabolic biosynthetic reactions namely, α-D-Glucose (more than 6-fold) and glycogen (more than 2.7-fold) was significantly reduced after LB100 treatment in H69 cells (FIG. 4C). Additionally, LB100 inhibited adenosine and inosine substrate utilization in these cells that could have a significant effect on purinergic signaling in SCLC. Finally, glucose uptake from cell culture media by H69 cells was measured directly using a Glucose Oxidase Assay and, as expected, was found to be reduced upon treatment with LB100. The Glucose level in control media with cells was less than 20% of the control without cells (100%). LB100 treatment reduced the consumption of glucose in media by 65% compared with control without cells (FIG. 4D).

The Effect of LB100 on MET Phosphorylation in H524 and, H69 Cells was Explored.

The PamGene kinomic data showed decreased MET peptide phosphorylation between residues 1227 and 1239. To validate this finding, we performed western blotting experiments with H524 and H69 cell extracts, following treatment with LB100 (5 μM and 20 μM, respectively), and stimulation with HGF for 10 min using a Phospho-MET (pMET) antibody that specifically detects phosphorylated tyrosine 1234/1235. Pretreatment of the H524 cells with LB100 almost abrogated MET basal and HGF activated phosphorylation of MET (FIG. 4E, left panel). In H69 cells the level of HGF phosphorylation significantly decreased (FIG. 4E, right panel) suggesting that inhibiting PP2A with LB100 the affects HGF/MET signaling responsible for cell viability, proliferation and motility.

Previous studies demonstrated that Ser985 phosphorylation of MET negatively regulated MET kinase activity (33-35). Our results also showed that treatment of H524 cells with LB100 or in combination with carboplatin induced increase in Ser985 phosphorylation and was related with inhibition of MET tyrosine phosphorylation. Moreover, LB100 reduced the expression of PP2A A in LB100/carboplatin samples (FIG. 4F). This finding correlates with PamGene kinomic data that LB100 reduced the Tyr 1234/1235 MET phosphorylation and can be key effect of LB100 on SCLC cells.

The Effect of LB100 on Mitochondrial and Glycolytic Function of SCLC Cells was Explored.

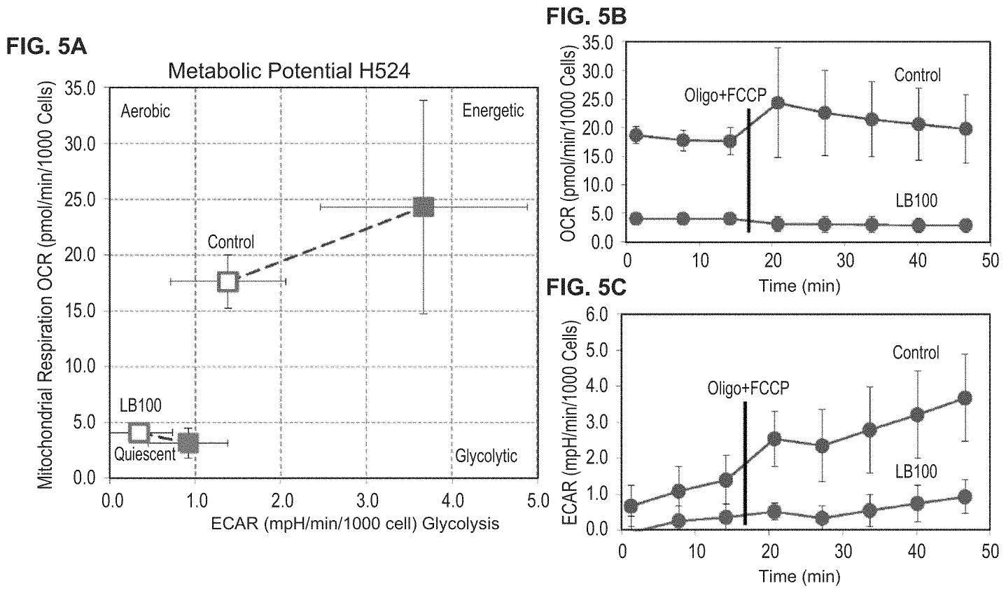

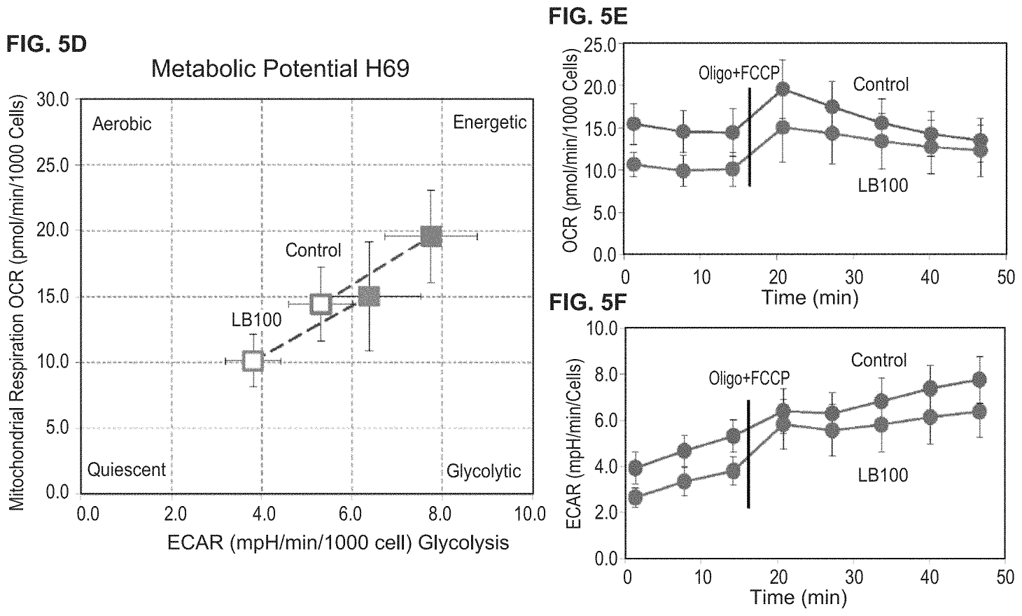

Next, we determined the effect of LB100 on ATP production in SCLC cells employing the Seahorse XF Cell Energy Phenotype Test. H524 and H69 cells were pretreated with half the IC50 dose of LB100 (2.5 μM and 10 μM, respectively). After drug treatment, we counted the number of cells and examined them for viability using exclusion of trypan blue as a readout. Cellular basal oxygen consumption rate (OCR) and extra-cellular acidification rate (ECAR) measurements were determined on a Seahorse XF96 analyzer. H524 and H69 cells were then stressed with a combination of 1 μM of oligomycin (inhibitor of oxidative phosphorylation (OxPhos) and 1 μM carbonyl cyanide p-trifluoromethoxy-phenylhydrazone (FCCP) (an uncoupler of OxPhos). Since oligomycin inhibits mitochondrial ATP production and FCCP induces maximum oxygen consumption by uncoupling the H+ gradient in mitochondria, the experimental conditions examined with these two stressed methods reflect the maximum glycolytic capacity and OxPhos capacity of SCLC cells, respectively. Cellular metabolic capacity includes both events and characterizes the limit of cell to acute increases in energy demands. LB100 severely affected energy metabolism of H524 cells; and their basal OCR was 4-fold lower compared to untreated cells (FIG. 5A). LB100 treatment also induced inhibition of stressed OCR as well as basal and stressed ECAR (FIGS. 5B and 5C). These results demonstrated a significant repressive effect of LB100 on glycolytic and OxPhos pathways, the major sources of ATP production in these cells. A significant decrease in basal OCR and ECAR was also observed in H69 cells (FIG. 5D). However, there was no significant reduction of stressed OCR and ECAR in these cells upon treatment with LB100 (FIGS. 5E and 5F).

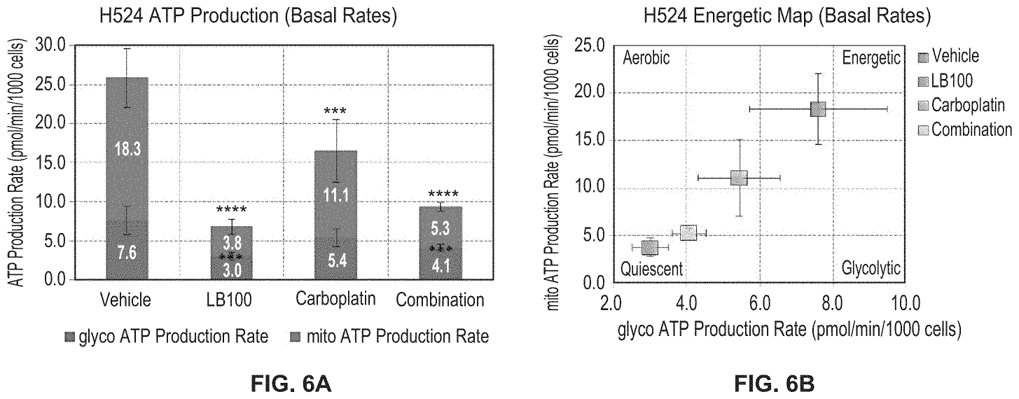

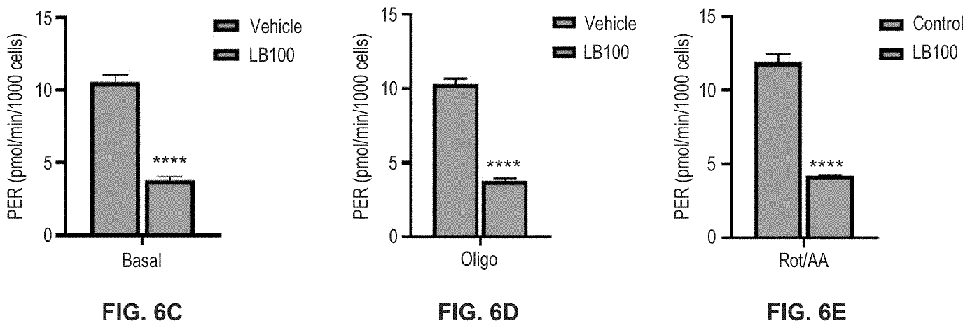

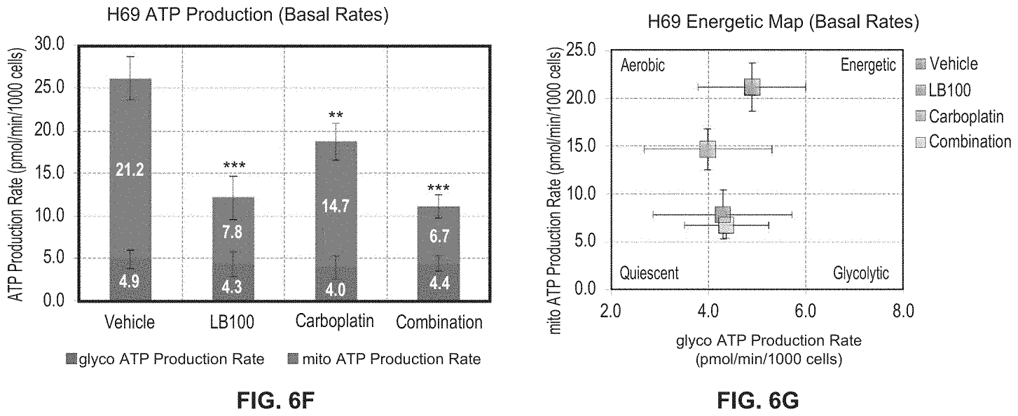

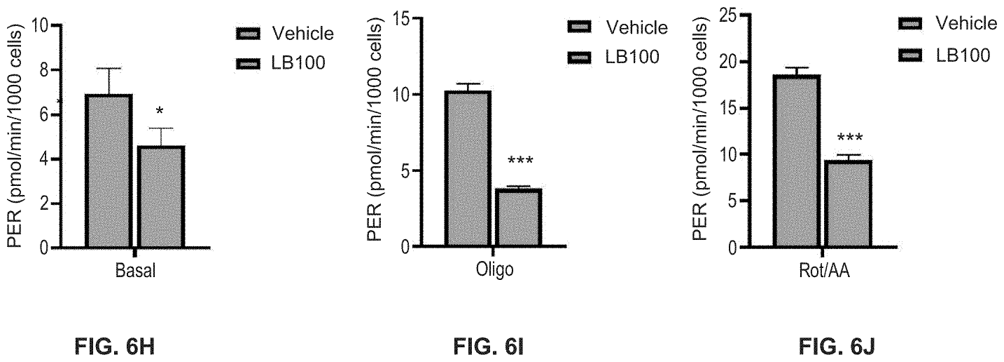

To determine the role of LB100 alone or in combination with carboplatin on ATP production from mitochondrial respiration and glycolysis, we performed an Agilent Seahorse XF-96 Real-Time ATP rate assay. In H524 cells, total ATP production rate was significantly reduced in all three groups compared to untreated cells by 73.7% (LB100), 36.3% (carboplatin) and 63.7% (LB100/carboplatin) (FIG. 6A). Mitochondrial and glycolytic ATP production rates were also significantly lower in drug-treated cells. Importantly, LB100 and LB100/carboplatin were more effective in inhibiting mitochondrial ATP and glycolytic ATP production than carboplatin alone and changed energetic phenotype of H524 cells. The cells tended to become less energetic and glycolytic (FIG. 6B).

To elucidate the effect of the drugs on the glycolytic metabolism of H524 cells, we analyzed the proton efflux rate (PER). PER is calculated by subtracting acidification produced from mitochondrial CO2 production (Mitochondrial-derived CO₂ can partially hydrate in the extracellular medium, resulting in additional extracellular acidification beyond that contributed by glycolysis) from total acidification or protons efflux (from both glycolysis and mitochondrial) into the extra cellular medium. Basal values of the PER were reduced by >50% upon drug treatment compared to untreated cells (FIG. 6C). Measurement of the PER in the presence of oligomycin, an inhibitor of OxPhos, and a second acute injection of antimycin/rotenon (inhibitors of mitochondrial electron transport), showed a significant decrease in LB100 treated group. LB100 treatment also impaired glycolysis and reduced compensatory glycolysis (the ability of the cells to increase glycolysis after OxPhos inhibition with antimycin/rotenone) (FIGS. 6D and 6E). Additionally, measurements of ATP production in H69 cells. H69 cells showed the same trend as H524 cells in that, the total ATP production rate dropped by 54% in LB100 group, by 12% in carboplatin group and 57% in the LB100/carboplatin group (FIG. 6F). Moreover, LB100 and LB100/carboplatin significantly reduced mitochondrial ATP production rate in H69 cells and the energetic map of H69 cells showed that the glycolytic ATP production rate dropped slightly in comparison with untreated cells (FIG. 6G). To confirm that LB100 also affected glycolytic pathway in LB100-resistant cells, we measured PER in these cells. Basal level of PER was significantly inhibited in LB100 group (FIG. 6H). In addition, LB100 treatment significantly inhibited PER in the presence of mitochondrial electron transport inhibitors (FIGS. 6I and 6J). LB100 alone or in combination with carboplatin led to compromised glycolytic metabolic activity and limited oxidative capacity in in H69 cells. Collectively, these results showed that LB100, alone or in combination with carboplatin effectively targeted the metabolic function of SCLC cells, thereby decreasing cell proliferation and migration, rendering them sensitive to chemotherapy.

LB100 and Atezolizumab Increased the Recognition of Tumor Cells in 3D by CD8+ T Cells.

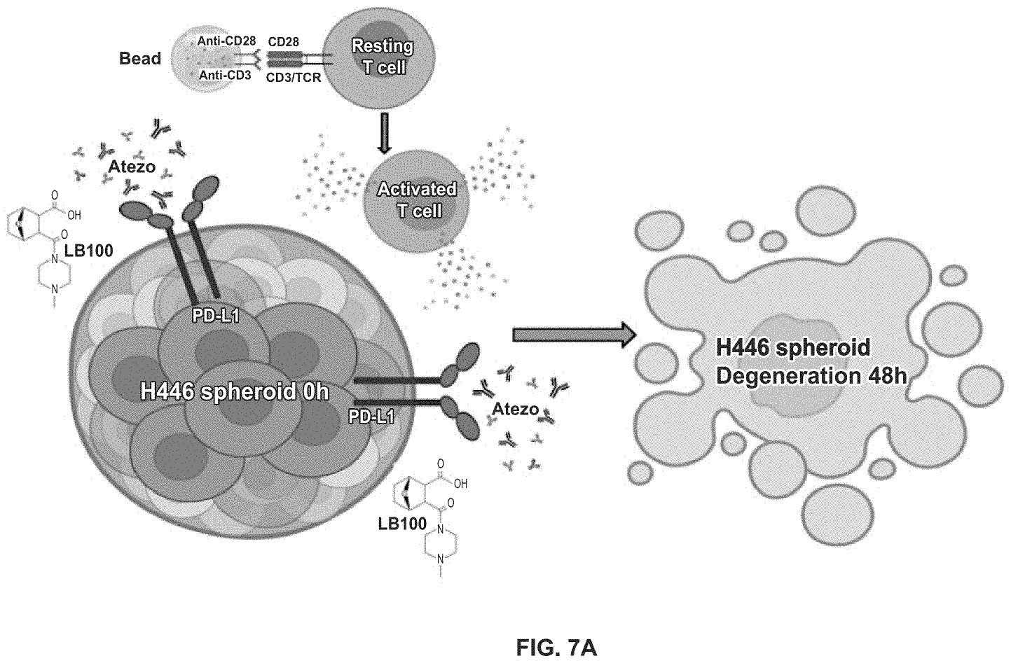

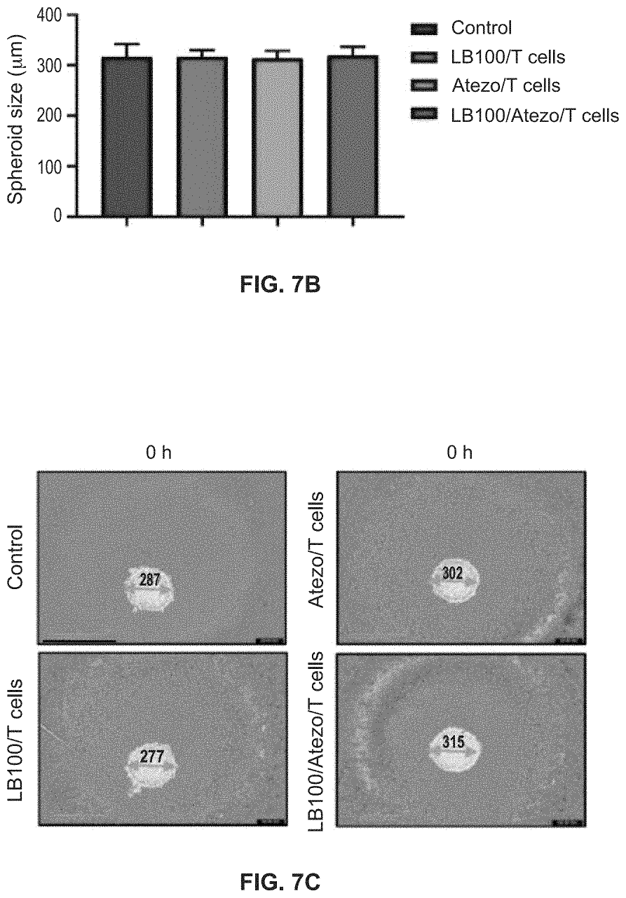

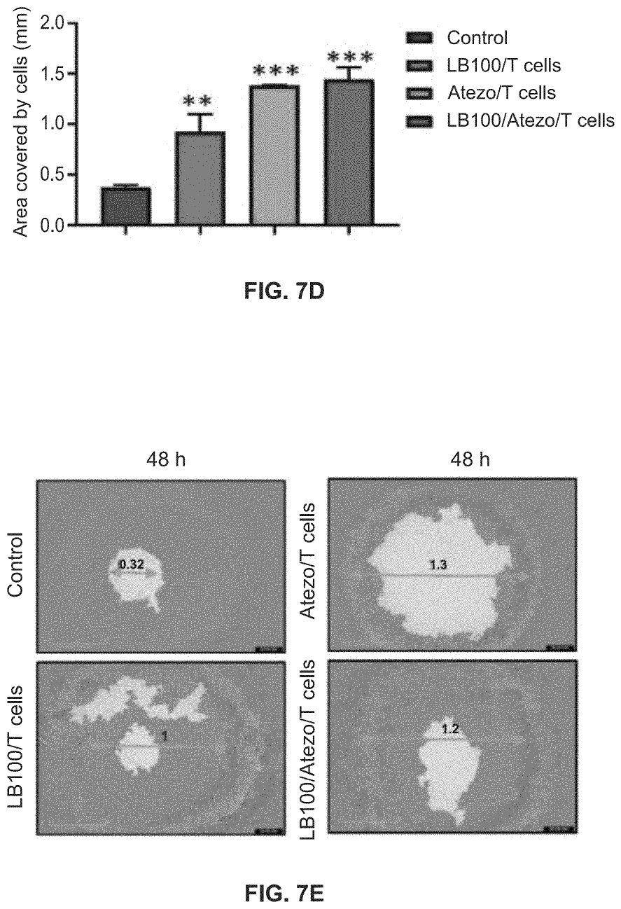

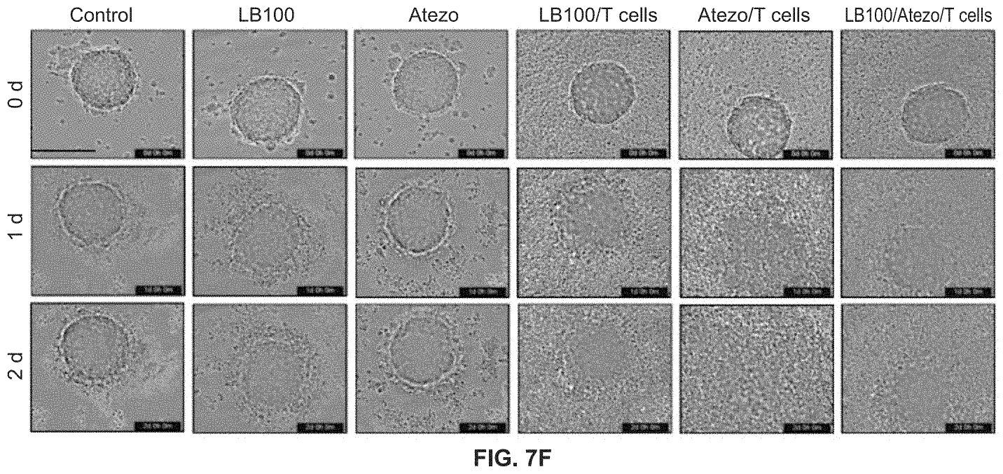

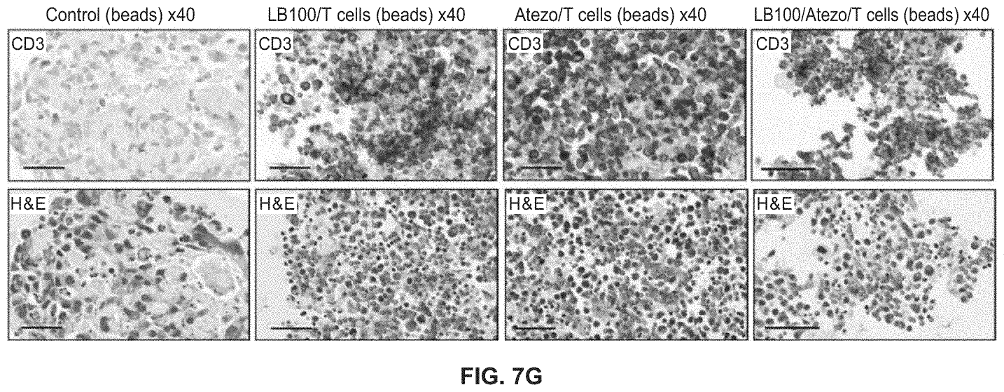

Since checkpoint inhibitors can induce an anticancer immune response and PP2A inhibition has been shown to enhance anticancer immunity in several cancers, we evaluated the combination of LB100 and atezolizumab, and a humanized IgG antibody that targets PD-L1 in a 3D culture system using H446 spheroids in the presence of T cells. Cytotoxic CD8+ cells were isolated from whole blood, buffy coat of healthy donors following the protocol described in the Methods. FIG. 7A contains a schematic showing the treatment protocol. H446 spheroids were placed in a round bottom 96 well plate with T cells and activated beads and LB100, atezolizumab or a combination of LB100 and atezolizumab and the spheroids were visualized with time-lapse imaging. The average spheroid diameter was between 300 and 350 μm and they had the same morphology at 0 hours (FIGS. 7B and 7C). Spheroid survival was monitored for 48 hours and their diameters were measured from phase contrast images. Cell distribution diameters significantly (p<0.001) increased after atezolizumab/T cells and LB100/atezolizumab/T cells groups compared to control (FIGS. 7D and 7E). LB100 alone had moderate effect (p<0.01) on spheroid degeneration (FIG. 7D). T cells in combination with LB100 or atezolizumab affected spheroid integrity. Bright field images from IncuCyte time-lapse microscopy showed that from day 0 spheroids had a round shape and well-represented spheroid structure (FIG. 7F). LB100 without T cells began disintegrating the spheroids after day 1 and atezolizumab without T cells had no effect on the spheroids. Activated T cells in combination with LB100, atezolizumab and both drugs induced shedding of dead cells, accumulation T cells in spheroid core and at day 2 only spheroid fragments were observed in the images (FIG. 7F). IHC using a CD3 antibody showed T cell clusters among the tumor cells in three groups LB100/T cells, atezolizumab/T cells and LB100/atezolizumab/T cells. Combination treatment induced the destruction of spheroids, led to infiltration of the activated T cells in the spheroids resulting in the dissociation of cells, loss of spheroid morphology and increased cell cytotoxicity. Clusters of T cells+ beads on the H&E staining matched the brown spots of CD3 staining (FIG. 7G).

The Effect of LB100 on Tumor Growth in a Mouse Model of SCLC was Explored.

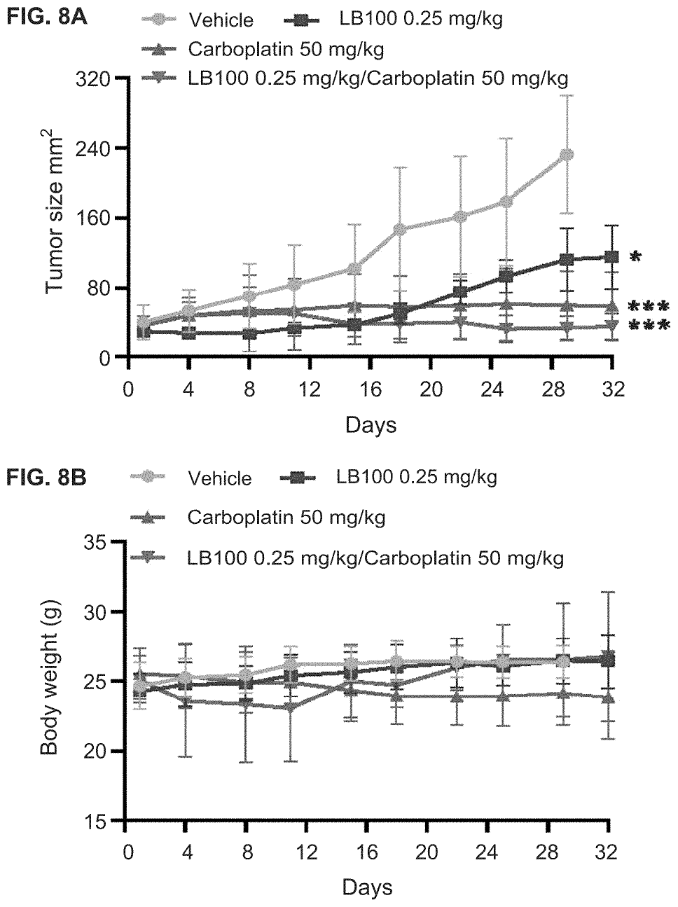

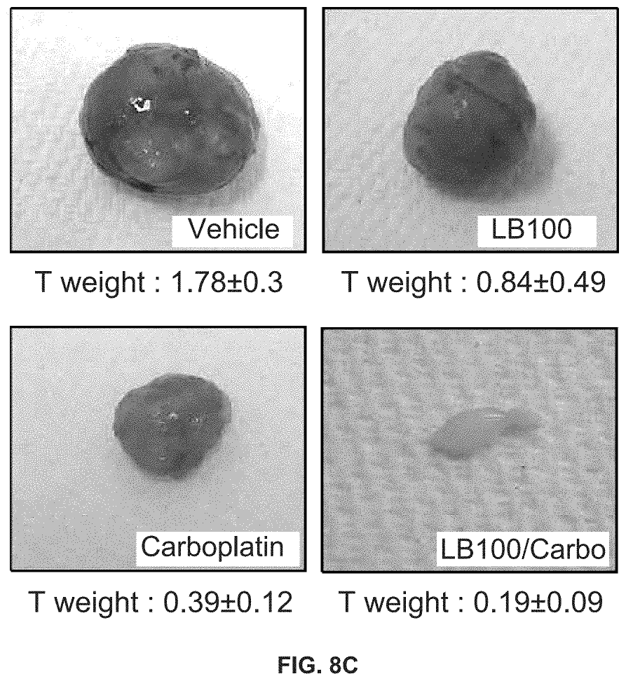

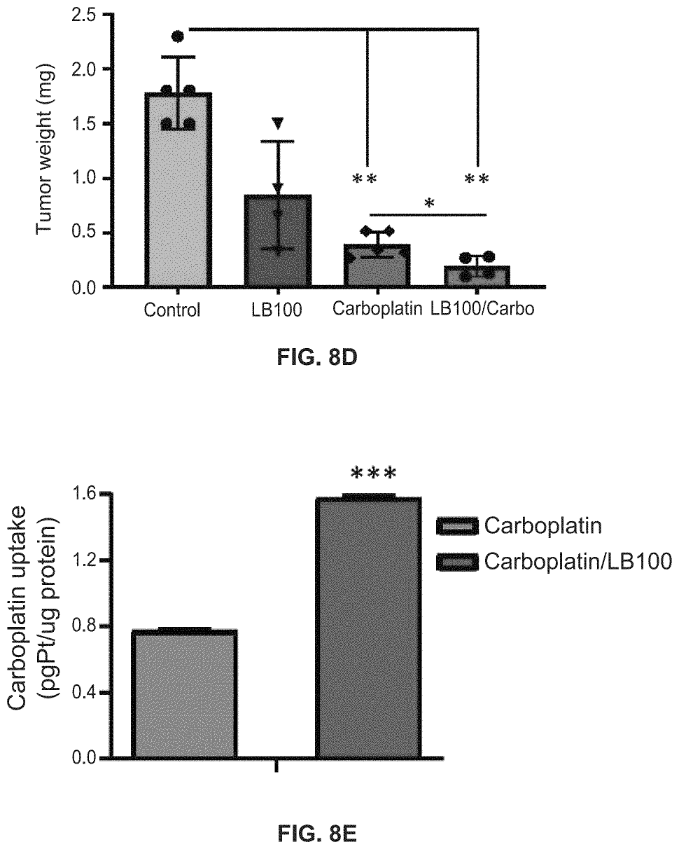

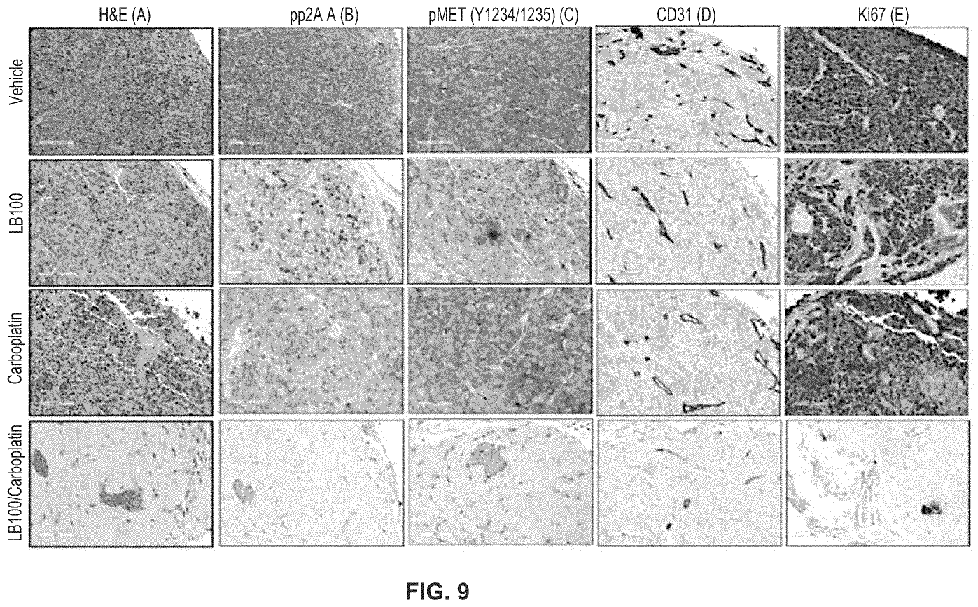

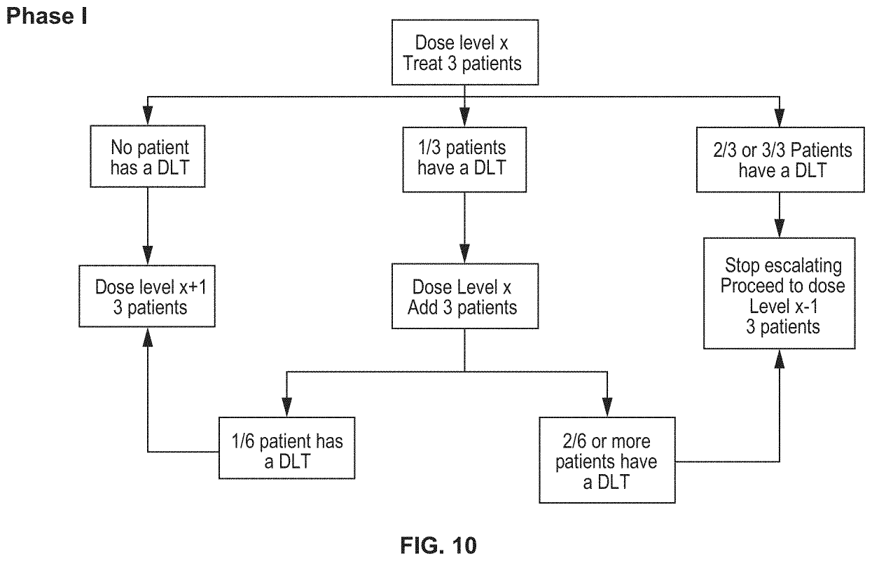

Having demonstrated the potency of LB100, carboplatin, and their combination in an in vitro system, we next examined in vivo using a xenograft mouse model of SCLC. Treatment with LB100 or a combination of LB100 and carboplatin resulted in a statistically significant reduction in tumor size (FIG. 8A). Notably, the drugs did not exhibit significant toxicity, nor did they significantly affect the body weight (FIG. 8B). However, treatment with LB100, carboplatin, and their combination, caused a significant reduction in tumor weight compared to the vehicle-treated group (FIG. 8C). LB100/carboplatin inhibited primary tumor growth by 89% compared with vehicle group. The results demonstrated that drug combination maximally suppressed tumor growth (FIG. 8D). Measurement of Pt in mouse tumors after 30 days of treatment with carboplatin and LB100/carboplatin showed a significant increase in intra-tumoral Pt levels upon combination treatment (FIG. 8E). IHC of the tumors confirmed that pMET, pp2A A, CD31 and Ki67 markers stained low in drug combination group (FIG. 9).

### Discussion/Conclusions

The present study demonstrates that LB100 alone or in combination with chemotherapeutic drugs inhibited cell proliferation and colony formation in SCLC. The maximum inhibitory effect on cell proliferation was observed with a combination of LB100 and carboplatin. Furthermore, the combination was effective in a spheroid model of SCLC that resembles the tumor microenvironment more closely. This drug combination also significantly inhibited invasion of the SCLC cells through HUVEC monolayer compared with the control untreated cells. These results, along with the fact that LB100/carboplatin combination was efficacious in significantly reducing tumor size and weight in a SCLC xenograft mouse model, underscore the potential of this innovative therapeutic option for SCLC.

In addition, LB100 treatment inhibited HGF-induced MET phosphorylation in SCLC cells. Consistent with our results, PP2A is known to regulate MET activation via dephosphorylation of S895 that leads to autophosphorylation of Y1234 and Y1235, resulting in activation of the receptor (34). Without wishing to be bound by theory, HGF-induced phosphorylation of MET appears to play an important role in epithelial-to-mesenchymal transition (EMT) in SCLC (22). In addition, the MET/HGF axis plays a major role in the development of chemoresistance in multiple tumor types, including lung cancer. In NSCLC, the activation of the MET receptor induced chemoresistance by inhibiting apoptosis via activation of PI3K-AKT pathway and downregulation of apoptosis-inducing factor (37). Blockade of this process with a MET inhibitor resensitized these cells to chemotherapy in vitro and in vivo (38). The fact that LB100 can subvert ligand activation of MET suggests that LB100 can also attenuate chemoresistance, a major impediment in treating SCLC. c-MET is also known to be involved in metabolic reprograming in several cancers (39-42).

Significant reduction of glucose uptake was observed, as well as glycolytic and OxPhos upon inhibiting PP2A activity with LB100 alone or in combination with carboplatin. Furthermore, the glycolytic capacity and oxidative capacity of these cells were reduced after these treatments. Without wishing to be bound by theory, these results suggest that the LB100 and carboplatin treatments lead to the reversal of the hybrid glycolysis/OxPhos phenotype, thus sensitizing the SCLC cells to the chemo drugs. Increased ATP production is associated with increased activity of the ATP-binding cassette (ABC) transporters resulting in chemoresistance (45) which is consistent with the fact that elevated ATP levels directly influence the activity of ABC transporters. Without wishing to be bound by theory, the inhibition of glycolysis, OxPhos and deprivation of ATP by LB100 may have led to attenuating the function of the efflux pump, thereby increasing the toxicity of the drug and reversing drug resistance.

Mass spectrometry data suggest that the Pt concentration in SCLC cells and tumor tissue was significantly increased after LB100 treatment. Copper influx/efflux transporters have been suggested to play an important role in platinum-based drug uptake and resistance (46) in cancer. A decrease in Copper transporter 1 (CTR1) expression and increase in ABC transporters, ATP 7A/7B efflux transporters, and multi-drug resistance protein MTB1 is observed in many cancers (47). Without wishing to be bound by theory, the observed increased uptake of Pt in SCLC could be due to the altered expression of one or more of the copper influx/efflux transporters in response to LB100. Consistent with this idea, a combination of LB100 and carboplatin acted synergistically to induce DNA damage and apoptosis in SCLC cells.

We have demonstrated that PD-L1 is overexpressed in neuroendocrine cells derived from a Rb^{f/f}/Trp53^{f/f} mouse model of SCLC (unpublished data) and combination of atezolizumab and LB100 in the presence of activated T cells induced the destruction of spheroids, led to infiltration of the activated T cells in the spheroids resulting in the dissociation of cells, loss of spheroid morphology and increased cell cytotoxicity.

Accordingly, the present data indicate that abrogation of PP2A with LB100 inhibits cell proliferation, tumor growth and metastasis by asserting its pleotropic effects on, the activity of the oncogene MET, energy production, and drug uptake via altering the expression of transporters thus increasing chemosensitivity. Furthermore, the present data also indicate that combining LB100 with carboplatin and etoposide can enhance these pleotropic effects of LB100 and that, combining immunotherapy with LB100 treatment led to increased T cells infiltration of H446 spheroids resulting in the disintegration of these spheroids. Taken together, the results from the present study suggest that pharmacologically targeting PP2A appears to be a viable strategy for SCLC.

### Materials and Methods

Tissue Microarray

Small cell lung cancer TMAs were from US Biomax Inc. (Rockville, Md.; LC818). Immunohistochemical (IHC) staining was performed using standard techniques previously described (49) with antibodies against PP2A A (CST, City of Industry, CA) in the Pathology/Solid tumor core, The City of Hope. Briefly, each TMA was reviewed and scored by two independent pathologists on a scale of 0 to 3: 0+, no staining, no expression; 1+, weak staining, low expression; 2+, moderate staining, moderate expression; and 3+, strong staining, high expression.

Cell Culture Reagents

Suspension SCLC H524, H526, H82, H446, H69 and H146 cells were purchased from ATCC (Manassas, Va.) and maintained in RPMI1640 (Corning Life Science, Tweksbury, Mass.) supplemented with 10% (v/v) fetal bovine serum and 1% (v/v) penicillin/streptomycin (Corning Life Science, Tweksbury, Mass.) and L-glutamine at 37° C. with 5% CO2. The morphology of the cell lines was monitored routinely, and the cell lines were routinely tested for mycoplasma with a mycoplasma detection kit (InvivoGen, San Diego, Calif.).

Immunoblotting

Whole cell lysates were prepared using RIPA lysis buffer and proteins were detected by immunoblotting using antibodies specific against PP2A A, PP2A C, Phospho-Histone H2AX (S139), MET, pMet (Tyr 1234/1235), Cleaved Caspase 3 and pan-Actin antibodies from CST (City of Industry, CA), Cleaved PARP1 (Santa Cruz Biotechnology, Dallas, Tex.) and pMET (Ser985) (ThermoFisher Scientific, Waltham, Mass.) were used as described previously (22).

Cell Viability Assay

To determine specific cytotoxicity, we used Cell Counting Kit-8 (Dojindo Molecular Technologies, Rockville, Md.) as previously described (50).

Colony Formation

Approximately, 1×103 cells in 0.3% agarose were seeded in a 96 well plate onto a layer of 0.6% agarose. Cells were grown in the present of LB100, carboplatin or LB100/carboplatin for three weeks to observe colony formation. The colonies were fixed in 4% formaldehyde and stained with crystal violet. Z-stacks of tiled bright field images were taken using a 5× objective with a step size of 200 microns on a Zeiss Observer 7 inverted microscope (Carl Zeiss, Obercohen, Germany). Using Zen Blue v2.5 (Carl Zeiss Microimaging), stacks were processed by first stitching a reference slice, and then the Extended Depth of Focus module, with default settings, was used to compress the Z-stack information into a single image. Manual counting was conducted on the resulting tiled image using the points tool, and summary measurements generated, in QuPath 0.1.3 (51).

PP2A Phosphatase Activity Measurement

PP2A immunoprecipitation Ser/Tre Phosphatase Assay Kit (Millipore, Temecula, Calif.) was used for measuring PP2A activity following manufacturer's protocol. Briefly, 8×106 H524 cells were treated with LB100 for 24 hours. The data are presented as the percentage of relative PP2A activity compared with control.

siPP2A subAα Transfection

Ser/Thr phosphatase 2A regulatory subunit A alpha isoform siRNA was purchased from MyBioSource (https://www.mybiosource.com/search/PPP2R1A-siRNA). Cells were transfected with 100 nM siRNA using jetPRIME reagent (Polyplus-transfection, LA, CA). siRNA transient transfection was verified with anti-PPP2R1A abs (MyBioSource, San Diego, Calif.).

Transendothelial Extravasation Assay

The ability of SCLC cells to invade though a layer of endothelial cells (ECs) was quantified using transendothelial monolayer resistance measurements using an electrical substrate-impedance sensing system (Applied Biophysics, Troy, N.Y.), as we have previously described (24).

Monitoring of Spheroid Growth and Cytotoxicity with the IncuCyte® Live-Cell Analysis System and IncuCyte® Cytotox Reagent

H446 cells were plated at a density of 10,000 cells per well and spheroid allowed to form (72-hours). Cells were then treated with LB100, Carboplatin or LB100/Carboplatin and kinetics of spheroid growth were obtained. Spheroids were imaged every 4 hours for 6 days and analyzed using the IncuCyte ZOOM software.

ICP-MS Assay

Samples were prepared and analyzed for Pt concentrations at the Isotoparium (California Institute of Technology), using precleaned Teflon beakers (PFA), Optima grade reagents (Fisher Chemical) and 18.2 MΩ Milli-Q water. Cell pellets were first digested in 500 μl of concentrated HNO3 for 30 minutes at 160° C., before complete dry down. Mouse tumors were digested in 1 mL of concentrated HNO3 for 30-45 minutes at 120° C. with periodic degassing, before complete dry down. Samples were cooled to room temperature, placed in 50:50 v/v concentrated HNO3:H2O2 (1 mL for cell pellets, 2 mL for tumors) in order to burn off organic matter. Cell pellets were placed on a hot plate overnight at 160° C. Tumors were heated at 120° C. for 8 hours with periodic degassing. All samples were then evaporated completely and reconstituted in 5 mL 3% v/v HNO3. Holmium (Spex Certiprep Assurance, Lot #24-80HOM) was used as the internal standard. A stock solution of 3% v/v HNO3 with 2 ppb Ho was used for all sample and standard dilutions. Aliquots of cell lines were diluted 20× using the HNO3+Ho stock solution, while tumor aliquots were diluted 100× using the same stock solution. Three technical replicates were measured per biological replicate to demonstrate reproducibility. All samples were analyzed using an iCAP RQ (ThermoFisher, Waltham, Mass.) ICP-MS and an SC-2 DX autosampler (Elemental Scientific, Omaha, Nebr.). Instrumental tuning parameters (e.g., nebulizer gas flow, torch alignment, and sample uptake rate, quadrupole ion deflector) were optimized to pass the standard performance check prior to analysis. A Pt standard curve (0.001, 0.01, 0.1, 1.0 ppb, Spex Certiprep Assurance, Lot #24-140PTM) was created using the HNO3 stock solution and measured for sample calibration. For each analysis, both Platinum 194 and 195 as well as Holmium 165 were measured. Each measurement used 5 main runs of 5 sweeps, and each sweep used a dwell time of 50 ms per isotope. To ensure that residual organics did not affect the concentration estimates, each sample was measured in two independent sessions (different days) using two different cone inserts (the High Matrix insert, typically used for geological samples, and the Robust insert, recommended for biological matrices). Both data sets are identical within uncertainty (≤±2%). Platinum mass was normalized to total protein mass for cell pellets and tumor mass for mouse samples.

Kinase Activity Profiling Using PamGene's Microarray Assay

H524 cells were treated with LB100 for 5 hours, to test the effects of the drug on protein tyrosine and serine/threonine kinase activity. PamChips were used to capture the activity of upstream kinases from either the tyrosine kinome (protein tyrosine kinase—PTK) or the serine/threonine kinome (serine/threonine kinase—STK). Both PamChips contain 144 peptides, each composed of 12-15 amino acids, with one or more phosphorylation sites. PTK and STK PamGene assays were performed according to the manufacturer's instructions. Samples were run in triplicate on the PamStation® 12 (PamGene, s-Hertogenbosch, Netherlands) by the High Throughput Screening Core (City of Hope, Duarte, Calif.). Image quantification and data processing were conducted with the Evolve and BioNavigator software package (PamGene). The peptides on each chip that had a significant (t test p<0.05) log fold change versus the untreated control for at least one drug concentration were analyzed using pathway enrichment analysis (http://reactome.org).

BiOLOG Metabolic Assay

Phenotype Microarrays (PMs) use a patented redox chemistry, employing cell respiration as a universal reporter. These assays potentially provide a natural fit to support data obtained from metabolomics screens. The redox assay provides for both amplification and precise quantitation of phenotypes. Redox dye mixes contain a water-soluble nontoxic tetrazolium reagent that can be used with virtually any type of animal cell line or primary cell (52). The dyes used in Biolog (Hayward, Calif., USA) assays measure output of nicotinamide adenine dinucleotide reduced form (NADH) production from various catabolic pathways present in the cells being tested. If cell growth is supported by the medium in an assay well, the actively metabolizing cells reduce the tetrazolium dye. Reduction of the dye results in colour formation in the well, and the phenotype is considered “positive.” If metabolism is hampered or growth is poor, then the phenotype is “weakly positive” or “negative,” and little or no color is formed in the well. This colorimetric redox assay allows examination of the effect of treatment on the metabolic rate produced by different substrates and thus is an excellent technique to combine with examination of metabolic output via metabolomics screens.

Glucose Uptake Assay

Glucose consumption was determined by using a colorimetric glucose assay (Invitrogen, Carlsbad, Calif.) following the manufacturer's instructions. Briefly, cells were seeded into 100 mm plates at a density 2×106 cells per well. After 48 hours of cell culture, supernatant of the medium was collected subjected into glucose detection. The uptake of glucose was determined compared with initial glucose concentration in the cell culture medium, which was taken as 100%.

Cell Energy Phenotype and Real Time ATP Rate

A Seahorse XF96 instrument (Agilent, Santa Clara, Calif.) was used for cell energy phenotype and real-time ATP assay. Cell energy phenotype assay measures mitochondrial respiration and glycolysis in basal and stressed levels. Real-time ATP measurement detects the rate of ATP production from glycolysis and mitochondria. Before experiment cells were treated for 18 hours with LB100. The day after being treated cells, were washed and seeded at a density 5×104 per well in 96 well plates treated with Cell-Tak. The plate was centrifuged to facilitate cell attachment and incubated at 37° C. for 60 min. Both assays were performed per manufacturer's instructions. Data analysis was done with Wave Desctop 2.6 software (Agilent, Santa Clara, Calif.).

Live Imaging of Spheroids with Drugs and T Cells

H446 were generated as described in Materials and Methods (Monitoring of spheroid growth and cytotoxicity with the IncuCyte® Live-Cell Analysis System and IncuCyte® Cytotox reagent) following incubation with T cells and drugs. The effect of LB100 and atezolizumab in the presence of T cells was monitored with IncuCyte 3D Multi-Tumor Spheroid assay.

Effect of LB100 on Tumor Growth in Subcutaneous H69 Cells Mouse Xenograft

Animal studies were performed according to an IACUC protocol approved by City of Hope National Medical Center Animal Care and Use Committee. Athymic nude mice (5-6 weeks of age) were purchased from NCI (Frederick, Md.). Mice were injected subcutaneously on their right flank with H69 cells suspended (2×106) in 100 μl of PBS and 100 μl of matrigel (BD Biosciences, San Jose, Calif.). Tumor growth was measured in two dimensions with caliper and when surface tumor was visible (45-50 mm2) mice were randomized in four groups as follow: vehicle (PBS, i.p. 3 times a week), LB100 (0.25 mg/kg, i.p. 3 times a week), carboplatin (50 mg/kg, i.p. 2 times a week) and drug combination (LB100/carboplatin i.p.) for 30 days. At the end of the study, the mice were euthanized by CO2 asphyxiation followed by cervical dislocation. Tumor tissues were excised, weighed, and subsequently fixed in 10% buffered formalin and embedded in paraffin for histological analysis.

Statistical Analysis

Statistical analyses were conducted using GraphPad Prism 8. Two sample groups were compared by unpaired, two-sided Student's t tests. Data of more than two groups were analyzed by one-way ANOVA followed by Tukey's multiple comparison tests. Values of p<0.05 were considered significant and indicated as: *p<0.05, **p<0.01, ***p<0.001. Graphs represent the mean±standard error of the mean. (SE)

### References (Example 1)

1. S. Reynhout, V. Janssens, Physiologic functions of PP2A: Lessons from genetically modified mice. Biochim Biophys Acta Mol Cell Res 1866, 31-50 (2019).
2. S. Mazhar, S. E. Taylor, J. Sangodkar, G. Narla, Targeting PP2A in cancer: Combination therapies. Biochim Biophys Acta Mol Cell Res 1866, 51-63 (2019).
3. J. J. Thompson, C. S. Williams, Protein Phosphatase 2A in the Regulation of Wnt Signaling, Stem Cells, and Cancer. Genes (Basel) 9, (2018).
4. O. Kauko, J. Westermarck, Non-genomic mechanisms of protein phosphatase 2A (PP2A) regulation in cancer. Int J Biochem Cell Biol 96, 157-164 (2018).
5. G. Xiao, L. N. Chan, L. Klemm, D. Braas, Z. Chen, H. Geng, Q. C. Zhang, A. Aghajanirefah, K. N. Cosgun, T. Sadras, J. Lee, T. Mirzapoiazova, R. Salgia, T. Ernst, A. Hochhaus, H. Jumaa, X. Jiang, D. M. Weinstock, T. G. Graeber, M. Mischen, B-Cell-Specific Diversion of Glucose Carbon Utilization Reveals a Unique Vulnerability in B Cell Malignancies. Cell 173, 470-484 e418 (2018).
6. J. Lu, J. S. Kovach, F. Johnson, J. Chiang, R. Hodes, R. Lonser, Z. Zhuang, Inhibition of serine/threonine phosphatase PP2A enhances cancer chemotherapy by blocking DNA damage induced defense mechanisms. Proc Natl Acad Sci USA 106, 11697-11702 (2009).
7. J. S. Kovach, F. Johnson, U. States, Ed. (Lixte Biotechnology Inc, United States, 2011).
8. X. Bai, X. Zhi, Q. Zhang, F. Liang, W. Chen, C. Liang, Q. Hu, X. Sun, Z. Zhuang, T. Liang, Inhibition of protein phosphatase 2A sensitizes pancreatic cancer to chemotherapy by increasing drug perfusion via HIF-1α-VEGF mediated angiogenesis. Cancer Lett 355, 281-287 (2014).
9. C. Hu, M. Yu, Y. Ren, K. Li, D. M. Maggio, C. Mei, L. Ye, J. Wei, J. Jin, Z. Zhuang, H. Tong, PP2A inhibition from LB100 therapy enhances daunorubicin cytotoxicity in secondary acute myeloid leukemia via miR-181b-1 upregulation. Sci Rep 7, 2894 (2017).
10. C. Zhang, Y. Peng, F. Wang, X. Tan, N. Liu, S. Fan, D. Wang, L. Zhang, D. Liu, T. Wang, S. Wang, Y. Zhou, Y. Su, T. Cheng, Z. Zhuang, C. Shi, A synthetic cantharidin analog for the enhancement of doxorubicin suppression of stem cell-derived aggressive sarcoma. Biomaterials 31, 9535-9543 (2010).
11. L. Martiniova, J. Lu, J. Chiang, M. Bernardo, R. Lonser, Z. Zhuang, K. Pacak, Pharmacologic modulation of serine/threonine phosphorylation highly sensitizes PHEO in a MPC cell and mouse model to conventional chemotherapy. PLoS One 6, e14678 (2011).
12. D. Wei, L. A. Parsels, D. Karnak, M. A. Davis, J. D. Parsels, A. C. Marsh, L. Zhao, J. Maybaum, T. S. Lawrence, Y. Sun, M. A. Morgan, Inhibition of protein phosphatase 2A radiosensitizes pancreatic cancers by modulating CDC25C/CDK1 and homologous recombination repair. Clin Cancer Res 19, 4422-4432 (2013).
13. P. Lv, Y. Wang, J. Ma, Z. Wang, J. L. Li, C. S. Hong, Z. Zhuang, Y. X. Zeng, Inhibition of protein phosphatase 2A with a small molecule LB100 radiosensitizes nasopharyngeal carcinoma xenografts by inducing mitotic catastrophe and blocking DNA damage repair. Oncotarget 5, 7512-7524 (2014).
14. I. K. Gordon, J. Lu, C. A. Graves, K. Huntoon, J. M. Frerich, R. H. Hanson, X. Wang, C. S. Hong, W. Ho, M. J. Feldman, B. Ikejiri, K. Bisht, X. S. Chen, A. Tandle, C. Yang, W. T. Arscott, D. Ye, J. D. Heiss, R. R. Lonser, K. Camphausen, Z. Zhuang, Protein Phosphatase 2A Inhibition with LB100 Enhances Radiation-Induced Mitotic Catastrophe and Tumor Growth Delay in Glioblastoma. Mol Cancer Ther 14, 1540-1547 (2015).
15. K. E. Chang, B. R. Wei, J. P. Madigan, M. D. Hall, R. M. Simpson, Z. Zhuang, M. M. Gottesman, The protein phosphatase 2A inhibitor LB100 sensitizes ovarian carcinoma cells to cisplatin-mediated cytotoxicity. Mol Cancer Ther 14, 90-100 (2015).
16. W. S. Ho, M. J. Feldman, D. Maric, L. Amable, M. D. Hall, G. M. Feldman, A. Ray-Chaudhury, M. J. Lizak, J. C. Vera, R. A. Robison, Z. Zhuang, J. D. Heiss, PP2A inhibition with LB100 enhances cisplatin cytotoxicity and overcomes cisplatin resistance in medulloblastoma cells. Oncotarget 7, 12447-12463 (2016).
17. W. S. Ho, H. Wang, D. Maggio, J. S. Kovach, Q. Zhang, Q. Song, F. M. Marincola, J. D. Heiss, M. R. Gilbert, R. Lu, Z. Zhuang, Pharmacologic inhibition of protein phosphatase-2A achieves durable immune-mediated antitumor activity when combined with PD-1 blockade. Nat Commun 9, 2126 (2018).
18. R. V. Parry, J. M. Chemnitz, K. A. Frauwirth, A. R. Lanfranco, I. Braunstein, S. V. Kobayashi, P. S. Linsley, C. B. Thompson, J. L. Riley, CTLA-4 and PD-1 receptors inhibit T-cell activation by distinct mechanisms. Mol Cell Biol 25, 9543-9553 (2005).
19. P. Zhou, D. R. Shaffer, D. A. Alvarez Arias, Y. Nakazaki, W. Pos, A. J. Torres, V. Cremasco, S. K. Dougan, G. S. Cowley, K. Elpek, J. Brogdon, J. Lamb, S. J. Turley, H. L. Ploegh, D. E. Root, J. C. Love, G. Dranoff, N. Hacohen, H. Cantor, K. W. Wucherpfennig, In vivo discovery of immunotherapy targets in the tumour microenvironment. Nature 506, 52-57 (2014).
20. I. Kawada, R. Hasina, F. E. Lennon, V. P. Bindokas, P. Usatyuk, Y. H. Tan, S. Krishnaswamy, Q. Arif, G. Carey, R. D. Hseu, M. Robinson, M. Tretiakova, T. M. Brand, M. Iida, M. K. Ferguson, D. L. Wheeler, A. N. Husain, V. Natarajan, E. E. Vokes, P. A. Singleton, R. Salgia, Paxillin mutations affect focal adhesions and lead to altered mitochondrial dynamics: relevance to lung cancer. Cancer Biol Ther 14, 679-691 (2013).
21. L. Faoro, P. A. Singleton, G. M. Cervantes, F. E. Lennon, N. W. Choong, R. Kanteti, B. D. Ferguson, A. N. Husain, M. S. Tretiakova, N. Ramnath, E. E. Vokes, R. Salgia, EphA2 mutation in lung squamous cell carcinoma promotes increased cell survival, cell invasion, focal adhesions, and mammalian target of rapamycin activation. J Biol Chem 285, 18575-18585 (2010).
22. R. Kanteti, I. Dhanasingh, I. Kawada, F. E. Lennon, Q. Arif, R. Bueno, R. Hasina, A. N. Husain, W. Vigneswaran, T. Seiwert, H. L. Kindler, R. Salgia, MET and PI3K/mTOR as a potential combinatorial therapeutic target in malignant pleural mesothelioma. PLoS One 9, e105919 (2014).
23. E. C. Finger, A. J. Giaccia, Hypoxia, inflammation, and the tumor microenvironment in metastatic disease. Cancer Metastasis Rev 29, 285-293 (2010).
24. T. Mirzapoiazova, N. Mambetsariev, F. E. Lennon, B. Mambetsariev, J. E. Berlind, R. Salgia, P. A. Singleton, HABP2 is a Novel Regulator of Hyaluronan-Mediated Human Lung Cancer Progression. Front Oncol 5, 164 (2015).
25. Y. Wang, R. Yang, J. Gu, X. Yin, N. Jin, S. Xie, Y. Wang, H. Chang, W. Qian, J. Shi, K. Iqbal, C. X. Gong, C. Cheng, F. Liu, Cross talk between PI3K-AKT-GSK-3p and PP2A pathways determines tau hyperphosphorylation. Neurobiol Aging 36, 188-200 (2015).
26. G. P. Liu, W. Wei, X. Zhou, H. R. Shi, X. H. Liu, G. S. Chai, X. Q. Yao, J. Y. Zhang, C. X. Peng, J. Hu, X. C. Li, Q. Wang, J. Z. Wang, Silencing PP2A inhibitor by lenti-shRNA interference ameliorates neuropathologies and memory deficits in tg2576 mice. Mol Ther 21, 2247-2257 (2013).
27. D. Perrotti, P. Neviani, Protein phosphatase 2A: a target for anticancer therapy. Lancet Oncol 14, e229-238 (2013).
28. F. H. Duong, M. T. Dill, M. S. Matter, Z. Makowska, D. Calabrese, T. Dietsche, S. Ketterer, L. Terracciano, M. H. Heim, Protein phosphatase 2A promotes hepatocellular carcinogenesis in the diethylnitrosamine mouse model through inhibition of p53. Carcinogenesis 35, 114-122 (2014).
29. F. H. Duong, M. Filipowicz, M. Tripodi, N. La Monica, M. H. Heim, Hepatitis C virus inhibits interferon signaling through up-regulation of protein phosphatase 2A. Gastroenterology 126, 263-277 (2004).
30. L. Liu, H. Wang, J. Cui, Q. Zhang, W. Zhang, W. Xu, H. Lu, S. Liu, S. Shen, F. Fang, L. Li, W. Yang, Z. Zhuang, J. Li, Inhibition of Protein Phosphatase 2A Sensitizes Mucoepidermoid Carcinoma to Chemotherapy via the PI3K-AKT Pathway in Response to Insulin Stimulus. Cell Physiol Biochem 50, 317-331 (2018).
31. R. Salgia, Role of c-Met in cancer: emphasis on lung cancer. Semin Oncol 36, S52-58 (2009).
32. M. Hardy-Werbin, R. Del Rey-Vergara, M. A. Galindo-Campos, L. Moliner, E. Arriola, MET Inhibitors in Small Cell Lung Cancer: From the Bench to the Bedside. Cancers (Basel) 11, (2019).
33. L. Gandino, P. Longati, E. Medico, M. Prat, P. M. Comoglio, Phosphorylation of serine 985 negatively regulates the hepatocyte growth factor receptor kinase. J Biol Chem 269, 1815-1820 (1994).
34. A. Hashigasako, M. Machide, T. Nakamura, K. Matsumoto, T. Nakamura, Bi-directional regulation of Ser-985 phosphorylation of c-met via protein kinase C and protein phosphatase 2A involves c-Met activation and cellular responsiveness to hepatocyte growth factor. J Biol Chem 279, 26445-26452 (2004).
35. A. R. Virzi, A. Gentile, S. Benvenuti, P. M. Comoglio, Reviving oncogenic addiction to MET bypassed by BRAF (G469A) mutation. Proc Natl Acad Sci USA 115, 10058-10063 (2018).
36. V. Chung, A. S. Mansfield, F. Braiteh, D. Richards, H. Durivage, R. S. Ungerleider, F. Johnson, J. S. Kovach, Safety, Tolerability, and Preliminary Activity of LB-100, an Inhibitor of Protein Phosphatase 2A, in Patients with Relapsed Solid Tumors: An Open-Label, Dose Escalation, First-in-Human, Phase I Trial. Clin Cancer Res 23, 3277-3284 (2017).
37. J. T. Chen, C. Y. Huang, Y. Y. Chiang, W. H. Chen, S. H. Chiou, C. Y. Chen, K. C. Chow, HGF increases cisplatin resistance via down-regulation of AIF in lung cancer cells. Am J Respir Cell Mol Biol 38, 559-565 (2008).
38. I. Cañadas, F. Rojo, Á. Taus, O. Arpi, M. Arumi-Uria, L. Pijuan, S. Menéndez, S. Zazo, M. Dómine, M. Salido, S. Mojal, A. Garcia de Herreros, A. Rovira, J. Albanell, E. Arriola, Targeting epithelial-to-mesenchymal transition with Met inhibitors reverts chemoresistance in small cell lung cancer. Clin Cancer Res 20, 938-950 (2014).
39. V. Boschert, N. Klenk, A. Abt, S. Janaki Raman, M. Fischer, R. C. Brands, A. Seher, C. Linz, U. D. A. Muller-Richter, T. Bischler, S. Hartmann, The Influence of Met Receptor Level on HGF-Induced Glycolytic Reprogramming in Head and Neck Squamous Cell Carcinoma. Int J Mol Sci 21, (2020).
40. T. Thi Thu Nguyen, E. Shang, G. Karpel-Massler, M. D. Siegelin, Metabolic Reprogramming by c-MET Inhibition as a Targetable Vulnerability in Glioblastoma. Oncoscience 7, 14-16 (2020).
41. F. Meng, L. Wu, L. Dong, A. V. Mitchell, C. James Block, J. Liu, H. Zhang, Q. Lu, W. M. Song, B. Zhang, W. Chen, J. Hu, J. Wang, Q. Yang, M. Huttemann, G. Wu, EGFL9 promotes breast cancer metastasis by inducing cMET activation and metabolic reprogramming. Nat Commun 10, 5033 (2019).
42. N. Jin, A. Bi, X. Lan, J. Xu, X. Wang, Y. Liu, T. Wang, S. Tang, H. Zeng, Z. Chen, M. Tan, J. Ai, H. Xie, T. Zhang, D. Liu, R. Huang, Y. Song, E. L. Leung, X. Yao, J. Ding, M. Geng, S. H. Lin, M. Huang, Identification of metabolic vulnerabilities of receptor tyrosine kinases-driven cancer. Nat Commun 10, 2701 (2019).
43. L. Yu, M. Lu, D. Jia, J. Ma, E. Ben-Jacob, H. Levine, B. A. Kaipparettu, J. N. Onuchic, Modeling the Genetic Regulation of Cancer Metabolism: Interplay between Glycolysis and Oxidative Phosphorylation. Cancer Res 77, 1564-1574 (2017).
44. D. Jia, J. H. Park, K. H. Jung, H. Levine, B. A. Kaipparettu, Elucidating the Metabolic Plasticity of Cancer: Mitochondrial Reprogramming and Hybrid Metabolic States. Cells 7, (2018).
45. L. Ma, X. Zong, Metabolic Symbiosis in Chemoresistance: Refocusing the Role of Aerobic Glycolysis. Front Oncol 10, 5 (2020).
46. D. Kilari, E. Guancial, E. S. Kim, Role of copper transporters in platinum resistance. World J Clin Oncol 7, 106-113 (2016).
47. C. A. Rabik, M. E. Dolan, Molecular mechanisms of resistance and toxicity associated with platinating agents. Cancer Treat Rev 33, 9-23 (2007).
48. G. L. Coles, S. Cristea, J. T. Webber, R. S. Levin, S. M. Moss, A. He, J. Sangodkar, Y. C. Hwang, J. Arand, A. P. Dramas, N. A. Mooney, J. Demeter, J. N. Spradlin, B. Mauch, V. Le, Y. T. Shue, J. H. Ko, M. C. Lee, C. Kong, D. K. Nomura, M. Ohlmeyer, D. L. Swaney, N. J. Krogan, P. K. Jackson, G. Narla, J. D. Gordan, K. M. Shokat, J. Sage, Unbiased Proteomic Profiling Uncovers a Targetable GNAS/PKA/PP2A Axis in Small Cell Lung Cancer Stem Cells. Cancer Cell 38, 129-143 e127 (2020).
49. P. C. Ma, M. S. Tretiakova, A. C. MacKinnon, N. Ramnath, C. Johnson, S. Dietrich, T. Seiwert, J. G. Christensen, R. Jagadeeswaran, T. Krausz, E. E. Vokes, A. N. Husain, R. Salgia, Expression and mutational analysis of MET in human solid cancers. Genes Chromosomes Cancer 47, 1025-1037 (2008).
50. J. Wang, T. Mirzapoiazova, Y. H. Carol Tan, K. M. Pang, A. Pozhitkov, Y. Wang, Y. Wang, B. Mambetsariev, E. Wang, M. W. Nasser, S. K. Batra, D. Raz, K. Reckamp, P. Kulkari, Y. Zheng, R. Salgia, Inhibiting crosstalk between MET signaling and mitochondrial dynamics and morphology: a novel therapeutic approach for lung cancer and mesothelioma. Cancer Biol Ther 19, 1023-1032 (2018).
51. P. Bankhead, M. B. Loughrey, J. A. Femindez, Y. Dombrowski, D. G. McArt, P. D. Dunne, S. McQuaid, R. T. Gray, L. J. Murray, H. G. Coleman, J. A. James, M. Salto-Tellez, P. W. Hamilton, QuPath: Open source software for digital pathology image analysis. Sci Rep 7, 16878 (2017).
52. B. R. Bochner, M. Siri, R. H. Huang, S. Noble, X. H. Lei, P. A. Clemons, B. K. Wagner, Assay of the multiple energy-producing pathways of mammalian cells. PLoS One 6, e18147 (2011).

### Example 2. A Phase Ib Open-Label Study of LB-100 in Combination with Carboplatin/Etoposide/Atezolizumab in Untreated Extensive-Stage Small Cell Lung Carcinoma

Study Rationale: More than one million people died from lung cancer worldwide in 2017, and small cell carcinomas account for approximately 15% of all lung cancers. Even with double or triple drug therapy combinations, median survival for SCLC with “extensive disease” (ED-SCLC, 70% of patients) is only approximately 9 months and overall 5-year survival remains at around 5%. PP2A is ubiquitously expressed in SCLC cells (unpublished data), however, its potential relevance in SCLC remains mostly unknown. Protein phosphatase 2A (PP2A) is a phosphatase involved in the regulation of key oncoproteins, such as c-Myc and Bcr-Abl in a wide range of cancer subtypes including lung cancers and B cell-derived leukemias. LB-100 is a potent and selective antagonist of PP2A that has shown efficacy in a number of pre-clinical models. The combination of LB-100 with carboplatin, etoposide and atezolizumab, the standard of care for ED-SCLC, will be evaluated in treatment naïve patients to determine the recommended phase II dose (RP2D).

Goals: This is a Phase Ib open label study for subjects with extensive-stage disease SCLC who have not received prior treatment with systemic therapy for SCLC. The Phase Ib study is a single arm study expected to enroll 18 evaluable patients (maximum 30) entered in groups of 3 at escalating doses of LB-100 using the traditional 3+3 design. Patients will receive induction therapy with carboplatin/etoposide/atezolizumab for 4 cycles. Each cycle is defined as 3 weeks (21 days). Patients will then proceed to maintenance with LB-100 and atezolizumab. Patients who discontinue study therapy without disease progression will continue to be evaluated for tumor response using RECIST v1.1 (Appendix B) guidelines every 6-8 weeks until disease progression, death, or study closure. The primary endpoint is to determine the recommended phase II dose (RP2D) of LB-100 plus carboplatin/etoposide/atezolizumab in patients with extensive-stage small cell lung carcinoma.

Objectives: The primary objective of this study is to determine the recommended Phase II dose (RP2D) of LB-100 when given in combination with standard doses of carboplatin, etoposide and atezolizumab in treatment naïve patients with extensive-stage small cell lung cancer (ED-SCLC).

The secondary objectives of the study are:
Progression Free Survival (PFS)
Objective response rate (ORR)
Overall survival (OS)
Duration of overall response (DOR)
Safety/Adverse events

Exploratory objectives of the study are:
The pharmacokinetics (PK) of LB-100 and etoposide
The biomarkers relevant to LB-100 and the disease state as well as their correlation to clinical outcomes

Study Design:

Dose Escalation: The Phase I dose-finding will use a traditional 3+3 to determine the maximum tolerated dose (MTD), based on first cycle DLTs. A maximum of 4 dose levels of LB-100 will be explored. The determination of the recommended Phase II dose (RP2D) will be based on the MTD (and will not exceed the MTD) with additional consideration of dose modifications, adverse events in subsequent cycles, clinical activity and correlative studies.

Expanded Cohort: Additional patients will be enrolled until 12 patients are treated at the proposed RP2D to help confirm the tolerability of the RP2D and obtain preliminary data on efficacy.

Primary and Secondary Endpoints:

Primary Endpoints:
Determine recommended phase II dose (RP2D) of the combination using DLT during the first cycle as assessed by CTCAE version 5.0

Secondary Endpoints:
Objective response rate (ORR) by RECIST v1.1
Duration of overall response by RECIST v1.1
Safety and Adverse events by assessed by CTCAE version 5.0
Progression-free survival (PFS) as defined by RECIST v1.1
Overall survival, which is defined as the time from the date of study enrollment to the date of death from any cause. For patients who are still alive as of the data cutoff date, OS time will be censored on the date of the patient's last contact (last contact for patients in post discontinuation is last known alive date in mortality status).

Sample Size Accrual Study Duration:

Sample Size: Minimum=14, Maximum=30, Expected=18

Estimated Accrual Duration: 1-1.5 years

Estimated Study Duration: 18-24 months

Estimated Participant Duration: 6 months

Abbreviated Eligibility Criteria:

Main Inclusion Criteria:
Histologically or cytologically confirmed extensive-stage disease small cell lung carcinoma per the Veterans Administration Lung Study Group (VALG) staging system
Measurable disease as defined by the Response Evaluation Criteria in Solid Tumors (RECIST)
No prior systemic chemotherapy, immunotherapy, biological, hormonal, or investigational therapy for SCLC
Adequate hematologic and organ function, including:
Hematologic: absolute neutrophil (segmented and bands) count (ANC) ≥1.5×10/L,
platelets ≥100×10/L, and hemoglobin ≥9 g/dL
Hepatic: bilirubin ≤1.5 times upper limits of normal (ULN) may be enrolled, and alkaline phosphatase (AP), alanine aminotransferase (ALT) and aspartate aminotransferase (AST)≤3.0 times ULN (AP, AST, and ALT ≤5 times ULN are acceptable if the liver has tumor involvement).
Renal: calculated creatinine clearance (CrCl) ≥60 mL/min based on the Cockcroft and Gault formula
at least 18 years old at the time of screening
estimated life expectancy of at least 12 weeks

Main Exclusion Criteria:
currently enrolled in, or discontinued within the last 30 days from, a clinical trial involving an investigational product or non-approved use of a drug or device, or concurrently enrolled in any other type of medical research judged not to be scientifically or medically compatible with this study
Diagnosis of NSCLC or mixed NSCLC and SCLC
No prior malignancy other than SCLC, carcinoma in situ of the cervix, or nonmelanoma skin cancer, unless that prior malignancy was diagnosed and definitively treated 5 or more years prior to study entry with no subsequent evidence of recurrence. Patients with a history of low grade (Gleason score ≤6=Grade Group 1) localized prostate cancer will be eligible even if diagnosed less than 5 years prior to study entry
serious concomitant systemic disorder that, in the opinion of the investigator, would compromise the patient's ability to adhere to the protocol
active or ongoing infection during screening requiring the use of systemic antibiotics
serious cardiac condition, such as myocardial infarction within 6 months, angina, or heart disease as defined by the New York Heart Association Class III or IV
clinical evidence of central nervous system (CNS) metastases or leptomeningeal carcinomatosis, except for individuals who have previously-treated CNS metastases, are asymptomatic, and have had no requirement for steroid medication for 1 week prior to the first dose of study drug and have completed radiation 2 weeks prior to the first dose of study drug
known or suspected allergy to any agent given in association with this trial
pregnant or lactating women
History of autoimmune disease, including minor/mild autoimmune disease not requiring immunosuppressants (such as eczema on less than 10% of the body surface area and long term diabetes mellitus type Ion stable insulin).
Known hepatitis B or hepatitis C
Known human immunodeficiency virus (HIV) positive
Treatment with systemic corticosteroid or other immunosuppressive medication. The use of inhaled corticosteroids for chronic obstructive pulmonary disease, mineralocorticoids (e.g., fludrocortisone) for patients with orthostatic hypotension, and low-dose supplemental corticosteroids for adrenocortical insufficiency are allowed.
Administration of a live, attenuated vaccine within 28 days prior to study
Uncontrolled pleural effusion, pericardial effusion, or ascites requiring recurrent drainage procedures (once monthly or more frequently). Patients with indwelling catheters are allowed.
Uncontrolled or symptomatic hypercalcemia (>1.5 mmol/L ionized calcium or calcium >12 mg/dL or corrected serum calcium >ULN). Patients who are receiving denosumab prior to study entry must be willing and eligible to discontinue its use and replace it with a bisphosphonate while in the study.
History of idiopathic pulmonary fibrosis, organizing pneumonia (e.g., bronchiolitis obliterans), drug-induced pneumonitis, idiopathic pneumonitis, or evidence of active pneumonitis on screening chest CT scan. History of radiation pneumonitis in the radiation field (fibrosis) is permitted.
Prior allogeneic bone marrow transplantation or solid organ transplant.
QTcF (Fridericia Correction Formula) >470 on 2 out of 3 EKG's.
Diagnosis of congenital long QT syndrome
Treatment, within 7 days prior to first dose of study drug, with medications that are known to prolong the QT interval and/or are associated with a risk of Torsades de Pointes.
Treatment with CYP450 substrates within 7 days prior to first dose of study drug.
Treatment with nephrotoxic compounds within 7 days prior to first dose of study drug.
Treatment with warfarin within 7 days prior to first dose of study drug.
Treatment with antiepileptic medications that may increase etoposide clearance (including but not limited to phenytoin, phenobarbital, carbamazepine, and valproic acid) within 7 days prior to first dose of study drug.
Treatment with strong P-glycoprotein inhibitors within 7 days prior to first dose of study drug.
Subjects, who in the opinion of the investigator, may not be able to comply with the safety monitoring requirements of the study.

Investigational Product Dosage and Administration

One Cycle is 21 Days. Patients will receive 4 cycles of induction LB-100+atezolizumab/carboplatin/etoposide and then will proceed to maintenance with atezolizumab+LB-100.

LB-100: Intravenous (IV) at assigned dose (0.83, 1.25, 1.75, 2.33 or 3.10 mg/m2), over 15 minutes, given first, Days 1 & 3 of each cycle during induction and maintenance. Other drugs should be given 1 hour after the end of the LB-100 infusion.

Atezolizumab: 1,200 mg IV after LB-100, Day 1 of each cycle during induction and maintenance. Infused over 60 (+15) minutes (for first infusion, shortening to 30 [+10] minutes for subsequent infusions, depending on patient tolerance), given after LB-100.

Carboplatin: 5 AUC IV, after the atezolizumab, over 30-60 minutes, Day 1 of each cycle during induction.

Etoposide: 100 mg/m2 IV, given last (after the carboplatin on Day 1 of each cycle, by itself. Day 2 of each cycle, after LB-100 Day 3 of each cycle) during induction. Infused over 60 minutes.

Treatment Overview: This Phase Ib study of LB-100 diluted in 50 mL of normal saline for injection will be administered intravenously in the outpatient clinic over 15 minutes in patients with extensive-stage small cell lung cancer. Patients will receive an intravenous infusion of LB-100 diluted in 50 mL of normal saline (0.9%) over 15+/−5 minutes on days 1 and 3 of each 21 day cycle at escalating doses starting at Dose Level 1 (see Table 5.1). The LB-100 should be given first and should end one hour before the start of other drugs. All three patients at each dose level will be assessed for evidence of limiting toxicity through their return visit day 21 (and any delay prior to the start of cycle 2) before the decision is made for dose escalation in the next cohort. The MTD is defined as the highest dose level below which DLT is manifested in ≥33% of the patients (unless the highest dose to be tested does not have ≥33% of patients with a DLT) and where at least 6 patients have been treated.

The study is based on a standard 3+3 patient dose escalation design. It is planned that there will be 3 possible dose escalations (and one possible de-escalation level if needed). Thus, a maximum of 24 patients will be enrolled during dose finding, with an expected sample-size of 12 during escalation/de-escalation (additional patients to achieve 12 patients at the RP2D will follow for an expected sample-size of 18 total patients and maximum of 30).

All patients who are not evaluable for DLT (dose-limiting toxicity) will be replaced. Patients who do not receive the planned doses without a DLT, will be considered inevaluable as will patients where inadequate follow-up assessments are conducted for reasons unrelated to toxicity. Patients will be enrolled at most in cohorts of 3. If 0/3 patients have a DLT attributable to the combination, then the next 3 patients will be treated at the next dose level. If a DLT treatment occurs in 1/3 patients, then 3 more patients (for a total of 6) will be treated at the same dose level. If no additional DLT attributable to treatment is observed at the expanded dose level (i.e. 1/6 with DLT), then the LB-100 dose will be escalated to the next level. If two or more patients (i.e. 2/6) have a DLT then one level below that dose will be tested.

Dose escalation will terminate as soon as two or more patients have a DLT at a given dose level or the highest dose level is tested. There will be no dose escalation within a patient.

The MTD is defined as the highest LB-100 dose tested in which none or only one patient had a DLT during the first cycle of therapy, when at least six patients were treated at that dose and are evaluable for toxicity assessment. The MTD is one dose level below the lowest dose tested in which 2 patients had a DLT attributable to treatment unless the highest dose is deemed safe. In addition to these rules, all dose modifications and later cycle toxicities will be reviewed prior to escalation or expansion and can modify the decision to be more conservative (e.g. to not escalate when the standard rules state escalate, or de-escalate when the standard rules state expand the dose).

Any severe immune-related event that requires discontinuation of therapy will also prompt a review by the DSMC, regardless of cycle of therapy.

Dose Levels: LB-100 on Days 1 and 3 of a 21 Day cycle, at escalating doses prior to standard doses of carboplatin/atezolizumab/etoposide

TABLE 1

| Dose Level | LB-100 (mg/m²) |
|---|---|
| −1^{(a)} | 0.83 |
| 1 (Starting dose) | 1.25 |
| 2 | 1.75 |
| 3 | 2.33 |
| 4 | 3.10 |

^{(a)}In the event that 2 or more DLT's are observed at Dose Level 1, subsequent patients will be enrolled in Dose Level −1.

LB-100: LB-100 is supplied as a sterile solution for intravenous administration. LB-100 is stored at −20 SC (range: −25° C. to −10° C.). Each vial contains 10 mL of LB-100 at a concentration of 1 mg/mL. The proper dose is drawn up in a sterile syringe and added to 50 mL of normal saline (0.9%) and infused over 15+/−5 minutes prior to administration of atezolizumab on Day 1 and prior to etoposide on Day 3. Following dilution in normal saline, LB-100 should be administered within 4 hours.

Carboplatin: Carboplatin is supplied as a sterile lyophilized powder available in single-dose vials containing 50 mg, 150 mg and 450 mg of carboplatin for administration by intravenous injection. Each vial contains equal parts by weight of carboplatin and mannitol. Immediately before use, the content of each vial must be reconstituted with either Sterile Water for Injection, USP, 5% Dextrose in Water, or 0.9% Sodium Chloride Injection, USP, according to the following schedule (Table 2):

TABLE 2

| Vial Strength | Diluent Volume |
|---|---|
| 50 mg | 5 mL |
| 150 mg | 15 mL |
| 450 mg | 45 mL |

These dilutions all produce a carboplatin concentration of 10 mg/mL. Carboplatin can be further diluted to concentrations as low as 0.5 mg/mL with 5% Dextrose in Water or 0.9% Sodium Chloride Injection, USP (NS).

VP-16 (Etoposide): 100 mg of VP-16 is supplied as 5 mL of solution in Sterile Multiple Dose Vials for injection. The pH of the yellow clear solution is 3-4. Each mL contains 20 mg VP-16, 2 mg citric acid, 30 mg benzyl alcohol, 80 mg polysorbate 80/tween 80, 650 mg polyethylene glycol 300 and 30.5% (v/v) alcohol. VP-16 must be diluted prior to use with either 5% Dextrose Injection, USP or 0.95 sodium Chloride Injection, USP. The time before precipitation occurs depends on concentration, however, when at a concentration of 0.2 mg/mL it is stable for 96 hours at room temperature and at 0.4 mg/mL it is stable for 48 hours.

Atezolizumab (Tecentriq): Atezolizumab is a sterile, preservative-free, and colorless to slightly yellow solution for intravenous infusion supplied as a carton containing one 1200 mg/20 mL single-dose vial (NDC 50242-917-01). Store vials under refrigeration at 2° C. to 8° C. (36° F. to 46° F.) in original carton to protect from light. Do not freeze. Do not shake.

Study drug schedule, dose, route and timing: The induction phase is four cycles (Cycles 1-4). The maintenance phase is Cycle 5 and beyond.

TABLE 3

| Drug | Dose | Route | Schedule | Notes |
|---|---|---|---|---|
| LB-100 | As assigned (.83, | IV | Days 1 and 3 of each | Infused over 15 |
| (Induction and | 1.25, 1.75, 2.33 or | | 21 day cycle during | minutes. Given first. |
| Maintenance) | 3.10 mg/m²) | | the induction phase | Other drugs should |
| | | | (Cycles 1-4) and | start 1 hour after end of |
| | | | maintenance phase | LB-100 infusion. |
| | | | (Cycle 5 onward) | |
| Atezolizumab | 1200 mg/20 mL | IV | Day 1 of each 21 | Infused over 60 (±15) |
| (Tecentriq) | | | day cycle during the | minutes (for first |
| (Induction and | | | induction phase | infusion, shortening to |
| Maintenance) | | | (Cycles 1-4) and | 30 [±10] minutes for |
| | | | maintenance phase | subsequent infusions, |
| | | | (Cycle 5 onward) | depending on patient |
| | | | | tolerance. |
| Carboplatin | AUC 5 | IV | Day 1 of the 21 day | Given after |
| (Induction) | | | cycle; repeat every | atezolilzumab. Infused |
| | | | 21 days for 4 cycles | over 30-60 minutes. |
| VP-16 | 100 mg/m² | IV | Days 1, 2 and 3 of | Given last. Infused over |
| (Etoposide) | | | the 21 day cycle; | 60 minutes. |
| (Induction) | | | repeat every 21 days | |
| | | | for 4 cycles | |

Planned Duration of Therapy: Within 4 weeks before the first dose of study treatment, baseline tumor measurement(s) will be performed on each patient. At baseline: computed tomography (CT) [or magnetic resonance imaging (MRI)] of the head, chest, abdomen, pelvis, and a bone and/or PET scan. Ultrasound will not be permitted as a method of tumor measurement. The same method used at baseline must be used consistently for tumor assessment and will be repeated every 6-8 weeks until disease progression. Confirmation of response will occur no less than 4 weeks from the first evidence of response. A bone and/or PET scan can be repeated per the investigator's discretion but must be repeated to confirm a complete response (CR) if bone lesions were present at baseline.

Patients may continue to receive study therapy unless unacceptable toxicity, disease progression, intercurrent illness or one of the criteria listed in 5.3 require discontinuation

For reasonable cause, either the Investigator or the Sponsor may terminate this study permanently. Written notification of the termination is required.

Conditions that may warrant termination include, but are not limited to:
The discovery of an unexpected significant or unacceptable risk to the patients enrolled in the study.
Failure of the Investigator to enter patients at an acceptable rate.
Insufficient adherence to protocol requirements (non-compliance).
Lack of evaluable and/or complete data.
Decision to modify the developmental plan of the drug.
A decision on the part of the Sponsor to suspend or discontinue development of the drug.

In the case that the trial is discontinued due to reasons other than unforeseen risk, patients who are currently receiving drug and are deriving benefit from the treatment may be allowed to continue receiving treatment.

Post discontinuation Period: Each enrolled patient will have a 30-day safety follow-up period which will occur 30 days after the last dose of study drug. The investigative sites will continue to monitor patients per routine clinical practice. Patients who complete treatment or discontinue without disease progression will continue to be evaluated for tumor response using the RECIST v1.1 guidelines (Eisenhauer et al. 2009, Appendix B) every 6-8 weeks until disease progression, death, or until study closure, whichever occurs first. The date of first documented disease progression must be recorded on the CRF even if progression occurs after the patient has started a new therapy. Monitoring for survival may also continue following progression on a monthly basis. Information will be collected regarding dates of disease progression, death and any post discontinuation systemic therapy, radiotherapy, or surgical intervention until the date of study closure.

Criteria for Removal from Treatment: The criteria for enrollment must be followed explicitly. If a patient who does not meet enrollment criteria is inadvertently enrolled, Lixte Biotechnology Holdings, Inc must be contacted. In addition, patients will be discontinued from the study drug and from the study in the following circumstances:
Enrollment in any other clinical trial involving an investigational product or enrollment in any other type of medical research judged not to be scientifically or medically compatible with this study.
Investigator/Physician Decision
   The investigator/physician decides that the patient should be withdraw from the study or study drug.
   If the patient, for any reason, requires treatment with another therapeutic agent that has been demonstrated to be effective for treatment of the study indication, discontinuation from the study drug occurs prior to introduction of the new agent.
Patient Decision
   The patient [or patient's designee (for example, parents or legal guardian)] requests to be withdrawn from the study or study drug.
Sponsor Decision
   The investigator or DSMB or Sponsor stops the study or stops the patient's participation in the study for medical, safety, regulatory, or other reasons consistent with applicable laws, regulations, and good clinical practice.
The patient is significantly noncompliant with study procedures and/or treatment
The patient has evidence of disease progression
Unacceptable toxicity
The patient becomes pregnant or fails to use adequate birth control (for those patients who are of childbearing potential).

Subject Follow-Up: The short-term safety follow-up period begins one day after the last dose of study drug and lasts 30 days. All AEs should be reported for a minimum of 30 days from the last dose of study drug. The long-term follow-up period begins after patients have either completed cycle 4 or have been discontinued from study drug and continues until disease progression or death. Patients may continue to be followed for survival following progression. The study will be considered complete following the data cutoff date and data lock for the final analysis. The statistical analysis will be performed after study completion.

Clinical Observations and Tests to be Performed
Efficacy: CT/PET/MRI scans
Safety: Adverse events (AEs) by CTCAE 5.0/serious adverse events (SAEs), clinical chemistry, hematology
Bioanalytical: Blood samples to measure plasma LB-100, endothall, and etoposide concentrations
Pharmacokinetic: LB-100 and etoposide exposure

Abbreviated Statistical Considerations

Safety: All patients who receive at least one dose of study drug will be evaluated for safety and toxicity. Safety analyses will include the following: summaries of the adverse event rates (including all events and study drug-related events), all serious adverse events (SAEs), deaths on-study, deaths within 30 days of the last dose of study drug, and discontinuations from study drug due to adverse events; listings and frequency tables categorizing laboratory and nonlaboratory adverse events by maximum CTCAE 5.0 grade and relationship to study drug.

Expanded Cohort: 12 patients at the RP2D will help confirm the choice of RP2D. If during the expansion cohort, more than 30% of the patients at initial RP2D experience a DLT, the study will hold accrual (accrual can also be held at the discretion of the PI for non-DLT or other safety considerations). With 12 patients, any serious treatment-related adverse event that occurs with a true frequency of 10%, will be observed at least once with a probability of 72%, and any such AE with a true frequency of 20% would be observed at least once with a probability of 93%. The DLT rate can be estimated with a standard error of at most 14%.

Prohibited: Any concomitant therapy intended for the treatment of cancer, whether health authority-approved or experimental, is prohibited for various time periods prior to starting study treatment, and during study treatment until disease progression is documented and patient has discontinued study treatment. This includes, but is not limited to, chemotherapy, hormonal therapy, immunotherapy, radiotherapy, investigational agents, or herbal therapy (unless otherwise noted).

The following medications are prohibited while on study, unless otherwise noted:
Traditional herbal medicines, because their use may result in unanticipated drug-drug interactions that may cause or confound assessment of toxicity
Denosumab; patients who are receiving denosumab prior to enrollment must be willing and eligible to receive a bisphosphonate instead while in the study
Any live, attenuated vaccine (e.g., FluMist®) within 28 days prior to first study drug, during treatment, or within 90 days following the last dose of atezolizumab
Use of steroids to premedicate patients for whom CT scans with contrast are contraindicated (i.e., patients with contrast allergy or impaired renal clearance); in such patients, non-contrast CT scans of the chest and non-contrast CT scans or MRIs of the abdomen and pelvis should be performed
Medications that are known to prolong the QT interval and/or are associated with a risk of Torsades de Pointes.
CYP450 substrates (see Appendix F).
Nephrotoxic compounds.
Warfarin.
Antiepileptic medications that may increase etoposide clearance (including but not limited to phenytoin, phenobarbital, carbamazepine, and valproic acid).
Strong P-glycoprotein inhibitors

Definition of Dose-Limiting Toxicity (DLT): The NCI Common Terminology Criteria for Adverse Events (CTCAE) Version 5.0 will be used to grade toxicity. Per section 5.5 GCSF is not allowed in Cycle 1, as it may suppress a toxicity that might otherwise occur. If a protocol deviation occurs and a patient does receive GCSF in Cycle 1, they will be considered inevaluable for DLT and replaced, unless they experience a DLT in Cycle 1. DLT is defined as any of the following adverse events occurring in the first cycle of treatment and considered to be possibly, probably, or definitely related to study treatment:
Nausea/vomiting of Grade 3 or greater despite maximal antiemetic therapy.
Any Grade 4 (immune-related adverse events (irAE)
Diarrhea of Grade 3 or greater despite maximal antidiarrheal therapy.
Any ≥Grade 3 colitis (infectious etiologies should have been ruled out and endoscopic verification is strongly encouraged)
Any Grade 3 or 4 noninfectious pneumonitis irrespective of duration
Any Grade 2 pneumonitis that does not resolve to ≤Grade 1 within 3 days of the initiation of maximal supportive care
Any Grade 3 irAE, excluding colitis or pneumonitis, that does not downgrade to Grade 2 within 3 days after onset of the event despite optimal medical management including systemic corticosteroids or does not downgrade to ≤Grade 1 or baseline within 14 days
Concurrent elevation of AST or ALT ≥3×ULN AND total bilirubin >2×ULN
AST or ALT >8×ULN or total bilirubin ≥3×ULN, even if asymptomatic, unless it is related to a definite progression of liver metastases or another clearly identifiable etiology.
Grade 4 neutropenia observed for greater than 5 days duration or Grade 3 neutropenia associated with fever of any duration or where sepsis results or Grade 3 neutropenia lasting >7 days.
Grade 4 thrombocytopenia or Grade 3 thrombocytopenia with clinically significant bleeding or Grade 3 thrombocytopenia lasting >7 days.
Grade 4 anemia.
Any ≥Grade 3 AE, except for the exclusions listed below:
   Grade 3 fatigue lasting ≤7 days
   Grade 3 laboratory abnormalities, other than ALT or AST, that are not considered clinically significant and that return to grade 2 or less within 72 hours
   Grade 3 endocrine disorder (thyroid, pituitary, and/or adrenal insufficiency) that is managed with or without systemic corticosteroid therapy and/or hormone replacement therapy and the subject is asymptomatic
   Grade 3 inflammatory reaction attributed to a local antitumor response (eg, inflammatory reaction at sites of metastatic disease, lymph nodes, etc.)
   Concurrent vitiligo or alopecia of any AE grade
   Grade 3 infusion-related reaction (first occurrence and in the absence of steroid prophylaxis) that resolves within 6 hours with appropriate clinical management
   Grade 3 or 4 lymphopenia

Dose Delays/Modifications for Adverse Events

Dose Modifications: It is anticipated that most of the treatment related toxicity on this trial will be caused by carboplatin/etoposide/atezolizumab. Myelosuppression, predominantly neutropenia, will occur frequently; common non-hematologic toxicities include fatigue, nausea, vomiting, and mucositis. In contrast, LB-100 is anticipated to be well tolerated; few toxicities observed in phase I overlapped the known toxicity profile of carboplatin, etoposide and atezolizumab. The following general dose modification rules will, therefore, be used for patients on the LB-100 treatment arm:

If the initiation of a cycle is delayed due to carboplatin/etoposide/atezolizumab toxicity, the LB-100 will also be delayed to begin concurrently with the carboplatin/etoposide/atezolizumab.

If atezolizumab is held then LB-100 should be held as well, as it is a potential immunomodulatory

If toxicity is typical of carboplatin/etoposide/atezolizumab and requires dose reductions, the dose of LB-100 should not be reduced.

If the toxicity is attributed specifically to one or two agents (carboplatin, etoposide, atezolizumab), the attributed agents will be dose reduced; otherwise, the doses of all 3 drugs should be reduced.

Patients who require a treatment delay of more than 28 days due to toxicity will be discontinued from the study. An exception is given for tapering of steroids. If a patient must be tapered off steroids used to treat adverse events, atezolizumab may be withheld until steroids are discontinued or reduced to prednisone dose (or dose equivalent)≤10 mg/day.

Carboplatin Etoposide Dose Modifications: Two dose reductions of carboplatin and etoposide are allowed. Patients who require dose reductions will not have re-escalation. If grade 3/4 toxicity reoccurs after 2 dose reductions have occurred, the offending agent or agents will be discontinued. If carboplatin, etoposide and atezolizumab must be discontinued due to toxicity, LB-100 will also be discontinued. Patients who require a treatment delay of more than 28 days due to toxicity will be discontinued from the study. Dose reductions for carboplatin and etoposide are shown in Table 4.

TABLE 4
Dose Reductions for Carboplatin & Etoposide

| Dose Level | Carboplatin (AUC) | Etoposide (mg/m²) |
|---|---|---|
| Starting Dose | 5.0 | 100 × 3 days |
| −1 | 4.5 | 75 × 3 days |
| −2 | 4.0 | 50 × 3 days |

Hematologic Toxicity: Dose adjustment will be based on the blood count measured on Day 1 (+/−2 days) of each cycle. No dose modifications will be based on nadir counts. See Table 5 below.

TABLE 5
Dose adjustments for carboplatin and for hematologic toxicity

| Blood Counts | Carboplatin (AUC) | Etoposide (mg m²) |
|---|---|---|
| ANC >1500/μL and | No dose modification | No dose modification |
| Platelets >100,000/μL | | |
| ANC <1500/μL or | Delay dose^{a} | Delay dose^{a} |
| Platelets <100,000/μL | Resume with one level dose | Resume with one level dose |
| | reduction. Consider the addition | reduction. Consider the addition |
| | of prophylactic G-CSF | of prophylactic G-CSF |
| Febrile neutropenia | Delay dose^{b} | Delay dose^{b} |
| (ANC ≤1000/μL | Resume with one level dose | Resume with one level dose |
| and Temp ≥101° F. | reduction. Consider the addition | reduction. Consider the addition |
| (38.5° C.)] | of prophylactic G-CSF | of prophylactic G-CSF |

^{a}Check counts at least weekly until ANC ≥1500/μL and platelets ≥100,000/, μL then proceed with Day 1 dose
^{b}Delay dose until the infection is adequately treated and blood counts are ANC ≥1500/μL and platelets ≥100,000/μL

Non-Hematologic Toxicity: If grade 3 or 4 non-hematologic toxicity occurs:
Delay treatment with all drugs
Make an assessment regarding which drug or drugs produced the toxicity
Reevaluate the patient at least once weekly until the toxicity resolves to ≤grade 1
Reduce the dose of the offending agent or agents by one dose level
If toxicity is irreversible or has not resolved to ≤grade 1 after a 3-week treatment delay, the patient should be removed from the study
Creatinine clearance (Cockcroft and Gault formula) should be ≥45 mL/min prior to the start of any cycle.

Atezolizumab Dose Holding: There will be no dose reduction for atezolizumab, but patients may temporarily suspend treatment with atezolizumab for up to 4 weeks beyond the last dose if they experience an adverse event that requires a dose to be held. An exception is given for tapering of steroids. If a patient must be tapered off steroids used to treat adverse events, atezolizumab may be withheld until steroids are discontinued or reduced to prednisone dose (or dose equivalent)≤10 mg/day.

Management of Atezolizumab-Specific Adverse Events: Additional tests, such as autoimmune serology or biopsies, should be used to determine a possible immunogenic etiology. Although most immune-mediated adverse events observed with immunomodulatory agents have been mild and self-limiting, such events should be recognized early and treated promptly to avoid potential major complications. Discontinuation of atezolizumab may not have an immediate therapeutic effect and, in severe cases, immune-mediated toxicities may require acute management with topical corticosteroids, systemic corticosteroids or other immunosuppressive agents.

TABLE 6
Adverse Events
Atezolizumab AE Management and Dose Interruption Guidelines for Specific Toxicities

| Toxicity | Severity/Duration | Management |
|---|---|---|
| Abdominal pain | Acute Abdominal pain | Symptoms of abdominal pain associated with elevations of |
| | | amylase and lipase, suggestive of pancreatitis, have been |
| | | associated with administration of other immunomodulatory |
| | | agents. The differential diagnosis of acute abdominal pain |
| | | should include pancreatitis. |
| | | Appropriate workup should include an evaluation for |
| | | obstruction, as well as serum amylase and lipase tests. See the |
| | | guidelines for “Amylase and/or lipase increase” and |
| | | “Immune-related pancreatitis” elsewhere in this table, as |
| | | needed. |
| | | Right upper-quadrant abdominal pain and/or unexplained |
| | | nausea or vomiting should be evaluated for potential |
| | | hepatotoxicity (see the “Hepatotoxicity” guideline elsewhere |
| | | in this table). |
| Adrenal insufficiency | Grade 2+ | Hold atezolizumab. (symptomatic) |
| | | Consider referral of patient to endocrinologist. |
| | | Perform appropriate imaging. |
| | | Initiate treatment with 1-2 mg/kg/day intravenous |
| | | methylprednisolone or equivalent and convert to 1-2 |
| | | mg/kg/day oral prednisone or equivalent upon improvement. |
| | | If event resolves to Grade 1 or better and patient is stable on |
| | | replacement therapy (if required) within 4 weeks, taper |
| | | corticosteroids over ≥1 month and resume atezolizumab. |
| | | Permanently discontinue atezolizumab if event does not |
| | | resolve to Grade 1 or better or patient is not stable on |
| | | replacement therapy within 4 weeks. |
| Amylase and/or | Grade 1 | Continue atezolizumab. |
| lipase increased | | Monitor amylase and lipase prior to dosing. |
| | Grade 2 | Continue atezolizumab. |
| | | Monitor amylase and lipase weekly. |
| | | For prolonged elevation (e.g., >3 weeks), consider treatment |
| | | with 10 mg/day oral prednisone or equivalent |
| | Grade 3 or 4 | Hold atezolizumab. |
| | | Consider referral of patient to gastrointestinal (GI) specialist. |
| | | Monitor amylase and lipase every other day. |
| | | If no improvement, consider treatment with 1-2 mg/kg/day |
| | | oral prednisone or equivalent. |
| | | If event resolves to Grade 1 or better within 4 weeks, taper |
| | | corticosteroids over ≥1 month and resume atezolizumab. |
| | | Permanently discontinue atezolizumab if event does not |
| | | resolve to Grade 1 or better within 4 weeks. |
| | | For recurrent events, permanently discontinue atezolizumab. |
| Dermatologic | Grade 1 | Continue atezolizumab. |
| toxicity/rash (e.g., | | Consider topical steroids and/or other symptomatic therapy |
| maculopapular or | | (e.g., antihistamines). |
| purpura) | | |
| | Grade 2 | Continue atezolizumab. Consider dermatologist referral. |
| | | Administer topical corticosteroids. |
| | Grade 3 | Hold atezolizumab. |
| | | Refer patient to dermatologist. Administer oral prednisone 10 |
| | | mg or equivalent. If the event does not improve within 48-72 |
| | | hours, increase dose to 1-2 mg/kg/day or equivalent. Restart |
| | | atezolizumab if event resolves to Grade 1 or better within 4 |
| | | weeks. |
| | | Permanently discontinue atezolizumab if event does not |
| | | resolve to Grade 1 or better within 4 weeks. |
| | Grade 4 | Permanently discontinue atezolizumab. |
| | | Patient may not resume treatment, regardless of benefit. |
| | | Otherwise, manage as above. |
| | Persistent and/or severe | A dermatologist should evaluate the event. A biopsy should |
| | rash or pruritus, any | be performed, unless contraindicated. |
| | grade | |
| Diarrhea or colitis | Any grade | Patients should be advised to inform the investigator if any |
| | | diarrhea occurs, even if it is mild. |
| | | All events of diarrhea or colitis should be thoroughly |
| | | evaluated for other more common etiologies. For events of |
| | | significant duration or magnitude or associated with signs of |
| | | systemic inflammation or acute-phase reactants (e.g., |
| | | increased CRP, platelet count, or bandemia): Perform |
| | | sigmoidoscopy (or colonoscopy, if appropriate) with colonic |
| | | biopsy, with three to five specimens for standard paraffin |
| | | block to check for inflammation and lymphocytic infiltrates to |
| | | confirm colitis diagnosis. |
| | Grade 1 | Continue atezolizumab. |
| | | Initiate symptomatic treatment. |
| | | Endoscopy is recommended if symptoms persist for >7 days. |
| | | Monitor closely |
| | Grade 2 | Hold atezolizumab. |
| | | Initiate symptomatic treatment. |
| | | Patient referral to GI specialist is recommended. |
| | | For recurrent events or events that persist >5 days, initiate |
| | | treatment with 1-2 mg/kg/day oral prednisone or equivalent. |
| | | If event resolves to Grade 1 or better within 4 weeks, taper |
| | | corticosteroids over ≥1 month and resume atezolizumab. |
| | | Permanently discontinue atezolizumab if event does not |
| | | resolve to Grade 1 or better within 4 weeks. Resumption of |
| | | atezolizumab may be considered, after consultation with the |
| | | trial PI, in patients who are deriving benefit and have fully |
| | | recovered from the immune-related event. |
| | Grade 3 | Hold atezolizumab. |
| | | Refer patient to GI specialist for evaluation and confirmatory |
| | | biopsy. |
| | | Initiate treatment with 1-2 mg/kg/day intravenous |
| | | methylprednisolone or equivalent and convert to 1-2 |
| | | mg/kg/day oral prednisone or equivalent upon improvement. |
| | | If event resolves to Grade 1 or better within 4 weeks, taper |
| | | corticosteroids over ≥1 month and resume atezolizumab. |
| | | Permanently discontinue atezolizumab if event does not |
| | | resolve to Grade 1 or better within 4 weeks. Resumption of |
| | | atezolizumab may be considered, after consultation with the |
| | | Principal Investigator, in patients who are deriving benefit |
| | | and have fully recovered from the immune-related event. |
| | Grade 4 | Permanently discontinue atezolizumab. Patient may not |
| | | resume treatment, resardless of benefit. |
| | | Refer patient to GI specialist for evaluation and confirmation |
| | | biopsy. |
| | | Initiate treatment with 1-2 mg/kg/day intravenous |
| | | methylprednisolone or equivalent and convert to 1-2 |
| | | mg/kg/day oral prednisone or equivalent upon improvement. |
| | | If event does not improve within 48 hours after initiating |
| | | corticosteroids, consider adding an immunosuppressive agent. |
| | | If event resolves to Grade 1 or better, taper corticosteroids |
| | | over ≥1 month. |
| Hepatotoxicity | Right upper-abdominal | Risk of immune-mediated hepatitis. LFTs should be |
| | pain &/or nausea or | performed immediately, and LFTs should be reviewed before |
| | vomiting | administration of the next dose of study drug. For patients |
| | | with unexplained elevated LFTs, concurrent medication, viral |
| | | hepatitis, and toxic or neoplastic etiologies should be |
| | | considered and addressed, as appropriate. |
| | | Symptoms of abdominal pain associated with elevations of |
| | | amylase and lipase, suggestive of pancreatitis, have been |
| | | associated with the administration of atezolizumab. The |
| | | differential diagnosis of acute abdominal pain should also |
| | | include pancreatitis, as described below. |
| | Grade 1 hepatic event | Continue atezolizumab. |
| | | Monitor LFTs until values resolve to within normal limits. |
| | Grade 2 hepatic | Continue atezolizumab. |
| | event, ≤5 days | Monitor LFTs more frequently until values resolve to baseline |
| | | values. |
| | Grade 2 hepatic | Hold atezolizumab. |
| | event, >5 days | Initiate treatment with 1-2 mg/kg/day oral prednisone or |
| | | equivalent. |
| | | If event resolves to Grade 1 or better within 4 weeks, taper |
| | | corticosteroids over ≥1 month and resume atezolizumab. |
| | | Permanently discontinue atezolizumab if event does not |
| | | resolve to Grade 1 or better within 4 weeks. |
| | Grade 3 or 4 | Permanently discontinue atezolizumab. |
| | hepatic event | Consider patient referral to GI specialist for evaluation and |
| | | liver biopsy to establish etiology of hepatic injury. |
| | | Initiate treatment with 1-2 mg/kg/day oral prednisone or |
| | | equivalent. |
| | | If event does not improve within 48 hours after initiating |
| | | corticosteroids, consider adding an immunosuppressive agent. |
| | | If event resolves to Grade 1 or better, taper corticosteroids |
| | | over ≥1 month. Continue atezolizumab. |
| Hyperglycemia | Grade 1 or 2 | Initiate treatment with insulin if needed. |
| | | Monitor for glucose control. |
| | Grade 3 or 4 | Hold atezolizumab. |
| | | Initiate treatment with insulin. |
| | | Monitor for glucose control. |
| | | Resume atezolizumab when symptoms resolve and glucose |
| | | levels are stable. |
| Hyperthyroidism | Grade 1 | TSH ≥0.1 mU/L and <0.5 mU/L: Continue atezolizumab. |
| | (asymptomatic) | Monitor TSH every 4 weeks. |
| | | TSH <0.1 mU/L: Follow guidelines for symptomatic |
| | | hyperthyroidism. |
| | Grade 2+ | Hold atezolizumab. |
| | (symptomatic) | Initiate treatment with anti-thyroid drug such as methimazole |
| | | or carbimazole as needed. |
| | | Consider patient referral to endocrinologist. |
| | | Resume atezolizumab when symptoms are controlled and |
| | | thyroid function is improving. |
| | | Permanently discontinue atezolizumab for life-threatening |
| | | immune-related hyperthyroidism. |
| Hypothyroidism | Grade 1 | Continue atezolizumab. |
| | (asymptomatic) | Start thyroid-replacement hormone. |
| | | Monitor TSH weekly. |
| | Grade 2+ | Hold atezolizumab. |
| | (symptomatic) | Start thyroid-replacement hormone. Consider referral to an |
| | | endocrinologist. |
| | | Monitor TSH weekly. |
| | | Restart atezolizumab when symptoms are controlled and |
| | | thyroid function is improving |
| Meningo- | All grades | Permanently discontinue atezolizumab. Patient may not |
| encepahlitis, | | resume treatment, resardless of benefit. |
| immune-related/ | | Refer patient to neurologist. |
| (signs and symptoms | | Initiate treatment with 1-2 mg/kg/day IV methylprednisolone |
| in absence of an | | or equivalent and convert to 1-2 mg/kg/day oral prednisone or |
| identified alternate | | equivalent upon improvement. |
| etiology) | | If event resolves to Grade 1 or better, taper corticosteroids |
| | | over ≥1 month. |
| | | If event does not improve within 48 hours after initiating |
| | | corticosteroids, consider adding an immunosuppressive agent. |
| Myasthenia gravis | All grades | Permanently discontinue atezolizumab. Patient may not |
| and Guillain-Barré | | resume treatment, regardless of benefit. |
| syndrome | | Refer patient to neurologist. |
| | | Initiate treatment as per institutional guidelines. |
| | | Consider initiation of 1-2 mg/kg/day oral or IV prednisone or |
| | | equivalent. |
| Myocarditis | All grades | Permanently discontinue atezolizumab. Patient may not |
| | | resume treatment, regardless of benefit. |
| Nephritis | Grade 2 | Withhold atezolizumab. |
| | | Refer patient to renal specialist and consider renal biopsy and |
| | | supportive measures as indicated. Corticosteroids and/or |
| | | additional immunosuppressive agents should be administered |
| | | as clinically indicated. |
| | | If event resolves to Grade 1 or better within 4 weeks, taper |
| | | corticosteroids over ≥1 month and resume atezolizumab. |
| | Grade 3-4 | Permanently discontinue atezolizumab. |
| | | Refer patient to renal specialist and consider renal biopsy and |
| | | supportive measures as indicated. Corticosteroids and/or |
| | | additional immunosuppressive agents should be administered |
| | | as clinically indicated. |
| Neuropathy, | Grade 1 | Continue atezolizumab. |
| immune-related | | Evaluate for alternative etiologies. |
| (sensory and/or | | |
| motor) | | |
| | Grade 2 | Hold atezolizumab. |
| | | Evaluate for alternative etiologies. |
| | | Initiate treatment as per institutional guidelines. |
| | | Resume atezolizumab if event resolves to Grade 1 or better |
| | | within 4 weeks. |
| | Grade 3 or 4 | Permanently discontinue atezolizumab. |
| | | Initiate treatment as per institutional guidelines. |
| Ocular event (e.g., | Grade 1 | Continue atezolizumab. |
| uveitis, retinal events | | Patient referral to ophthalmologist is strongly recommended. |
| | | Initiate treatment with topical corticosteroid eye drops and |
| | | topical immunosuppressive therapy. |
| | | If symptoms persist, treat as a Grade 2 event. |
| | Grade 2 | Withhold atezolizumab. |
| | | Patient referral to ophthalmologist is strongly recommended. |
| | | Initiate treatment with topical corticosteroid eye drops and |
| | | topical immunosuppressive therapy. |
| | | If event resolves to Grade 1 or better within 4 weeks, taper |
| | | corticosteroids over ≥1 month and resume atezolizumab. |
| | | Permanently discontinue atezolizumab if event does not |
| | | resolve to Grade 1 or better within 4 weeks. |
| | Grade 3 or 4 | Permanetly discontinue atezolizumab. |
| | | Refer patient to ophthalmologist. |
| | | Initiate treatment with 1-2 mg/kg/day oral prednisone or |
| | | equivalent. |
| | | If event resolves to Grade 1 or better, taper corticosteroids |
| | | over ≥1 month. For Grade 3 AEs, patient may only resume |
| | | treatment after consultation with the trial PI; for Grade 4, |
| | | patient cannot resume treatment, regardless of benefit. |
| Pancreatitis, immune | Grade 2 or 3 | Hold atezolizumab. |
| related | | Refer patient to GI specialist. |
| | | Initiate treatment with 1-2 mg/kg/day intravenous |
| | | methylprednisolone or equivalent and convert to 1-2 |
| | | mg/kg/day oral prednisone or equivalent upon improvement. |
| | | If event resolves to Grade 1 or better within 4 weeks, taper |
| | | corticosteroids over ≥1 month and resume atezolizumab. |
| | | Permanently discontinue atezolizumab if event does not |
| | | resolve to Grade 1 or better within 4 weeks. Patient may only |
| | | resume treatment after consultation with the trial PI. |
| | | For recurrent events, permanently discontinue atezolizumab. |
| | | Patient may not resume treatment, regardless of benefit. |
| | Grade 4 | Permanently discontinue atezolizumab. Patient may not |
| | | resume treatment, regardless of benefit. |
| | | Refer patient to GI specialist. |
| | | Initiate treatment with 1-2 mg/kg/day intravenous |
| | | methylprednisolone or equivalent and convert to 1-2 |
| | | mg/kg/day oral prednisone or equivalent upon improvement. |
| | | If event does not improve within 48 hours after initiating |
| | | corticosteroids, consider adding an immunosuppressive agent. |
| | | If event resolves to Grade 1 or better, taper corticosteroids |
| | | over ≥1 month. |
| Pulmonary toxicity | All events | Evaluate thoroughly for other commonly reported etiologies |
| | | such as pneumonia/infection, lymphangitic carcinomatosis, |
| | | pulmonary embolism, heart failure, chronic obstructive |
| | | pulmonary disease (COPD), or pulmonary hypertension. |
| | Grade 1 | Continue atezolizumab and monitor closely. |
| | | Re-evaluate on serial imaging. |
| | | Consider patient referral to a pulmonary specialist. |
| | | For recurrent pneumonitis, treat as a Grade 3 or 4 event. |
| | Grade 2 | Hold atezolizumab. |
| | | Refer patient to pulmonary and infectious disease specialists |
| | | and consider bronchoscopy or bronchoscopic alveolar lavage |
| | | (BAL). |
| | | Initiate treatment with 1-2 mg/kg/day oral prednisone or |
| | | equivalent. |
| | | If event resolves to Grade 1 or better within 4 weeks, taper |
| | | corticosteroids over ≥1 month and resume atezolizumab. |
| | Grade 3 or 4 | Hold atezolizumab. |
| | | Bronchoscopy or BAL is recommended. |
| | | Initiate treatment with 1-2 mg/kg/day oral prednisone or |
| | | equivalent. |
| | | If event does not improve within 48 hours after initiating |
| | | corticosteroids, consider adding an immunosuppressive agent. |
| | | If event resolves to Grade 1 or better, taper corticosteroids |
| | | over ≥1 month. For Grade 3 AEs, patient may only resume |
| | | treatment after consultation with the Principal Investigator; |
| | | for Grade 4, patient cannot resume treatment, regardless of |
| | | benefit. |

Systemic Immune Activation: Systemic immune activation is a rare condition characterized by an excessive immune response. Given the mechanism of action of atezolizumab, systemic immune activation is considered a potential risk. Systemic immune activation should be included in the differential diagnosis for patients who, in the absence of an alternative etiology, develop a sepsis-like syndrome after administration of atezolizumab, and the initial evaluation should include the following:
CBC with peripheral smear
PT, PTT, fibrinogen, and D-dimer
Ferritin
Triglycerides
AST, ALT, and total bilirubin
LDH
Complete neurologic and abdominal examination (assess for hepatosplenomegaly)

LB-100 Dose Modifications: Two dose reductions of LB-100 are allowed. Re-escalation is allowed once at the discretion of the investigator. Patients with a delay of more than 21 days of LB-100 must be discontinued from study therapy. If grade 3/4 toxicity attributed to LB-100 occurs after 2 previous dose reductions, LB-100 will be discontinued. Patients who are benefiting from treatment may continue carboplatin/etoposide/atezolizumab. Dose reductions of LB-100 are outlined in Table 7.

TABLE 7
LB-100 Dose Levels

| Dose Level | LB-100 Dose |
|---|---|
| −2 | 0.50 mg/m² |
| −1 | 0.83 mg/m² |
| Starting | 1.25 mg/m² |
| +1 | 1.75 |
| +2 | 2.13 |
| +3 | 3.10 |

Hematologic Toxicity: Myelosuppression may infrequently occur with LB-100. Therefore, if grade 3/4 myelosuppression occurs, for the first occurrence the doses of carboplatin and etoposide will be reduced, but LB-100 will stay the same. For the second occurrence of Grade 3/4 myelosuppression LB-100 will be reduced. Atezolizumab will be delayed or discontinued if autoimmune cyctopenias occur. There were no notable adverse events reported in the Phase I trial and we do not expect dose reductions or interruptions.

Non-hematologic Toxicity: The non-hematologic toxicity attributed to LB-100 should be managed as outlined in Table 8.

TABLE 8
Dose adjustments of LB-100

| Toxicity | Management | | Dose Reduction |
|---|---|---|---|
| Injection Site Reaction, grade 3 | 1.) | Interrupt LB-100 | Reduce 1 dose level |
| | 2.) | Administer topical treatment as necessary | |
| Grade 2 Nephrotoxicity | 1.) | Interrupt LB-100 | Prolong infusion time to 2 |
| | 2.) | Reexamine patient at least weekly until | hours. |
| | | toxicity improved to ≤ grade 1 | |
| Grade 3 or 4 Nephrotoxicity | 1.) | Interrupt LB-100 | Reduce 1 dose level and prolong |
| | 2.) | Reexamine patient at least weekly until | infusion time to 2 hours. |
| | | toxicity improved to ≤ grade 1 | |
| Other Grade 2 clinically | 1.) | Interrupt LB-100 | First occurrence: Maintain Dose |
| significant non-hematologic | 2.) | Reexamine patient at least weekly until | Second occurrence: Reduce 1 |
| toxicity* | | toxicity improved to ≤ grade 1 | dose level |
| Other Grade 3-4 clinically | 1.) | Interrupt LB-100 | Reduce 1 dose level |
| significant non-hematologic | 2.) | Reexamine patient at least weekly until | |
| toxicity* | | toxicity improved to ≤ grade 1 | |
| Any toxicity requiring a hold of | 1.) | Interrupt LB-100) | Maintain dose level. |
| atezolizumab | 2.) | Reexamine patient at least weekly until | |
| | | atezolizumab can be restarted | |

*Alopecia, and clinically insignificant lab abnormalities are examples of things that would not be considered clinically significant

Pharmacokinetic Studies: Plasma for pharmacokinetic (PK) measurements of LB-100, its major metabolite endothall will be collected in all patients according the sample schedule shown in Table 9. The sampling schedule allows for determination of LB-100 and endothall PK when LB-100 is given prior to etoposide (Day 1) and when it is given together with etoposide (Day 3). Etoposide PK will also be assessed in patients in the expanded MTD cohort both alone (Day 2) and in combination with LB-100 (Day 3). For measurement of LB-100 and endothall, 5 mL of venous blood will be drawn into a chilled heparin collection tube (sodium or lithium) and kept on ice until the plasma is separated. Plasma will be aliquoted (two aliquots) into appropriately labeled polypropylene tubes (1.8-2 mL cryovials) containing 0.5N NaOH. For every 1.0 mL of plasma aliquoted 0.1 mL of 0.5N NaOH is to be added. Samples will be stored at −70° C. until the time of shipment. For measurement of etoposide, an additional 4 mL of venous blood will be drawn into EDTA-containing collection tubes at the times indicated in Table 9. Tubes will be kept on ice until plasma is separated and aliquoted into appropriately labeled cryovials and stored at <−70° C. for subsequent batch analysis.

TABLE 9
Pharmacokinetic Sample Schedule

| | | One (1) 5 mL heparin | |
|---|---|---|---|
| | | tube for LB-100 and | One (1) 4 mL EDTA |
| Study Day | Draw Time | endothall | tube for etoposide* |
| Day 1 | pre-dose | X | |
| | immediately at end of LB-100 infusion | X | |
| | 15 minutes (±5 minutes) post LB-100 | X | |
| | infusion | | |
| | 30 minutes (±5 minutes) post LB-100 | X | |
| | infusion | | |
| | 1 hour (±15 minutes) post LB-100 | X | |
| | infusion | | |
| | 2 hours (±15 minutes) post LB-100 | X | |
| | infusion | | |
| | 4 hours (±30 minutes) post LB-100 | X | |
| | infusion and prior to etoposide. | | |
| Day 2 | Pre-treatment (24 hours (±60 minutes) | X | X* |
| | post LB-100 infusion on day 1) | | |
| | immediately prior to the end of etoposide | | X* |
| | infusion | | |
| | 2 hours (±30 minutes) post etoposide | | X* |
| | infusion | | |
| | 6 hours (±30 minutes) post etoposide | | X* |
| | infusion | | |
| Day 3 | Pre-treatment [48 hours (±60 minutes) | X | X* |
| | post LB-100 infusion on day 1 and 24 | | |
| | hours (±60 minutes) etoposide infusion | | |
| | on day 2] | | |
| | immediately at end of LB-100 infusion | X | |
| | 15 minutes (±5 minutes) post LB-100 | X | |
| | infusion | | |
| | 30 minutes (±5 minutes) post LB-100 | X | |
| | infusion | | |
| | 1 hour (±15 minutes) post LB-100 and | X | X* |
| | pre etoposide | | |
| | 2 hours (±15 minutes) post LB-100 and | X | X* |
| | immediately prior to end of etoposide | | |
| | infusion | | |
| | 3 hours (±30 minutes) post LB-100 and 1 | X | |
| | hours (±30 minutes) post etoposide | | |
| | 4 hours (±30 minutes) post LB-100 and 2 | X | X* |
| | hours (±30 minutes) post etoposide | | |
| | 8 hours (±30 minutes) post LB-100 and 6 | X | X* |
| | hours (±30 minutes) post etoposide | | |
| Day 4 | Post-treatment [24 hours (±60 minutes) | X | X* |
| | post LB-100 and 22 hours (±60 minutes) | | |
| | post etoposide on day 3] | | |

*Samples for etoposide PK will be collected only in patients enrolled in the expanded MTD cohort.

Pharmacokinetic Data Analysis: Plasma PK data will be analyzed using both non-compartmental and compartmental methods to derive the relevant secondary PK parameters. Non-compartmental PK methods will be used to determine the parameters (e.g. Cₘₐₓ, Tₘₐₓ t1/2, AUC₀₋ₜ, and CL) for LB-100 and its major metabolite endothall. Compartmental PK analyses of the etoposide data will be performed using ADAPT 5 software (USC Biomedical Simulations Resource, Los Angeles Calif.), and secondary PK parameters (e.g. CL_{sys}, V_{d}, t_{1/2}, AUC₀₋ₒₒ) will be determined for each individual. Individual non-compartmental and compartmental PK parameters for each drug and metabolite will be summarized, and potential exposure-response relationships for both safety and efficacy will be assessed.

Results: Results for a first study subject are as follows. A partial objective response (47%) was noted after the 2nd cycle at dose level 1 of LB-100 (0.83 mg/m2 day dl & 3) and this response improved to a 58% decrease in measurable tumor following the 4th and last cycle of induction therapy. Toxicity was not dose limiting and not greater than would be expected for the standard three drug combination without LB-100. Maintenance therapy with Atezolizumab and LB-100 is anticipated.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

### References (Example 2)

1. Quoix E, Breton J L, Daniel C, Jacoulet P, Debieuvre D, Paillot N, Kessler R, Moreau L, Coetmeur D, Lemarie E, Milleron B. Etoposide phosphate with carboplatin in the treatment of elderly patients with small-cell lung cancer: a phase II study. Ann Oncol. 2001; 12(7):957-62. Epub 2001/08/28. PubMed PMID: 11521802.
2. Maghfoor I, Perry M C. Lung cancer. Ann Saudi Med. 2005; 25(1):1-12. Epub 2005/04/13. PubMed PMID: 15822487; PMCID: PMC6150570.
3. Niell H B, Perry M C, Clamon G, Crawford J, Miller A A, Herndon J, 2nd, Green M R. Carboplatin/etoposide/paclitaxel in the treatment of patients with extensive small-cell lung cancer. Clin Lung Cancer. 2001; 2(3):204-9. Epub 2004/01/01. PubMed PMID: 14700479.
4. Sundstrom S, Bremnes R M, Kaasa S, Aasebo U, Hatlevoll R, Dahle R, Boye N, Wang M, Vigander T, Vilsvik J, Skovlund E, Hannisdal E, Aamdal S, Norwegian Lung Cancer Study G. Cisplatin and etoposide regimen is superior to cyclophosphamide, epirubicin, and vincristine regimen in small-cell lung cancer: results from a randomized phase III trial with 5 years' follow-up. J Clin Oncol. 2002; 20(24):4665-72. Epub 2002/12/19. doi: 10.1200/JCO.2002.12.111. PubMed PMID: 12488411.
5. Niell H B, Herndon J E, 2nd, Miller A A, Watson D M, Sandler A B, Kelly K, Marks R S, Perry M C, Ansari R H, Otterson G, Ellerton J, Vokes E E, Green M R, Cancer, Leukemia G. Randomized phase III intergroup trial of etoposide and cisplatin with or without paclitaxel and granulocyte colony-stimulating factor in patients with extensive-stage small-cell lung cancer: Cancer and Leukemia Group B Trial 9732. J Clin Oncol. 2005; 23(16):3752-9. doi: 10.1200/JCO.2005.09.071. PubMed PMID: 15923572.
6. Eckardt J R, von Pawel J, Papai Z, Tomova A, Tzekova V, Crofts T E, Brannon S, Wissel P, Ross G. Open-label, multicenter, randomized, phase III study comparing oral topotecan/cisplatin versus etoposide/cisplatin as treatment for chemotherapy-naïve patients with extensive-disease small-cell lung cancer. J Clin Oncol. 2006; 24(13):2044-51. Epub 2006/05/02. doi: 10.1200/JCO.2005.03.3332. PubMed PMID: 16648504.
7. Horn L, Mansfield A S, Szczesna A, Havel L, Krzakowski M, Hochmair M J, Huemer F, Losonczy G, Johnson M L, Nishio M, Reck M, Mok T, Lam S, Shames D S, Liu J, Ding B, Lopez-Chavez A,
Kabbinavar F, Lin W, Sandler A, Liu S V, Group I M S. First-Line Atezolizumab plus Chemotherapy in Extensive-Stage Small-Cell Lung Cancer. N Engl J Med. 2018; 379(23):2220-9. doi: 10.1056/NEJMoa1809064. PubMed PMID: 30280641.
8. Bunn P A, Jr. Review of therapeutic trials of carboplatin in lung cancer. Semin Oncol. 1989; 16(2 Suppl 5):27-33. Epub 1989/04/01. PubMed PMID: 2541506.
9. Bunn P A, Jr., Kelly K. A phase I study of carboplatin and paclitaxel in non-small cell lung cancer: a University of Colorado Cancer Center study. Semin Oncol. 1995; 22(4 Suppl 9):2-6. Epub 1995/08/01. PubMed PMID: 7644924.
10. Prendiville J, Lorigan P, Hicks F, Leahy B, Stout R, Burt P, Thatcher N. Therapy for small cell lung cancer using carboplatin, ifosfamide, etoposide (without dose reduction), mid-cycle vincristine with thoracic and cranial irradiation. Eur J Cancer. 1994; 30A(14):2085-90. Epub 1994/01/01. PubMed PMID: 7857708.
11. Gatzemeier U, Hossfeld D K, Neuhauss R, Reck M, Achterrath W, Lenaz L. Phase II and III studies with carboplatin in small cell lung cancer. Semin Oncol. 1992; 19(1 Suppl 2):28-36. Epub 1992/02/01. PubMed PMID: 1329220.
12. Larive S, Bombaron P, Riou R, Fournel P, Perol M, Lena H, Dussopt C, Philip-Joet F, Touraine F, Lecaer H, Souquet P J, Groupe Lyon-Saint Etienne d'Oncologie T. Carboplatin-etoposide combination in small cell lung cancer patients older than 70 years: a phase II trial. Lung Cancer. 2002; 35(1):1-7. Epub 2001/12/26. PubMed PMID: 11750705.
13. Perrotti D, Neviani P. Protein phosphatase 2A: a target for anticancer therapy. Lancet Oncol. 2013; 14(6):e229-38. Epub 2013/05/04. doi: 10.1016/S1470-2045(12)70558-2. PubMed PMID: 23639323; PMCID: PMC3913484.
14. Hong C S, Ho W, Zhang C, Yang C, Elder J B, Zhuang Z. LB100, a small molecule inhibitor of PP2A with potent chemo- and radio-sensitizing potential. Cancer Biol Ther. 2015; 16(6):821-33. Epub 2015/04/22. doi: 10.1080/15384047.2015.1040961. PubMed PMID: 25897893; PMCID: PMC4623051.
15. Chung V, Mansfield A S, Braiteh F, Richards D, Durivage H, Ungerleider R S, Johnson F, Kovach J S. Safety, Tolerability, and Preliminary Activity of LB-100, an Inhibitor of Protein Phosphatase 2A, in Patients with Relapsed Solid Tumors: An Open-Label, Dose Escalation, First-in-Human, Phase I Trial. Clin Cancer Res. 2017; 23(13):3277-84. Epub 2017/01/01. doi: 10.1158/1078-0432.CCR-16-2299. PubMed PMID: 28039265.
16. Lu J, Kovach J S, Johnson F, Chiang J, Hodes R, Lonser R, Zhuang Z. Inhibition of serine/threonine phosphatase PP2A enhances cancer chemotherapy by blocking DNA damage induced defense mechanisms. Proc Natl Acad Sci USA. 2009; 106(28):11697-702. Epub 2009/07/01. doi: 10.1073/pnas.0905930106. PubMed PMID: 19564615; PMCID: PMC2710674.
17. Wang Y, Yang R, Gu J, Yin X, Jin N, Xie S, Wang Y, Chang H, Qian W, Shi J, Iqbal K, Gong C X, Cheng C, Liu F. Cross talk between PI3K-AKT-GSK-3beta and PP2A pathways determines tau hyperphosphorylation. Neurobiol Aging. 2015; 36(1):188-200. Epub 2014/09/16. doi: 10.1016/j.neurobiolaging.2014.07.035. PubMed PMID: 25219467.
18. Liu G P, Wei W, Zhou X, Shi H R, Liu X H, Chai G S, Yao X Q, Zhang J Y, Peng C X, Hu J, Li X C, Wang Q, Wang J Z. Silencing PP2A inhibitor by lenti-shRNA interference ameliorates neuropathologies and memory deficits in tg2576 mice. Mol Ther. 2013; 21(12):2247-57. Epub 2013/08/08. doi: 10.1038/mt.2013.189. PubMed PMID: 23922015; PMCID: PMC3863796.
19. Gordon I K, Lu J, Graves C A, Huntoon K, Frerich J M, Hanson R H, Wang X, Hong C S, Ho W, Feldman M J, Ikejiri B, Bisht K, Chen X S, Tandle A, Yang C, Arscott W T, Ye D, Heiss J D, Lonser R R, Camphausen K, Zhuang Z. Protein Phosphatase 2A Inhibition with LB100 Enhances Radiation-Induced Mitotic Catastrophe and Tumor Growth Delay in Glioblastoma. Mol Cancer Ther. 2015; 14(7):1540-7. Epub 2015/05/06. doi: 10.1158/1535-7163.MCT-14-0614. PubMed PMID: 25939762; PMCID: PMC4497833.
20. Zhu X N, Chen L P, Bai Q, Ma L, Li D C, Zhang J M, Gao C, Lei Z N, Zhang Z B, Xing X M, Liu C X, He Z N, Li J, Xiao Y M, Zhang A H, Zeng X W, Chen W. PP2A-AMPKalpha-HSF1 axis regulates the metal-inducible expression of HSPs and ROS clearance. Cell Signal. 2014; 26(4):825-32. Epub 2014/01/15. doi: 10.1016/j.cellsig.2014.01.002. PubMed PMID: 24412756.
21. Xiao G, Chan L N, Klemm L, Braas D, Chen Z, Geng H, Zhang Q C, Aghajanirefah A, Cosgun K N, Sadras T, Lee J, Mirzapoiazova T, Salgia R, Ernst T, Hochhaus A, Jumaa H, Jiang X, Weinstock D M, Graeber T G, Muschen M. B-Cell-Specific Diversion of Glucose Carbon Utilization Reveals a Unique Vulnerability in B Cell Malignancies. Cell. 2018; 173(2):470-84 e18. Epub 2018/03/20. doi: 10.1016/j.cell.2018.02.048. PubMed PMID: 29551267; PMCID: PMC6284818.
22. Zhuang Z, Lu J, Lonser R, Kovach J S. Enhancement of cancer chemotherapy by simultaneously altering cell cycle progression and DNA-damage defenses through global modification of the serine/threonine phospho-proteome. Cell Cycle. 2009; 8(20):3303-6. Epub 2009/10/07. doi: 10.4161/cc.8.20.9689. PubMed PMID: 19806030.
23. Lu J, Zhuang Z, Song D K, Mehta G U, Ikejiri B, Mushlin H, Park D M, Lonser R R. The effect of a PP2A inhibitor on the nuclear receptor corepressor pathway in glioma. J Neurosurg. 2010; 113(2):225-33. Epub 2009/12/17. doi: 10.3171/2009.11.JNS091272. PubMed PMID: 20001590.
24. Martiniova L, Lu J, Chiang J, Bernardo M, Lonser R, Zhuang Z, Pacak K. Pharmacologic modulation of serine/threonine phosphorylation highly sensitizes PHEO in a MPC cell and mouse model to conventional chemotherapy. PLoS One. 2011; 6(2):e14678. Epub 2011/02/23. doi: 10.1371/journal.pone.0014678. PubMed PMID: 21339823; PMCID: PMC3038858.
25. Rosenberg J E, Hoffman-Censits J, Powles T, van der Heijden M S, Balar A V, Necchi A, Dawson N, O'Donnell P H, Balmanoukian A, Loriot Y, Srinivas S, Retz M M, Grivas P, Joseph R W, Galsky M D, Fleming M T, Petrylak D P, Perez-Gracia J L, Burris H A, Castellano D, Canil C, Bellmunt J, Bajorin D, Nickles D, Bourgon R, Frampton G M, Cui N, Mariathasan S, Abidoye O, Fine G D, Dreicer R. Atezolizumab in patients with locally advanced and metastatic urothelial carcinoma who have progressed following treatment with platinum-based chemotherapy: a single-arm, multicentre, phase 2 trial. Lancet. 2016; 387(10031):1909-20. Epub 2016/03/10. doi: 10.1016/S0140-6736(16)00561-4. PubMed PMID: 26952546; PMCID: PMC5480242.
26. Balar A V, Galsky M D, Rosenberg J E, Powles T, Petrylak D P, Bellmunt J, Loriot Y, Necchi A, Hoffman-Censits J, Perez-Gracia J L, Dawson N A, van der Heijden M S, Dreicer R, Srinivas S, Retz M M, Joseph R W, Drakaki A, Vaishampayan U N, Sridhar S S, Quinn D I, Duran I, Shaffer D R, Eigl B J, Grivas P D, Yu E Y, Li S, Kadel E E, 3rd, Boyd Z, Bourgon R, Hegde P S, Mariathasan S, Thastrom A, Abidoye O O, Fine G D, Bajorin D F, Group I M S. Atezolizumab as first-line treatment in cisplatin-ineligible patients with locally advanced and metastatic urothelial carcinoma: a single-arm, multicentre, phase 2 trial. Lancet. 2017; 389(10064):67-76. Epub 2016/12/13. doi: 10.1016/S0140-6736(16)32455-2. PubMed PMID: 27939400; PMCID: PMC5568632.
27. Fehrenbacher L, Spira A, Ballinger M, Kowanetz M, Vansteenkiste J, Mazieres J, Park K, Smith D, Artal-Cortes A, Lewanski C, Braiteh F, Waterkamp D, He P, Zou W, Chen D S, Yi J, Sandler A, Rittmeyer A, Group P S. Atezolizumab versus docetaxel for patients with previously treated non-small-cell lung cancer (POPLAR): a multicentre, open-label, phase 2 randomised controlled trial. Lancet. 2016; 387(10030):1837-46. Epub 2016/03/14. doi: 10.1016/S0140-6736(16)00587-0. PubMed PMID: 26970723.
28. Schmid P, Adams S, Rugo H S, Schneeweiss A, Barrios C H, Iwata H, Dieras V, Hegg R, Im S A, Shaw Wright G, Henschel V, Molinero L, Chui S Y, Funke R, Husain A, Winer E P, Loi S, Emens L A, Investigators IMT. Atezolizumab and Nab-Paclitaxel in Advanced Triple-Negative Breast Cancer. N Engl J Med. 2018; 379(22):2108-21. Epub 2018/10/23. doi: 10.1056/NEJMoa1809615. PubMed PMID: 30345906.

1. A method of treating a subject suffering from small cell lung cancer (SCLC), comprising administering to the subject an effective amount of
(a) a compound having the structure 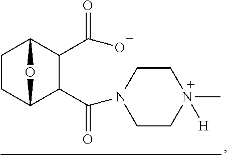
or a pharmaceutically acceptable salt or ester thereof, (b) atezolizumab, and (c) etoposide or carboplatin.

2. The method of claim 1, comprising administering to the subject etoposide and carboplatin.

3. The method of claim 1, wherein the atezolizumab and etoposide, or the atezolizumab and carboplatin, are administered simultaneously, separately or sequentially.

4. The method of claim 2, wherein the atezolizumab, etoposide and carboplatin are administered simultaneously, separately or sequentially.

5. (canceled)

6. The method of claim 1, wherein the compound or pharmaceutically acceptable salt or ester thereof is administered at a dose of about 0.83 mg/m² per day.

7. The method of claim 1, wherein the compound or pharmaceutically acceptable salt or ester thereof is administered at a dose of about 1.25 mg/m² per day.

8. The method of claim 1, wherein the compound or pharmaceutically acceptable salt or ester thereof is administered at a dose of about 1.75 mg/m² per day.

9. The method of claim 1, wherein the compound or pharmaceutically acceptable salt or ester thereof is administered at a dose of about 2.33 mg/m² per day.

10. The method of claim 1, wherein the compound or pharmaceutically acceptable salt or ester thereof is administered at a dose of about 3.10 mg/m² per day.

11. The method of claim 1, wherein the compound or pharmaceutically acceptable salt or ester thereof is administered on days 1 and 3 of a 21 day cycle.

12. The method of claim 1, wherein the compound or pharmaceutically acceptable salt or ester thereof is administered intravenously.

13. The method of claim 1, comprising administering carboplatin.

14. (canceled)

15. The method of claim 13, wherein the carboplatin is administered at a dose that achieves about AUC 5.

16. The method of claim 13, wherein the carboplatin is administered at a dose of up to about 750 mg/day.

17. The method of claim 13, wherein the carboplatin is administered on day 1 of a 21 day cycle.

18. The method of claim 17, wherein the carboplatin is administered on day 1 of a 21 day cycle for at least 4 cycles.

19. The method of claim 13, wherein the carboplatin is administered intravenously.

20. The method of claim 19, wherein the carboplatin is administered intravenously over 30-60 minutes.

21. (canceled)

22. The method of claim 1, where the atezolizumab is administered at a dose of about 1200 mg/day.

23. The method of claim 1, wherein the atezolizumab is administered on day 1 of a 21 day cycle.

24. The method of claim 23, wherein the atezolizumab is administered on day 1 of a 21 day cycle for at least 4 cycles.

25. The method of claim 1, wherein the atezolizumab is administered intravenously.

26. The method of claim 25, wherein the atezolizumab is administered intravenously over 30-60 minutes.

27. The method of claim 1, comprising administering etoposide.

28. The method of claim 27, where the etoposide is administered at a dose of about 100 mg/m² per day.

29. The method of claim 28, wherein the etoposide is administered on days 1, 2, and 3 of a 21 day cycle.

30. The method of claim 29, wherein the etoposide is administered on days 1, 2, and 3 of a 21 day cycle for at least 4 cycles.

31. The method of claim 27, wherein the etoposide is administered intravenously.

32. The method of claim 31, wherein the etoposide is administered intravenously over 60 minutes.

33. (canceled)

34. The method of claim 2, comprising administering in an order of administration the compound or pharmaceutically acceptable salt or ester thereof, followed by atezolizumab, followed by carboplatin, followed by etoposide.

35. (canceled)

36. The method of claim 1, wherein the small cell lung cancer is extensive-stage disease small cell lung cancer (ED-SCLC).

37. The method of claim 1, wherein the subject has had no prior systemic chemotherapy, immunotherapy, biological, hormonal, or investigational therapy for SCLC.

38. The method of claim 1, where the subject has not been diagnosed with non-small cell lung cancer (NSCLC) or mixed NSCLC and SCLC.
